(19) United States
(12) Patent Application Publication
(10) Pub. No.: US 2026/0258465 A1
(43) Pub. Date: Sep. 3, 2026

(54) NUCLEIC ACID-GUIDED DNA SYNTHESIS

(71) Applicant: The Trustees of Columbia University in the City of New York, New York, NY (US)

(72) Inventors: Samuel Henry Sternberg, New York, NY (US); Stephen Tang, New York, NY (US); Dennis James Zhang, New York, NY (US); Tanner Wiegand, New York, NY (US); Jerrin Thomas George, New York, NY (US); George Davis Lampe, New York, NY (US); Shannon Kang, New York, NY (US)

(21) Appl. No.: 19/672,231

(22) Filed: May 8, 2026

Related U.S. Application Data
(63) Continuation of application No. PCT/US2024/055186, filed on Nov. 8, 2024.
(60) Provisional application No. 63/597,886, filed on Nov. 10, 2023.
(60) Provisional application No. 63/640,645, filed on Apr. 30, 2024.
(60) Provisional application No. 63/657,551, filed on Jun. 7, 2024.
(60) Provisional application No. 63/667,567, filed on Jul. 3, 2024.

(51) Int. Cl. C12P 19/34; C12N 9/12

(52) U.S. Cl. CPC C12P 19/34; C12N 9/1276; C12Y 207/07049

(57) ABSTRACT

The present disclosure provides systems, compositions, methods, and kits, for guided DNA synthesis and genome engineering. Particularly, the present disclosure provides systems, compositions, methods, and kits comprising a defense-associated reverse transcriptase (DRT), or a nucleic acid encoding thereof, and an engineered target nucleic acid comprising one or more sequence of interest, or a nucleic acid encoding thereof.

### CROSS-REFERENCE TO RELATED APPLICATIONS

This application is a continuation of PCT International Application No. PCT/US2024/055186, filed Nov. 8, 2024, which claims the benefit of U.S. Provisional Application Nos. 63/597,886, filed Nov. 10, 2023, 63/640,645, filed Apr. 30, 2024, 63/657,551, filed Jun. 7, 2024, and 63/667,567, filed Jul. 3, 2024, the contents of which are herein incorporated by reference in their entirety.

### STATEMENT REGARDING FEDERALLY SPONSORED RESEARCH OR DEVELOPMENT

This invention was made with government support under GM145440 and AI183830 awarded by the National Institutes of Health. The government has certain rights in the invention.

### FIELD

The present disclosure provides systems, compositions, methods, and kits, for nucleic acid guided DNA synthesis and genome engineering.

### SEQUENCE LISTING STATEMENT

The content of the electronic sequence listing titled COLUM_42584_601_SequenceListing.xml (Size: 4,288,549 bytes; and Date of Creation: Nov. 7, 2024) is herein incorporated by reference in its entirety.

### BACKGROUND

More than 6,000 human diseases are known to stem from the mutation of a single gene. Potential strategies for curing monogenic diseases include inactivating the pathogenic gene variant, activating a functional paralogous gene, or reverting the disease-causing mutation itself back to the health sequence. With recent advances in genome engineering technologies, the implementation of such strategies has rapidly accelerated, and clinical trials for sickle cell disease, β-thalassemia, and transthyretin amyloidosis have established the feasibility of clinical gene editing.

Remarkably, discoveries of new biotechnological tools over the past half-century have repeatedly centered around restriction-modification (RM) and CRISPR-Cas revolutionizing the fields of genetic engineering and genome editing. RM and CRISPR-Cas systems share an ability to recognize and cleave nucleic acid sequences with exquisite accuracy, and whereas RM enzymes have fixed sequence specificities, Cas enzymes are easily programmed by a guide RNA (gRNA) to cleave virtually any target sequence. Repair of the resulting DNA double-strand break (DSB) via homology-directed repair (HDR) can be coopted to engineer specific genomic edits, but this process is inefficient, difficult to control, and largely restricted to mitotic cells. Furthermore, DSBs provoke the DNA damage response, cause chromosomal translocations, and can lead to large on-target deletions.

Recent efforts to systematically discover new bacterial immune mechanisms have identified numerous enzymes with previously unknown defense functions, notably including two classes of reverse transcriptase (RT) enzymes: retrons and defense-associated RTs (DRTs). While retrons have been extensively characterized since their discovery four decades ago, the specific molecular functions of DRTs are presently unknown. Retrons, which generate high copy numbers of single-stranded DNA (ssDNA) donors, have been employed to improve the efficiency of Cas9 and HDR-mediated editing. Prime editing, which utilizes an engineered viral RT tethered to a Cas9 nickase to directly synthesize new DNA at a specified target site, has similar efficiency to HDR but generates fewer unwanted byproducts. And yet despite these advances, the genome engineering toolkit remains incomplete due to a lack of approaches that avoid undesirable byproducts while still achieving high efficiency. Thus, systems, components, and methods for efficient and precise genome engineering are still needed.

### SUMMARY

Provided herein are systems, kits, compositions, and methods for guided DNA synthesis.

In some embodiments, the systems, kits, compositions, and methods comprise a defense-associated reverse transcriptase (DRT), or a nucleic acid encoding thereof; and one or more engineered target nucleic acids configured for recognition by the DRT and comprising one or more sequences of interest, or a nucleic acid encoding thereof.

In some embodiments, the DRT comprises an amino acid sequence having at least 70% identity with any of SEQ ID NOs: 1-824. In some embodiments, the DRT comprises an amino acid sequence of any of SEQ ID NOs: 1-824.

In some embodiments, the one or more engineered target nucleic acids comprise: a template region comprising the one or more sequences of interest; a 3′ region configured to recruit the DRT to the engineered target nucleic acid; and a 5′ region. In some embodiments, the template region abuts a short basal stem. In some embodiments, the 5′ end of the template sequence is homologous to the sequence adjacent to the 3′ of the template sequence in the short basal stem. In some embodiments, the 3′ region comprises one or more stem-loops. In some embodiments, the 5′ region comprises a single stem-loop.

In some embodiments, the system further comprises one or more genomic engineering reagents selected from: single-stranded annealing proteins (SSAP), guide RNAs, DNA endonucleases, ribonucleases, transcriptional activators, transcriptional repressors, histone-modifying proteins, integrases, recombinases, DNA polymerases, and combinations thereof.

Also provided herein are methods for guided DNA synthesis.

In some embodiments, the methods comprise contacting a defense-associated reverse transcriptase (DRT) with one or more engineered target nucleic acids configured for recognition by the DRT and comprising one or more sequences of interest.

In some embodiments, the methods produce a concatemeric DNA product.

In some embodiments, the DNA product comprises at least one sequence complementary to the one or more sequences of interest.

In some embodiments, the one or more engineered target nucleic acids comprise: a template region comprising the one or more sequences of interest; a 3′ region configured to recruit the DRT to the engineered target nucleic acid; and a 5′ region. In some embodiments, the template region abuts a short basal stem. In some embodiments, the 5′ end of the template sequence is homologous to the sequence adjacent to the 3′ of the template sequence in the short basal stem. In some embodiments, the 3′ region comprises one or more stem-loops. In some embodiments, the 5′ region comprises a single stem-loop.

In some embodiments, the DRT comprises an amino acid sequence having at least 70% identity with any of SEQ ID NOs: 1-824. In some embodiments, the DRT comprises an amino acid sequence of any of SEQ ID NOs: 1-824.

In some embodiments, the contacting is in a cell and the method comprised introducing into the cell the DRT and the engineered nucleic acid, or one or more nucleic acids encoding thereof. In some embodiments, the cell is in vivo. In some embodiments, the cell is in vitro. In some embodiments, the cell is a prokaryotic cell. In some embodiments, the cell is a eukaryotic cell. In some embodiments, the cell is in a subject.

Further provided herein are methods for identifying substrates and products of a target defense-associated reverse transcriptase (DRT).

In some embodiments, the methods comprise immunoprecipitating the target DRT from a first population of a plurality of cells comprising one or more putative noncoding RNAs (ncRNA) and the target DRT; isolating co-immunoprecipitated nucleic acids; and sequencing the co-immunoprecipitated nucleic acids. In some embodiments, the methods further comprise introducing into the plurality of cells one or more noncoding putative ncRNAs and the target DRT, or one or more nucleic acids encoding thereof.

In some embodiments, the DRT comprises an N-terminal or C-terminal tag for immunoprecipitation.

In some embodiments, the co-immunoprecipitated nucleic acids comprise DNA and/or RNA.

Other aspects and embodiments of the disclosure will be apparent in light of the following detailed description.

### BRIEF DESCRIPTION OF DRAWINGS

FIGS. 1A-1E shows systematic discovery of DRT2 reverse transcription substrates and products in vivo. FIG. 1A is a schematic of RNA immunoprecipitation (RIP) and cDNA immunoprecipitation (cDIP) sequencing approaches to identify nucleic acid substrates of FLAG-tagged reverse transcriptase (RT) from KpnDRT2. The plasmid-encoded immune system is schematized top left. FIG. 1B is MA plots showing the RT-mediated enrichment of RNA (top) and DNA (bottom) loci from RIP-seq and cDIP-seq experiments, relative to input controls. Each dot represents a transcript, and red dots denote transcripts with >20-fold enrichment and false discovery rate (FDR)<0.05. FIG. 1C is dRNA-seq, Term-seq, RIP-seq, and cDIP-seq coverage tracks, from top to bottom, for either WT RT or a catalytically inactive RT mutant (YCAA). dRNA-seq and Term-seq enrich RNA 5′ and 3′ ends, respectively, whereas RIP-seq and cDIP-seq identify RT-associated RNA and DNA ligands. Red and pink denote top and bottom strands, respectively, and the DRT2 locus is shown at bottom; coordinates are numbered from the beginning of the K. pneumoniae-derived sequence on the expression plasmid. Data are normalized for sequencing depth and plotted as counts per million reads (CPM). FIG. 1D is predicted secondary structure of the KpnDRT2 ncRNA (SEQ ID NO: 827). The cDNA template region is colored in pink, and the gray dotted line denotes the direction of reverse transcription. FIG. 1E is coverage over the DRT2 ncRNA locus from total DNA sequencing of cells +/−T5 phage infection (left), and bar graph of cDNA counts for the same samples alongside the YCAA mutant (right). Red and pink denote top and bottom strands, respectively; data are mean±s.d. (n=3).

FIGS. 2A-2G is rolling-circle reverse transcription that generates a concatenated cDNA product. FIG. 2A is a schematic of DRT2 ncRNA secondary structure, with stem-loops (SL) numbered 1-8 and selected perturbations highlighted in red. SL1^{MUT}, SL5^{MUT}, and SL6^{MUT} correspond to ncRNA mutants in which the SL bases were scrambled, resulting in the elimination of sequence motifs and secondary structure. SL2^{MUT} abolishes base pairing within the SL2 stem. Sequences of all mutants are presented in Table 3. FIG. 2B is a plaque assay showing loss of phage defense activity for all SL mutants from A (left), and bar graph quantifying the reduction in efficiency of plating (EOP, right); data are mean±s.d. (n=3). FIG. 2C is RIP-seq and cDIP-seq coverage tracks for the indicated SL mutants alongside input controls, revealing a range of defects in either RNA binding, cDNA synthesis/binding, or both. FIG. 2D Top: Schematic of terminal portions of cDIP-seq reads (light gray) failing to align to the cDNA reference, resulting in ‘soft clipping’ and exclusion from coverage plots. A donut plot reporting the proportion of cDNA-mapping reads with the indicated lengths of 3′-clipped sequences is shown at left for DRT2 WT cDIP-seq. Bottom: Mapping of 3′-soft-clipped sequences from cDIP-seq experiments back to the DRT2 locus, demonstrating that they derive from the cDNA 5′ end. SL2^{MUT} exhibits an aberrant pattern relative to WT. FIG. 2E is a schematic of sequencing reads that map across two concatenated cDNA repeats (top), and bar graph quantifying the abundance of junction-spanning reads from sequencing of total DNA in the indicated conditions (bottom). Red and pink denote top and bottom strands, respectively; data are mean±s.d. (n=3). FIG. 2F is a schematic of long-read Nanopore sequencing workflow with DNA from phage-infected cells (top), and histogram of cDNA repeat length distribution for WT KpnDRT2 from Nanopore sequencing (bottom). FIG. 2G is an inferred mechanism of rolling-circle reverse transcription (RCRT) mediated by sequence and structural features of SL2. After synthesis of 5′-TGT-3′ templated by ACA-1 at the end of one cDNA repeat (top), the nascent DNA strand dissociates from its template and reanneals with the complementary ACA-2 following SL2 melting (middle). Template jumping initiates a subsequent round of reverse transcription, with concatenation of one cDNA repeat to the next and incorporation of one additional base at the repeat junction, ultimately leading to long rolling-circle cDNA products (bottom).

FIGS. 3A-3G shows the concatenated cDNA product contains a never-ending ORF (neo). FIG. 3A is a bar graph quantifying RNA-seq reads that map across two concatenated cDNA repeats, for the indicated conditions. Red and pink denote top and bottom strands, respectively; data are mean±s.d. (n=3). FIG. 3B is a model showing the consecutive production of ncRNA (transcription), concatenated double-stranded cDNA (reverse transcription and second-strand synthesis), and concatenated RNA (transcription), all encoded by the DRT2 locus. Dashed lines indicate repeat-repeat junctions resulting from rolling-circle reverse transcription, and the inset (top left) shows the predicted promoter formed across the junction. FIG. 3C is a bar graph quantifying relative concatenated RNA abundance in a phage infection time course experiment using RT-qPCR with repeat junction primers (top), and Northern blot of concatenated RNA using a junction-spanning probe (bottom). RT-qPCR data are normalized to WT uninfected cells (t=0); data are mean±s.d. (n=3). FIG. 3D shows the putative open reading frame (ORF) (SEQ ID NO: 3408) encoded by the concatenated RNA (SEQ ID NO: 3409). The start of cDNA synthesis and putative start of translation are indicated (pink and blue arrows, respectively), and the repeat-repeat junction is denoted with a dashed line. FIG. 3E is a schematic of the cDNA template region (pink), with the putative start codon and experimentally tested mutations indicated; (SEQ ID NO: 845). FIG. 3F is a plaque assay showing that phage defense activity is eliminated with a single-bp substitution that introduces an in-frame stop codon, but is only modestly affected by synonymous or missense mutations. EV, empty vector. FIG. 3G is a bar graph quantifying phage defense activity for insertions within SL3, SL4, or SL5, of the indicated length. Reduction in EOP is calculated relative to an EV control; data are mean±s.d. (n=3). The only mutants that retain phage defense activity have insertion lengths of a multiple of 3 bp.

FIGS. 4A-4H show Neo proteins induce programmed cellular dormancy. FIG. 4A is a schematic of experimental approach to detect Neo in phage-infected cells by liquid chromatography with tandem mass spectrometry (LC-MS/MS). FIG. 4B is a bar graph quantifying Neo protein quantity from cells tested in the indicated conditions. Data are mean±s.d. (n=3). FIG. 4C shows the abundance of RT and Neo proteins relative to the E. coli proteome in phage-infected cells expressing WT DRT2. FIG. 4D shows differential protein abundance in T5-infected cells expressing DRT2 WT or YCAA. Phage proteins are colored in brown, and ArfA and RMF are colored in red and labeled. All other differentially abundant proteins (fold change >2 and FDR<0.05) are colored in dark blue. FIG. 4E is a schematic of alternative ribosome rescue pathway mediated by ArfA, which would release Neo proteins from ribosomes stalled on non-stop neo mRNAs without targeting them for degradation (right), unlike the tmRNA pathway (left). FIG. 4F shows the growth curves of strains transformed with empty vector (EV) or the WT DRT2 system, +/−T5 phage at the indicated multiplicity of infection (MOI). Shaded regions indicate the standard deviation across independent biological replicates (n=3). FIG. 4G is a schematic of cloning and inducible expression strategy to monitor the physiological effects of Neo polypeptides of variable repeat length. FIG. 4H is growth curves of strains transformed with WT or scrambled Neo sequences of the indicated repeat lengths, alongside an empty vector (EV) control. The dashed line indicates the point of induction with arabinose and theophylline. Shaded regions indicate the standard deviation across independent biological replicates (n=3).

FIGS. 5A-5H show concatenated neo genes and programmed dormancy are a broadly conserved phage defense mechanism. FIG. 5A is a schematic for the automated detection of putative Neo proteins in homologous DRT2 operons (SEQ ID NO: 3410-3415). FIG. 5B is a phylogenetic tree of KpnDRT2 homologs, with outer rings showing the widespread presence of RT-associated ncRNAs and putative Neo proteins. Homologs selected for experimental testing are indicated with pink circles. FIG. 5C shows the multiple sequence alignment (MSA) and secondary structure prediction of Neo proteins identified (SEQ ID NOs: 3416-3421) in FIG. 5B. A single Neo repeat is shown for all homologs; shading indicates amino acid conservation. FIG. 5D is an AlphaFold prediction of a 3-repeat Neo polypeptide, showing the sites of proline mutagenesis tested in FIG. 5E. Prolines were inserted C-terminal to the indicated residues within each of 3 concatenated repeats. FIG. 5E is growth curves of strains transformed with 3-repeat Neo constructs containing the indicated proline insertions, alongside an empty vector (EV) control. The dashed line indicates the point of induction with arabinose and theophylline. Shaded regions indicate the standard deviation across independent biological replicates (n=3). FIG. 5F is a heat map showing the distribution of neo cDNA repeat lengths in cells expressing the indicated DRT2 homologs. Data are plotted as log₁₀(CPM) from Nanopore sequencing of total DNA. FIG. 5G is a heat map showing the growth rates of cells expressing Neo homologs with the indicated repeat lengths. Growth rates are normalized to an EV control and represent the mean of independent biological replicates (n=3). Empty cells with X indicate Neo expression constructs that could not be successfully cloned, presumably due to toxicity. FIG. 5H is an exemplary model for antiphage defense mechanism of DRT2 systems. RT enzymes bind the scaffold portion of associated ncRNAs and constitutively produce concatenated cDNA products via rolling-circle reverse transcription (RCRT). Phage infection triggers second-strand synthesis, yielding a dsDNA molecule that is transcribed into never-ending ORF (neo) mRNAs. Neo translation exploits a ribosome rescue pathway to produce Neo proteins that potently arrest cell growth, protecting the larger bacterial population from the spread of phage.

FIGS. 6A-6E is an overview and validation of cDNA immunoprecipitation and sequencing (cDIP-seq) approach using Retron-Ecol. FIG. 6A is a summary of operonic configurations for DRT systems with experimentally validated phage defense activity. Protein domains and associated ncRNAs are indicated. FIG. 6B is a detailed overview of combined RIP-seq and cDIP-seq workflow. FIG. 6C is a plaque assay showing that FLAG-tagged RT proteins in Retron-Ecol (formerly Ec86) and KpnDRT2 systems retain WT defense activity. FIG. 6D is a schematic of the Retron-Ecol operon (left), retron-encoded ncRNA and nascent reverse transcript (middle), and mature multi-copy single-stranded DNA (msDNA) resulting from reverse transcription and RNase H processing steps (right). FIG. 6E is RIP-seq and cDIP-seq coverage tracks for Retron-Ecol, plotted alongside corresponding input controls over the plasmid-encoded Retron-Ecol locus. The drop-off in RIP coverage is consistent with processing of the msRNA by RNase H. The magnified insets highlight the accuracy and single-nucleotide precision of cDIP-seq in identifying the msDNA (SEQ ID NOs: 3422-3423).

FIGS. 7A-7E show additional analyses of DRT2 RIP-seq and cDIP-seq data. FIG. 7A is MA plots showing catalytically inactive RT (YCAA)-mediated enrichment of RNA (left) and DNA (right) loci from RIP-seq and cDIP-seq experiments, relative to input controls. Each dot represents a transcript, and red dots (RIP-seq) or pink dots (cDIP-seq) denote transcripts with >20-fold enrichment and false discovery rate (FDR)<0.05. The cDIP-seq enrichment of lacZ and racR with an RT-inactive mutant indicates that they do not represent true cDNA synthesis products. FIG. 7B is MA plots as in FIG. 7A for RIP-seq and cDIP-seq of WT DRT2 from T5 phage-infected cells. Transcripts from the ncRNA locus are similarly enriched as in RIP-seq and cDIP-seq experiments from uninfected cells (FIG. 1i), but enrichment of diverse tRNAs in the RIP-seq dataset was also observed. FIG. 7C is a histogram of mapping coordinates for 5′ and 3′ ends of cDIP-seq fragments visualized over the DRT2 ncRNA locus (bottom). Coordinates are numbered from the beginning of the K. pneumoniae-derived sequence on the DRT2 expression plasmid; the precise coordinates are indicated next to their corresponding peaks. FIG. 7D is a covariance model for ncRNA sequences from KpnDRT2 and related loci (n=303). PK denotes a predicted pseudoknot interaction between the indicated regions. FIG. 7E is RIP-seq and cDIP-seq coverage tracks for either WT RT or a catalytically inactive RT mutant (YCAA) from T5 phage-infected cells. Red and pink denote top and bottom strands, respectively, and the DRT2 locus is shown at bottom. Data are normalized for sequencing depth and plotted as counts per million reads (CPM); coordinates are numbered as in FIG. 7C.

FIGS. 8A-8F show molecular dissection of phage and ncRNA requirements during rolling-circle reverse transcription (RCRT). FIG. 8A is a schematic of the DRT2 ncRNA secondary structure, indicating the region surrounding the reverse transcription start site (red line) that was mutated for RIP-seq, cDIP-seq, and phage defense experiments. FIG. 8B is RIP-seq and cDIP-seq coverage tracks for the scrambled cDNA start region mutant (cDNA start^{MUT}) alongside input controls, showing RNA binding and cDNA synthesis activities comparable to WT (FIG. 1C). FIG. 8C is a plaque assay demonstrating loss of phage defense activity with the cDNA start region mutant. FIG. 8D is a stacked bar graph quantifying cDIP-seq reads mapping across the cDNA repeat-repeat junction as a proportion of total reads mapping to the DRT2 ncRNA locus, for the WT system and indicated ncRNA SL mutants in uninfected cells. The data demonstrate that programmed template jumping requires an intact SL2 alongside conserved ncRNA features at the 5′ end (SL1) and within the scaffold region (SL6). FIG. 8E is a bar graph quantifying the abundance of junction-spanning reads from cDIP-seq experiments with the indicated conditions. Red and pink denote top and bottom strands, respectively; data are mean±s.d. (n=3). Based on the difference between these results and those obtained from total DNA sequencing (FIG. 2E), the RT likely releases double-stranded cDNA after second-strand synthesis, leading to a lack of enrichment for the top strand in these experiments. FIG. 8F is a plaque assay showing loss of phage defense activity with an additional SL2 mutant (SL2^{MUT}-2), as well as with mutations to either ACA-1 or ACA-2 (see FIG. 2G). Mutated nucleotides are bolded in bright red, template ncRNA nucleotides are in maroon, and cDNA nucleotides are in pink.

FIGS. 9A-9E show additional genetic evidence that the DRT2 concatenated cDNA encodes a functional open reading frame (ORF). FIG. 9A is in silico translation of the cDNA repeat sequence in all three possible reading frames, demonstrating that Frame 1 lacks stop codons. Frame 1—SEQ ID NOs: 3424, amino acid, and 3428, DNA. Frame 2—SEQ ID NOs: 3410, 3411, and 3425-3426, amino acid, and 3428, DNA. Frame 3—SEQ ID NOs: 3412 and 3427, amino acid, and 3428, DNA. FIG. 9B shows the prediction of translation initiation efficiency for an mRNA derived from one cDNA repeat, based on an in silico ribosome binding site (RBS) calculation tool. Predicted values are shown in arbitrary units (au) for each potential AUG start codon, as well as the non-canonical start codons GUG and UUG, found within the mRNA. FIG. 9C is a schematic of the cDNA template region (pink), with experimentally tested mutations indicated (SEQ ID NO: 3429). Single-bp substitutions were designed to either disrupt the putative start codon or introduce a missense or stop codon after one near-full-length neo repeat (39/40 amino acids). FIG. 9D is a plaque assay demonstrating that mutation of the putative AUG start codon to GUG, a common non-canonical start codon in E. coli, is partially tolerated, whereas mutations to UUG and CUG result in a complete loss of defense. FIG. 9E is a plaque assay demonstrating that a single-bp substitution at G112 is partially tolerated when resulting in a missense codon, but abolishes defense activity when resulting in a stop codon.

FIGS. 10A-10F show detection and recombinant expression of neo. FIG. 10A is a cleavage map for Neo digestion using a custom 3-enzyme protease cocktail prior to liquid chromatography with tandem mass spectrometry (LC-MS/MS) analysis. A full Neo repeat is shown, together with the portion of the downstream repeat leading up to the next cleavage site. Cleavage sites are indicated with triangles, colored by enzyme; the ideal peptide fragment size for MS is 9-15 amino acids. SEQ ID NO: 3430. FIG. 10B shows T5 phage titer measurements at the indicated time points after a high-MOI infection of cells expressing DRT2 (WT or YCAA). Data are shown as mean±s.d. (n=3). FIG. 10C, left, is growth curves of strains transformed with empty vector (EV) or the WT DRT2 system, +/−T5 phage infection at an MOI of 5. FIG. 10C, right, shows relative fluorescence unit (RFU) measurements after addition of resazurin to the same cultures after 3 hours of growth. The shaded regions indicate the standard deviation across independent biological replicates (n=6). FIG. 10D shows Sanger sequencing traces showing the presence of frameshift mutations in neo when attempting to isolate clones of a 3-repeat neo expression vector. SEQ ID NO: 3431, amino acid, and SEQ ID NOs: 3432-3434, DNA. FIG. 10E shows colony PCR demonstrating unsuccessful cloning of 3-repeat WT neo into a standard expression vector, as compared to successful cloning of 3-repeat scrambled neo mutant. For each experiment, 8 clones were randomly selected and subjected to PCR, which is expected to generate a ~900-bp band with the insert. FIG. 10F shows colony forming unit (CFU) measurements for cultures from FIG. 4H plated on repressor (2% glucose) or inducer (0.5% arabinose and 0.5 mM theophylline) after the final OD₆₀₀ measurement time point.

FIGS. 11A-11D show widespread presence of neo across diverse clades of DRT2 systems. FIG. 11A is a phylogenetic tree of DRT2-encoded RT homologs (n=2,116). The rings depict ncRNA sequences identified via CM searches (inner) and the presence of bioinformatically identified neo genes (outer). The subtree shown in FIG. 5B comprises the sequences highlighted in light blue. FIG. 11B, top: Conservation of ACA-1 and ACA-2 motifs that mediate programmed template jumping (n=257 loci); (SEQ ID NO: 827). FIG. 11B, bottom: Conservation of −35 and −10 promoter elements flanking the neo repeat junction (n=203 loci). FIG. 11C is exemplary covariance models for the DRT2 ncRNA derived from additional clades in A; the locations of each CM within the phylogenetic tree are indicated. FIG. 11D is additional in silico predictions of the 3D structure of a 3-repeat Neo polypeptide. Kpn Neo was used as a single-sequence input for ESMFold, while an MSA of the homologs shown in FIG. 5C was used as input for trRosetta.

FIGS. 12A and 12B show diverse neo homologs induce repeat length-dependent growth arrest. FIG. 12A is sequence identity matrices of the RT, ncRNA, cDNA repeat, and Neo polypeptide sequences for experimentally tested DRT systems. FIG. 12B is growth curves of strains transformed with neo homologs of the indicated repeat lengths, alongside an empty vector (EV) control (related to FIG. 5G). Expression was induced with arabinose and theophylline as indicated. Shaded regions indicate the standard deviation across independent biological replicates (n=3). Note that cloning of neo from Y. ruckeri with 3 or 4 repeats was unsuccessful, presumably due to toxicity from even minimal leaky expression of the Neo protein.

FIG. 13 shows phage defense activity of selected DRT2 homologs. Plaque assays evaluating defense activity in E. coli MG1655 cells transformed with plasmids encoding KpnDRT2 or EcoDRT2, compared to cells transformed with an empty vector (EV) control. Lawns of bacteria were spotted with 10-fold serial phage dilutions, decreasing in concentration from left to right. The relative absence of T5 plaques in the presence of KpnDRT2 or EcoDRT2 compared to the EV control indicates defense against T5. Therefore, based on the mechanism described herein for KpnDRT2, this is indicative of active rolling-circle reverse transcription for both KpnDRT2 and EcoDRT2.

FIGS. 14A-14D show in vitro reconstitution of DRT2 reverse transcription activity. FIG. 14A shows the binding of ncRNA to DRT2 visualized by electrophoretic mobility shift assay. ncRNA was titrated with DRT2 and the bound complex was resolved by native PAGE. FIG. 14B shows in vitro reverse transcription reaction revealing near complete consumption of the ncRNA and formation of cDNA products as bands of higher and lower mobility. ncRNA and DRT2 was incubated with dNTPs for various time points and the product was visualized on denaturing PAGE.

FIG. 14C shows in vitro reverse transcription reaction quenched after 15 min incubation (lane 2) and separately treated with RNase H (lane 3), RNase A (lane 4), dsDNase (lane 5), DNase I (lane 6) or proteinase K (lane 7). The digested products were visualized by denaturing PAGE. Control in vitro reverse transcription reaction quenched immediately after adding dNTPs shows the starting ncRNA as the major band (lane 1). Lane 6 reveals covalently linked ncRNA and cDNA product. FIG. 14C shows PCR with products of in vitro reverse transcription reaction revealing bands corresponding to first (cDNA-1), second (cDNA-2) and third (cDNA-3) round of rolling circle reverse transcription.

FIG. 15 shows a comparison of Nanopore read length distribution for concatemeric cDNA (ccDNA) reads compared to total reads. The median length for ccDNA reads is 479 nt, and the median length for total reads is 833 nt. Considering the similarity between the distributions, the observed ccDNA lengths could reflect technical limitations of Nanopore sequencing rather than the true length distribution in cells, which may skew longer than observed here.

FIGS. 16A and 16B show the error rate of KpnDRT2 reverse transcriptase. FIG. 16A shows the frequency distribution for errors per read from cDIP-seq analysis of KpnDRT2. Plotted along the x-axis is the number of errors within a read with respect to the reference sequence, and along the y-axis is the frequency of reads with the given number of errors, as a proportion of the total reads. The baseline sequencing error frequency (background) is determined by analyzing input controls from cDIP-seq experiments, considering the reads for such samples that map to the plasmid encoding KpnDRT2 but not mapping to the cDNA locus itself. The reverse transcription error frequency (cDNA) is determined by analyzing KpnDRT2 cDIP-seq reads mapping to the cDNA locus. FIG. 16B shows the error rate calculation for reverse transcription by KpnDRT2. Error rate is calculated using the same reads as described in FIG. 16A, and is given as the number of errors per base. The error rate of cDNA above background, calculated as 1.64×10⁻³ errors per base, represents the error rate of the KpnDRT2 reverse transcriptase.

FIGS. 17A and 17B show the identification of putative DRT2 triggers by screening of escaper phages. FIG. 17A shows plaque assays demonstrating that T5 escaper phages (T5.e1-T5.e8) are not sensitive to the presence of the DRT2 defense system. Whereas wild-type T5 phages (T5.WT) form plaques less efficiently on DRT2-expressing cells than an empty vector (EV) control, escaper phages exhibit similar plaquing efficiencies between the two conditions. FIG. 17B shows sequencing of T5 escaper phages reveals missense mutations in genes encoding putative triggers of DRT2 immune activity. Top: Five escaper phages are mutated in the dmp gene. Middle: Two escaper phages are mutated in the D11 gene. Bottom: One escaper phage is mutated in the A1 gene. Positions of mutations in the T5 genome, and their effects on the coding sequence for each respective gene, are indicated in the schematic below each sequencing track.

FIG. 18 is KpnDRT2 construct design and the predicted ncRNA (SEQ ID NO: 827) secondary structure. The ncRNA contains a cDNA template region and scaffold region, and the RT is encoded downstream of the ncRNA (above). Stem-loops (SL), ACA motifs (ACA-1 and ACA-2), ncRNA base coordinates, and the template (red) vs. scaffold (grey) regions are annotated on the secondary structure. Coordinates of the ncRNA are labeled from 1 to 281 in SEQ ID NO: 845, starting with the 5′ nucleotide.

FIGS. 19A-19C show in vivo cDNA synthesis data for KpnDRT2 ncRNA scaffold mutants. FIG. 19A is sequencing read coverage plotted over the KpnDRT2 ncRNA locus for the indicated ncRNA variants. Coordinates are numbered with respect to the first nucleotide of the plasmid as listed in Table 4. FIG. 19B is total reverse transcription activity quantified by counting sequencing reads that map to the cDNA locus. FIG. 19C is RCRT activity quantified by counting sequencing reads that map across the cDNA repeat-repeat junction.

FIGS. 20A and 20B show in vivo cDNA synthesis data for KpnDRT2 ncRNA template jumping junction mutants. FIG. 20A shows total reverse transcription activity was quantified by counting sequencing reads that map to the cDNA locus. FIG. 20B shows RCRT activity was quantified by counting sequencing reads that map across the cDNA repeat-repeat junction. WT and scramble_SL6 substrates are shown for comparison; the WT ncRNA exhibits fully active cDNA synthesis and RCRT activity, while the scramble_SL6 variant represents inactive cDNA synthesis and RCRT. Total cDNA counts will also include plasmid-derived reads, and therefore scramble_SL6 can be taken to represent ‘background’ signal.

FIGS. 21A-21G show in vivo cDNA synthesis data for KpnDRT2 ncRNA template sequence mutants. FIG. 21A is a schematic of ncRNA (SEQ ID NO: 3435) variants in which segments of the template region were mutated to random sequences in multiples of 20-nt. A dashed box is shown around the portion of the ncRNA that was mutated for each variant. The template region is demarcated by solid vertical lines, and the coordinates of the first and last nucleotides of the template region are indicated, numbered with respect to the start of the ncRNA. FIG. 21B is sequencing read coverage plotted over the KpnDRT2 ncRNA locus for the indicated ncRNA variants. Coordinates are numbered with respect to the first nucleotide of the plasmid as listed in Table 4. FIGS. 21C and 21D show total reverse transcription activity quantified by counting sequencing reads that map to the cDNA locus. FIGS. 21E and 21F shows RCRT activity quantified by counting sequencing reads that map across the cDNA repeat-repeat junction. WT and scramble_SL6 substrates are shown for comparison; the WT ncRNA exhibits fully active cDNA synthesis and RCRT activity, while the scramble_SL6 variant represents inactive cDNA synthesis and RCRT. Note that total cDNA counts will also include plasmid-derived reads, and therefore scramble_SL6 can be taken to represent ‘background’ signal. FIG. 21G is a schematic of the KpnDRT2 ncRNA (SEQ ID NO: 827) with the regions that differ between the ‘rand100’ variant and ‘rand117’ variant indicated with a dashed line.

FIGS. 22A and 22B show in vivo cDNA synthesis data for KpnDRT2 ncRNA template structure mutants. FIG. 22A shows total reverse transcription activity quantified by counting sequencing reads that map to the cDNA locus. FIG. 22B shows RCRT activity quantified by counting sequencing reads that map across the cDNA repeat-repeat junction. WT, scramble_SL6, and rand100 substrates are shown for comparison; the WT ncRNA exhibits fully active cDNA synthesis and RCRT activity, while the scramble_SL6 variant represents inactive cDNA synthesis and RCRT. Total cDNA counts will also include plasmid-derived reads, and therefore scramble_SL6 can be taken to represent ‘background’ signal.

FIGS. 23A-23D show in vivo cDNA synthesis data for KpnDRT2 ncRNA template structure mutants. FIGS. 23A and 23B show total reverse transcription activity quantified by counting sequencing reads that map to the cDNA locus. FIGS. 23C and 23D show RCRT activity quantified by counting sequencing reads that map across the cDNA repeat-repeat junction. WT and scramble_SL6 substrates are shown for comparison; the WT ncRNA exhibits fully active cDNA synthesis and RCRT activity, while the scramble_SL6 variant represents inactive cDNA synthesis and RCRT. Total cDNA counts will also include plasmid-derived reads, and therefore scramble_SL6 can be taken to represent ‘background’ signal.

FIGS. 24A and 24B show strand-specific in vivo cDNA synthesis data for KpnDRT2 ncRNA mutants. Sequencing reads mapping to the cDNA locus (FIG. 24A) or specifically to the cDNA repeat-repeat junction (FIG. 24B) were counted based on their strandedness. First-strand cDNA reads are shown in pink, while second-strand cDNA reads are shown in maroon.

FIGS. 25A and 25B show a schematic of an exemplary strategy for reconstitution of nucleic acid-guided DNA synthesis activity of DRT2 in human cells. FIG. 25A is a workflow for expressing DRT2 system components in human cells by transfection, followed by analysis of reverse transcription activity by RIP-seq and cDIP-seq. FIG. 25B shows the native KpnDRT2 ncRNA locus at top, with the experimentally determined ncRNA and cDNA template boundaries indicated. The three human cell expression vector designs are shown below. The first design (ncRNA-1, encoded by pSL7516) places a self-cleaving ribozyme immediately downstream of the ncRNA 3′ end. The second design (ncRNA-2, encoded by pSL7517) places a poly-T terminator immediately downstream of the ncRNA 3′ end. The third design (ncRNA-3, encoded by pSL7518) includes the native KpnDRT2 sequence between the ncRNA 3′ end and the RT ORF, upstream of the poly-T terminator.

FIGS. 26A-26E show quantification of total cDNA synthesis and rolling circle reverse transcription (RCRT) by KpnRT and its associated ncRNA in HEK293T cells. FIG. 26A shows reads mapping to the plasmid-encoded KpnDRT2 cDNA locus quantified from cDIP-seq experiments, for the indicated transfection conditions (−/+RT expression vector with co-transfection of ncRNA-1, ncRNA-2, or ncRNA-3 expression vector). Input (non-immunoprecipitated) controls for each cDIP-seq experiment are shown. Data are normalized for sequencing depth and plotted as counts per million reads (CPM). FIG. 26B shows reads mapping across the repeat-repeat junction in a concatenated cDNA reference quantified for the same conditions as in FIG. 26A. Data are normalized for sequencing depth and plotted as counts per million reads (CPM). FIG. 26C shows reads mapping to the KpnDRT2 cDNA locus, with strandedness corresponding to the second strand of cDNA synthesis, quantified for the same conditions as in FIG. 26A. Data are shown as in FIG. 26A. FIG. 26D shows reads mapping across the repeat-repeat junction in a concatenated cDNA reference, with strandedness corresponding to the second strand of cDNA synthesis, quantified for the same conditions as in FIG. 26A. Data are shown as in FIG. 26B. FIG. 26E shows sequencing read coverage plotted over the plasmid-encoded KpnDRT2 ncRNA locus for RIP-seq and cDIP-seq experiments with co-transfection of RT expression vector and the indicated ncRNA expression vector. Coordinates are numbered with respect to the first nucleotide of the ncRNA expression plasmid as listed in Table 4.

FIGS. 27A and 27B show transcriptome-wide analysis of KpnRT substrate specificity in human cells. FIG. 27A is a MA plot showing fold enrichment of annotated transcripts by KpnRT RIP-seq, relative to an input control. Cells were transfected with the KpnRT expression plasmid and the ncRNA-3 expression plasmid. FIG. 27B is a MA plot showing fold enrichment of annotated transcripts by KpnDRT2 cDIP-seq relative to an input control. Cells were transfected with the KpnRT expression plasmid and the ncRNA-3 expression plasmid. For A and B, loci with fold enrichment >5 are colored in red.

FIGS. 28A-28C show in vivo cDNA synthesis data for KpnDRT2 with 3′ truncation of the ncRNA. FIG. 28A is sequencing read coverage plotted over the KpnDRT2 ncRNA locus for the indicated conditions. FIG. 29B is a graph of total cDNA synthesis activity for the indicated conditions quantified by counting sequencing reads that map to the cDNA locus. FIG. 29C is RCRT activity for the indicated conditions quantified by counting sequencing reads that map across the cDNA repeat-repeat junction. WT and YCAA samples are shown for comparison; the WT system exhibits fully active cDNA synthesis and RCRT activity, while the YCAA system (KpnDRT2 with a catalytically inactive RT) represents inactive cDNA synthesis and RCRT. Note that total cDNA counts will also include plasmid-derived reads, and therefore the YCAA condition represents background signal.

FIGS. 29A and 29B show in vivo cDNA synthesis data for KpnDRT2 ncRNA template jumping junction mutants. FIG. 29A is a graph of total cDNA synthesis activity for the indicated conditions quantified by counting sequencing reads that map to the cDNA locus. FIG. 29B is a graph of RCRT activity for the indicated conditions quantified by counting sequencing reads that map across the cDNA repeat-repeat junction. WT and YCAA samples are shown for comparison; the WT system exhibits fully active cDNA synthesis and RCRT activity, while the YCAA system (KpnDRT2 with a catalytically inactive RT) represents inactive cDNA synthesis and RCRT. Note that total cDNA counts will also include plasmid-derived reads, and therefore the YCAA condition represents background signal.

FIGS. 30A-30E show in vivo cDNA synthesis data for KpnDRT2 ncRNA template length mutants. FIG. 30A is a graph of total cDNA synthesis activity for the indicated conditions quantified by counting sequencing reads that map to the cDNA locus. FIG. 30B is a graph of RCRT activity for the indicated conditions quantified by counting sequencing reads that map across the cDNA repeat-repeat junction. FIG. 30C is a schematic of ncRNA variants (SEQ ID NO: 3435) with increasingly large deletions of the template region. A dashed box is shown around the portion of the ncRNA that was deleted for each variant. The template region is demarcated by solid vertical lines, and the coordinates of the first and last nucleotides of the template region are indicated, numbered with respect to the start of the ncRNA. FIG. 30D is a graph of total cDNA synthesis activity for the indicated deletion variants quantified by counting sequencing reads that map to the cDNA locus. FIG. 30E is a graph of RCRT activity for the indicated deletion variants quantified by counting sequencing reads that map across the cDNA repeat-repeat junction. WT and YCAA samples are shown for comparison; the WT system exhibits fully active cDNA synthesis and RCRT activity, while the YCAA system (KpnDRT2 with a catalytically inactive RT) represents inactive cDNA synthesis and RCRT. Note that total cDNA counts will also include plasmid-derived reads, and therefore the YCAA condition represents background signal.

FIGS. 31A and 31B show the assessment of KpnDRT2 template jumping versus template switching activity in vivo. FIG. 31A is a schematic of experimental design to assess cis versus trans template jumping by KpnDRT2. Cells were co-transformed with the two plasmids shown, resulting in expression of the RT and two distinct ncRNA templates. RT template jumping leads to multiple possible concatenation outcomes, with “TS_SL4_SL5” representing trans template jumping, or template switching. FIG. 31B is a graph of sequencing reads corresponding to the concatenation outcomes schematized in FIG. 31A counted for the indicated conditions. In conditions 1-4, cells were co-transformed with an empty vector (EV) or RT expression vector, along with a ncRNA expression vector encoding one or two ncRNAs. In condition 5, DNA purified from conditions 3 and 4 was mixed prior to library preparation and sequencing.

FIGS. 32A and 32B show reconstitution of nucleic acid-guided DNA synthesis activity by diverse DRT2 homologs in human cells. FIG. 32A is a graph of total cDNA synthesis activity for the indicated homologs quantified by counting cDIP-seq reads mapping to the cDNA locus. FIG. 32B is a graph of RCRT activity for the indicated homologs quantified by counting cDIP-seq reads that map across the cDNA repeat-repeat junction. Experiments were performed with or without co-transfection of the RT expression plasmid. Counts from samples without the RT expression plasmid (−RT) represent background signal.

### DETAILED DESCRIPTION

The disclosed systems, compositions, methods, and kits utilize defense-associated reverse transcriptases (DRT) for guided DNA synthesis, for example for use in genome engineering. The highly efficient rolling-circle reverse transcription (RCRT) activity of DRT2 family members represents a unique biochemical behavior that produces concatenated, repetitive cDNA molecules with precise junction sequences, expanding the diversity of products that can be generated by a single polymerase enzyme from its substrate. DRT2 represents the first biological system in which a reverse transcriptase natively performs RCRT. Intriguingly, it accomplishes this using a template that is not a closed circle, and thus differs from classic examples of rolling circle amplification associated with plasmid, phage, and viroid replication. These unique properties of DRT2-encoded enzymes offer considerable potential for biotechnology applications that leverage templated DNA production in vivo, but with the added advantage of programmed amplification.

Section headings as used in this section and the entire disclosure herein are merely for organizational purposes and are not intended to be limiting.

### Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. As used herein, comprising a certain sequence or a certain SEQ ID NO usually implies that at least one copy of said sequence is present in recited peptide or polynucleotide. However, two or more copies are also contemplated. The singular forms “a,” “and” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. For example, any nomenclature used in connection with, and techniques of cell and tissue culture, molecular biology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

As used herein, “nucleic acid” or “nucleic acid sequence” refers to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 41(14): 4503-4510 (2002)) and U.S. Pat. No. 5,034,506), locked nucleic acid (LNA; see Wahlestedt et al., Proc. Natl. Acad. Sci. U.S.A., 97: 5633-5638 (2000)), cyclohexenyl nucleic acids (see Wang, J. Am. Chem. Soc., 122: 8595-8602 (2000)), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand. The terms “nucleic acid,” “polynucleotide,” “nucleotide sequence,” and “oligonucleotide” are used interchangeably. They refer to a polymeric form of nucleotides of any length, either deoxyribonucleotides or ribonucleotides, or analogs thereof.

Nucleic acid or amino acid sequence “identity,” as described herein, can be determined by comparing a nucleic acid or amino acid sequence of interest to a reference nucleic acid or amino acid sequence. A number of mathematical algorithms for obtaining the optimal alignment and calculating identity between two or more sequences are known and incorporated into a number of available software programs. Examples of such programs include CLUSTAL-W, T-Coffee, and ALIGN (for alignment of nucleic acid and amino acid sequences), BLAST programs (e.g., BLAST 2.1, BL2SEQ, and later versions thereof) and FASTA programs (e.g., FASTA3x, FAS™, and SSEARCH) (for sequence alignment and sequence similarity searches). Sequence alignment algorithms also are disclosed in, for example, Altschul et al., J. Molecular Biol., 215(3): 403-410 (1990), Beigert et al., Proc. Natl. Acad. Sci. USA, 106(10): 3770-3775 (2009), Durbin et al., eds., Biological Sequence Analysis: Probabilistic Models of Proteins and Nucleic Acids, Cambridge University Press, Cambridge, UK (2009), Soding, Bioinformatics, 21(7): 951-960 (2005), Altschul et al., Nucleic Acids Res., 25(17): 3389-3402 (1997), and Gusfield, Algorithms on Strings, Trees and Sequences, Cambridge University Press, Cambridge UK (1997)).

The terms “non-naturally occurring,” “engineered,” and “synthetic” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

A “vector” or “expression vector” is a replicon, such as plasmid, phage, virus, or cosmid, to which another DNA segment, e.g., an “insert,” may be attached or incorporated so as to bring about the replication of the attached segment in a cell.

A cell has been “genetically modified,” “transformed,” or “transfected” by exogenous DNA, e.g., a recombinant expression vector, when such DNA has been introduced inside the cell. The presence of the exogenous DNA results in permanent or transient genetic change. The transforming DNA may or may not be integrated (covalently linked) into the genome of the cell. For example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones that comprise a population of daughter cells containing the transforming DNA. A “clone” is a population of cells derived from a single cell or common ancestor by mitosis. A “cell line” is a clone of a primary cell that is capable of stable growth in vitro for many generations.

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment of the methods and compositions provided herein, the mammal is a human.

The term “contacting” as used herein refers to bring or put in contact, to be in or come into contact. The term “contact” as used herein refers to a state or condition of touching or of immediate or local proximity. Contacting to a target destination, such as, but not limited to, an organ, tissue, cell, or tumor, may occur by any means of administration known to the skilled artisan.

As used herein, the terms “providing,” “administering,” and “introducing,” are used interchangeably herein and refer to the placement into a cell, organism, or subject by a method or route which results in at least partial localization to a desired site. Administration can be by any appropriate route which results in delivery to a desired location in the cell, organism, or subject.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### Systems

Disclosed herein are systems for nucleic acid-guided DNA synthesis. In some embodiments, the system comprise a defense-associated reverse transcriptase (DRT), or a nucleic acid encoding thereof, and engineered target nucleic acid comprising one or more sequences of interest, or a nucleic acid encoding thereof.

The system may be a cell free system. Also disclosed is a cell comprising the system described herein. In some embodiments, the cell is a prokaryotic cell. In some embodiments, the cell is a eukaryotic cell.

Defense-associated reverse transcriptases (DRTs) are derived from a class of retroelements with antiphage function, originating from a monophyletic clade of reverse transcriptases termed the Unknown Group (UG). Nine UG subgroups (DRT1-9) show phage defense functions alongside a functional intact RT domain. The DRT may include any enzyme capable of directed reverse transcription from a template as described herein, regardless of which subgroup from which it is derived. In some embodiments, the defense-associated reverse transcriptase (DRT) is from or derived from a DRT2-family phage defense system.

The DRT may be from or derived from any of Vibrio cholerae, Escherichia coli, Klebsiella pneumoniae, Yersinia ruckeri, Vibrio crassostreae, Alloalcanivorax xenomutans, Collimonas arenae, Burkholderia cepacian, Pseudomonas syringae, Stutzerimonas stutzeri, Vibrio splendidus, Vibrio tasmaniensis 1F-267, Stutzerimonas stutzeri XLDN-R, Vibrio metoecus, Collimonas arenae, Vibrio harveyi, [Pseudomonas] sp. BICA1-14, Aureimonas sp. Leaf324, Vibrio atlanticus, Albimonas donghaensis, Pseudomonas resinovorans, Stutzerimonas stutzeri, Vibrio sp. Vb0932, Inquilinus limosus, Bacillus thuringiensis, Bacillus anthracis, Parvibaculum sp., Vibrio chagasii, Photobacterium leiognathi subsp. mandapamensis, Photobacterium damselae, Vibrio pectenicida, Klebsiella pneumoniae, Pseudoxanthomonas composti, Brevundimonas sp., Vibrio parahaemolyticus, Roseobacter sp. TSBP12, Pseudovibrio sp. Tun.PSC04-5.I4, Priestia taiwanensis, Burkholderia cepacia, Dyella sp. ASV21, Fundidesulfovibrio soli, Pseudomonas guariconensis, Vibrio diabolicus, Escherichia coli, Alloalcanivorax xenomutans strain:AGSA2-2, Lederbergia citrea, Paenalkalicoccus suaedae, Shewanella algae, Lysinibacillus capsici, Enterobacter hormaechei, Burkholderia pseudomallei, Thalassobium sp., Pseudomonas sp. WS 5411, Vibrio sp. 708, Pantoea sp., Pectobacterium versatile, Arsenophonus apicola, Enterobacter asburiae, Vibrio cincinnatiensis, Enterobacter kobei, Providencia alcalifaciens R90-1475, Burkholderia ubonensis, Pantoea sp. CCBC3-3-1, Pectobacterium aroidearum, Burkholderia pseudomultivorans, Vibrio sp. SCSIO 43153, Shewanella sp. SM95, Enterobacter roggenkampii, Hafnia paralvei, Rhizobium paranaense, Undibacterium seohonense, Thalassospira xiamenensis, Neisseria elongata subsp. glycolytica ATCC 29315, Cupriavidus taiwanensis, Xanthomonas campestris pv. zingibericola, Stenotrophomonas maltophilia, Xanthomonadales bacterium 14-68-21, Rhizobium sp. CCGE 510, Pseudomonas aeruginosa, Massilia brevitalea, Pelagibacterium sp. SCN 63-126, Litoribrevibacter albus, Alteromonas macleodii, Methyloversatilis discipulorum, Conchiformibius kuhniae DSM 17694, Conchiformibius kuhniae, Agrobacterium sp. MAFF310724, Agrobacterium tumefaciens, Brucella endophytica, Rhizobium lemnae, Allorhizobium taibaishanense, Rhizobium sp. SSM4.3, Janthinobacterium sp. HH107, Pseudomonas sp. S3E12, Bacillus alkalisoli, Bacillus cereus biovar anthracis str. CI, Photobacterium leiognathi, Zoogloea oryzae, Janthinobacterium sp. FT14W, Pandoraea iniqua, Moritella sp., Moritella dasanensis ArB 0140, Lysinibacillus sp. A1, Aeromonas hydrophila, Aeromonas aquatica, Aeromonas bestiarum, Aeromonas caviae, Rhizobium leguminosarum, Rhizobium sp. SEMIA4064, Brucella tritici, Rhizobium lentis, Pseudomonas sp. GD03721, Pseudomonas sp., Pseudomonas corrugata, Pseudomonas sp. GD03985, Stutzerimonas stutzeri A1501, Stutzerimonas nitrititolerans, Pseudomonas migulae, Pseudomonas campi, Paracoccaceae bacterium, Brucella intermedia, Diaphorobacter sp., Rhodobacter capsulatus, Rhodobacteraceae bacterium S2214, Janthinobacterium sp. 67, Aeromonas dhakensis, Duganella sacchari, Halomonas maura, Desulfurivibrio alkaliphilus AHT 2, Vibrio nereis, Azospirillum brasilense, Burkholderia sp. B21-005, Paraglaciecola sp. T6c, Burkholderia sp. 9120, Idiomarina ramblicola, Paraburkholderia fungorum, Pseudomonas savastanoi, Pseudomonas amygdali pv. loropetali, Neisseria subflava, Sulfitobacter sabulilitoris, Brevundimonas huaxiensis, Vibrio toranzoniae, Rhizobium glycinendophyticum, Rhizobium ruizarguesonis, Sinorhizobium saheli, Novacetimonas cocois, Flavonifractor sp. An100, Ignatzschineria cameli, Ignatzschineria indica, Bartonella sp. M0283, Neisseria dumasiana, Spongiibacter pelagi, uncultured Zhongshania sp., Hansschlegelia plantiphila, Alcanivorax sp., Rheinheimera sp., Sphingomonas pollutisoli, Yersinia ruckeri, Yersinia pseudotuberculosis, Yersinia aleksiciae, Bacillus inaquosorum, Klebsiella sp. ZYC-1, Aeromonas media, Mitsuokella multacida, Pseudomonas sp. BLCC-B13, Pseudomonas sp. Gutcm_11s, Shewanella sp. SM68, Shewanella baltica OS183, Pseudomonas mendocina DLHK, Bacillus velezensis, Pseudomonas aeruginosa DQ8, Pseudomonas sp. BAY1663, Bacillus mojavensis, Alkalihalobacillus pseudalcaliphilus, Lysinibacillus sp. LK3, Bacillus gobiensis, Agarivorans gilvus, Legionella quinlivanii DSM 21216, Marinomonas sp. SBI8L, Clostridiales bacterium KLE1615, Devosia elaeis, Aliivibrio fischeri, Lysinibacillus sp. AR18-8, Sutcliffiella halmapala, Paracoccus sediminis, Pseudomonas sp. FEMGT703P, Minwuia thermotolerans, Stutzerimonas kunmingensis, Ruegeria sp. A3M17, Blautia sp. TF12-12AT, Aliivibrio sp. EL58, Colwellia sp. Arc7-635, Oxalobacteraceae bacterium, Pseudoalteromonas sp. S4741, Pseudoalteromonas aurantia, Cytobacillus solani, Bradyrhizobium daqingense, Pseudomonas profundi, Pseudooceanicola albus, Pseudomonas nitroreducens, Shewanella sp. ISTPL2, Pseudomonas ceruminis, Pseudoalteromonas sp. MT33b, Pseudomonas vanderleydeniana, Pseudomonas taiwanensis, Pseudoalteromonas sp. APC 3215, Zobellella iuensis, Parahaliea mediterranea, Pseudomonas sp. PNP, Bacillus subtilis, Oceanimonas baumannii, Vibrio kanaloae, Klebsiella aerogenes, Aeromonas jandaei, Pantoea ananatis, Collimonas humicola, Rheinheimera oceanensis, Polymorphobacter sp. PAMC 29334, Bacillus atrophaeus, Phycisphaeraceae bacterium, Klebsiella michiganensis E718, Paraburkholderia tagetis, Vibrio furnissii, Ruminococcus sp. AF17-11, Cupriavidus basilensis OR16, Acinetobacter baumannii, Pseudomonas syringae pv. actinidiae ICMP 19072, Pectobacterium brasiliense, Flavonifractor plautii, Allomuricauda eckloniae, Duganella sp. CF517, Vibrio sp., Aeromonas veronii, Acinetobacter pittii, Tsuneonella flava, Photobacterium phosphoreum, Photobacterium kishitanii, Photobacterium sp. GB-50, Acinetobacter haemolyticus, Paraburkholderia sp. BL6669N2, Chromatocurvus halotolerans, Altericroceibacterium spongiae, Paraburkholderia sp. RAU2J, Burkholderia stabilis, Nitrincola iocasae, Klebsiella michiganensis, Marinobacter antarcticus, Rugamonas fusca, Oceanobacillus sp. J11TS1, Burkholderia sp. CpTa8-5, Sphingomonas ursincola, Paraburkholderia caribensis, Rhizobium anhuiense bv. trifolii, Gluconobacter morbifer G707, Cronobacter sakazakii, Lactobacillus intestinalis, Vibrio cyclitrophicus FF160, Vibrio fluvialis, Vibrio sp. JCM 18905, Chrysiogenes arsenatis DSM 11915, Pseudomonas rhodesiae, Photobacterium angustum, Yersinia similis, Vibrio sp. MEBiC08052, Photobacterium aquimaris, Vibrio natriegens, Ignavibacteria bacterium RBG_16_35_7, Bradyrhizobium sp. NFR13, Cellvibrio sp. PSBB023, Verrucomicrobia bacterium ADurb.Bin118, Serratia marcescens, Psychromonas sp. Urea-02u-13, Vibrio cyclitrophicus, Lysinibacillus sphaericus, Sphingomonas sp. UV9, Mesorhizobium composti, Vibrio genomo sp. F6, Rheinheimera tangshanensis, Leclercia adecarboxylata, Devosia sp. Leaf420, Duganella qianjiadongensis, Caenispirillum salinarum AK4, Nitrogeniibacter mangrovi, Pantoea multigeneris, Rhizobium leguminosarum bv. viciae, Burkholderia cenocepacia, Klebsiella grimontii, Gluconobacter sp. R75690, Pantoea sp. SM3640, Pantoea ananatis LMG 5342, Sulfitobacter sp. M74, Acinetobacter baylyi TG19579, Hyphomonadaceae bacterium UKL13-1, Acinetobacter soli, Sphaerotilus montanus, Proteus sp. G2669, Morganella morganii, Pseudomonas otitidis, Actinobacillus pleuropneumoniae, Pseudidiomarina donghaiensis, Idiomarina loihiensis, Albimonas pacifica, Mannheimia haemolytica D171, Mannheimia haemolytica, Pectobacterium colocasium, Pectobacterium sp. PL152, Bacillus cereus, Neisseria weaveri, Aeromonas sp. QDB63, Neisseria sp. HMSC70E02, Agrobacterium larrymoorei, Paracoccus sediminicola, Pseudooceanicola sp. HF7, Paracoccus alkenifer, Paracoccus pantotrophus J46, Teredinibacter turnerae T8402, Eubacterium sp. 1001713B170207_170306_E7, Lichenibacterium sp. 6Y81, Alishewanella jeotgali KCTC 22429, Vibrio paucivorans, Acinetobacter ursingii, Acinetobacter sp. WCHA39, Pantoea sp. PSNIHI1, Verrucomicrobiota bacterium, Wohlfahrtiimonas chitiniclastica, Photobacterium damselae subsp. damselae, Exiguobacterium sp. ERU653, Rhizobium azibense, Salinicoccus cyprini, Psychrobacillus soli, Chromobacterium amazonense, Agrobacterium sp. YIC 4121, Photorhabdus namnaonensis, Providencia rustigianii, Chitinimonas sp. BJYL2, Plesiomonas shigelloides, Acinetobacter modestus, Larkinella punicea, Alphaproteobacteria bacterium HGW-Alphaproteobacteria-2, Telluria aromaticivorans, Neisseria zalophi, Bacillus subtilis subsp. subtilis, Acinetobacter johnsonii, Vibrio paracholerae HE-16, Vibrio harveyi NBRC 15634=ATCC 14126=KCTC 12724, Vibrio alginolyticus, Vibrio sp. T3Y01, Klebsiella pneumoniae subsp. pneumoniae 1084, Pseudomonas putida, Klebsiella pneumoniae subsp. pneumoniae NTUH-K2044, Arsenophonus endosymbiont of Apis mellifera, Vibrio campbellii HY01, Vibrio owensii, Vibrio jasicida, Klebsiella pneumoniae subsp. pneumoniae, Aeromonas sp. QDB39, Pseudomonas sp. GD03919, Pseudomonas sp. GD03875, Neisseria flavescens NRL30031/H210, Pseudomonas syringae pv. castaneae, Vibrio vulnificus, Neisseria elongata subsp. glycolytica, Neisseria flavescens, uncultured Alcanivorax sp., Alloalcanivorax xenomutans, Nitratidesulfovibrio vulgaris DP4, Pseudomonas sp. GD04042, Pseudomonas kuykendallii, Vibrio sp. G41H, Vibrio sp. ZF 223, Pseudoalteromonas sp. 2CM28B, Pseudoalteromonas sp. S3260, Pseudoalteromonas sp. bablab_jr011, Klebsiella variicola, Vibrio parahaemolyticus S152, Vibrio antiquarius, Pseudomonas syringae pv. actinidiae ICMP 19073, Pseudomonas syringae pv. actinidiae ICMP 19071, Photobacterium leiognathi lrivu.4.1, Pseudomonas parafulva NBRC 16636=DSM 17004, Pseudomonas syringae pv. actinidiae, Burkholderia pseudomallei MSHR7527, Vibrio casei, Pseudomonas syringae pv. cerasicola, Pseudomonas amygdali pv. morsprunorum, Aeromonas sp. FDAARGOS 1408, Paraburkholderia sp. F2, Shewanella sp. SM32, Pseudomonas chlororaphis, Cronobacter sakazakii NCEVIB 8272, Pantoea stewartii, Pseudomonas amygdali pv. photiniae, Klebsiella quasipneumoniae, Raoultella terrigena, Burkholderia contaminans, Pseudoalteromonas sp. TB41, Proteus sp. FME41, Proteus hauseri, Aeromonas sp. QDB68, Providencia alcalifaciens Dmel2, Rhizobium sp. BR 318, Devosia sp. 63-57, Bacillus pacificus, Bacillus cereus group sp. BcHK28, Lysinibacillus fusiformis, Rhodobacter capsulatus Y262, Lysinibacillus fusiformis ZB2, Altererythrobacter sp. FM1, Rhizobium laguerreae, Paracoccus pantotrophus, Exiguobacterium marinum DSM 16307, Agrobacterium tumefaciens LBA4213 (Ach5), Agrobacterium deltaense RV3, Rhodobacter capsulatus DE442, Rhodobacter capsulatus SB 1003, and Acinetobacter nosocomialis. In some embodiments, the DRT is from or derived from Klebsiella pneumoniae.

In some embodiments, the DRT comprises an amino acid sequence having at least 70% (e.g., at least 75%, at least 80%, at least 85%, at least 90%, at least 95%, at least 96%, at least 97%, at least 98%, at least 99%, or more) identity with any of SEQ ID NOs: 1-824. In some embodiments, the DRT comprises an amino acid sequence any of SEQ ID NOs: 1-824.

Any of the proteins described or referenced herein may comprise one or more amino acid substitutions as compared to the recited sequences. An amino acid “replacement” or “substitution” refers to the replacement of one amino acid at a given position or residue by another amino acid at the same position or residue within a polypeptide sequence. Amino acids are broadly grouped as “aromatic” or “aliphatic.” An aromatic amino acid includes an aromatic ring. Examples of “aromatic” amino acids include histidine (H or His), phenylalanine (F or Phe), tyrosine (Y or Tyr), and tryptophan (W or Trp). Non-aromatic amino acids are broadly grouped as “aliphatic.” Examples of “aliphatic” amino acids include glycine (G or Gly), alanine (A or Ala), valine (V or Val), leucine (L or Leu), isoleucine (I or He), methionine (M or Met), serine (S or Ser), threonine (T or Thr), cysteine (C or Cys), proline (P or Pro), glutamic acid (E or Glu), aspartic acid (A or Asp), asparagine (N or Asn), glutamine (Q or Gin), lysine (K or Lys), and arginine (R or Arg).

The amino acid replacement or substitution can be conservative, semi-conservative, or non-conservative. The phrase “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz and Schirmer, Principles of Protein Structure, Springer-Verlag, New York (1979)). According to such analyses, groups of amino acids may be defined where amino acids within a group exchange preferentially with each other, and therefore resemble each other most in their impact on the overall protein structure (Schulz and Schirmer, supra). Examples of conservative amino acid substitutions include substitutions of amino acids within the sub-groups described above, for example, lysine for arginine and vice versa such that a positive charge may be maintained, glutamic acid for aspartic acid and vice versa such that a negative charge may be maintained, serine for threonine such that a free —OH can be maintained, and glutamine for asparagine such that a free —NH₂ can be maintained. “Semi-conservative mutations” include amino acid substitutions of amino acids within the same groups listed above, but not within the same sub-group. For example, the substitution of aspartic acid for asparagine, or asparagine for lysine, involves amino acids within the same group, but different sub-groups. “Non-conservative mutations” involve amino acid substitutions between different groups, for example, lysine for tryptophan, or phenylalanine for serine, etc.

The engineered target nucleic acid(s) comprise one or more sequences of interest (e.g., a sequence which includes the template for a desired DNA product). The sequences of interest may be any desired sequence. The sequence of interest may comprise or encode a product useful in genome editing, biological research, molecular recording, and nucleic acid sequencing technologies. In some embodiments, the sequence of interest includes or encodes one or more sequences to process the product or downstream RNA or protein products into fragments (e.g., monomers of a concatenated product). Exemplary processing sequences include self-cleaving deoxyribozyme sequences, or recognition sequences for a nuclease such as a restriction enzyme or Cas9, a self-cleaving ribozyme sequence, or a T2A or P2A peptide sequence for programmed ribosomal skipping.

The engineered target nucleic acid(s) may be comprised of DNA, RNA, or a DNA/RNA hybrid thereof. In some embodiments, the engineered target nucleic acid(s) is RNA.

In some embodiments, the engineered target nucleic acid(s) comprise at least one chemical modification or chemically modified base or nucleoside. The chemical modifications may comprise any modification which is or is not present in naturally occurring forms of adenosine, guanosine, uridine, thymidine, or cytidine ribonucleosides or deoxyribnucleosides. For example, an engineered target nucleic acid may include both naturally occurring and non-naturally occurring modifications. Chemical modifications may be located in any portion of the engineered target nucleic acid and the engineered target nucleic acid may contain any percentage of modified nucleosides (1-100%). A particular modification may be used for every particular type of nucleoside or base (e.g., every uridine is modified to a 1-methyl-pseudouridine) or on a per base level.

In some embodiments, the at least one chemical modification comprises a modified uridine residue. Exemplary nucleosides having a modified uracil include pseudouridine (Ψ), pyridin-4-one nucleoside, 5-aza-uridine, 6-aza-uridine, 2-thio-5-aza-uridine, 2-thio-uridine, 4-thio-uridine, 4-thio-pseudouridine, 2-thio-pseudouridine, 5-hydroxy-uridine, 5-aminoallyl-uridine, 5-halo-uridine (e.g., 5-iodo-uridine or 5-bromo-uridine), 3-methyl-uridine, 5-methyl-uridine, 5-methoxy-uridine, uridine 5-oxyacetic acid, uridine 5-oxyacetic acid methyl ester, 5-carboxymethyl-uridine, 1-carboxymethyl-pseudouridine, 5-carboxyhydroxymethyl-uridine, 5-carboxyhydroxymethyl-uridine methyl ester, 5-methoxycarbonylmethyl-uridine, 5-methoxycarbonylmethyl-2-thio-uridine, 5-aminomethyl-2-thio-uridine, 5-methylaminomethyl-uridine, 5-methylaminomethyl-2-thio-uridine, 5-methylaminomethyl-2-seleno-uridine, 5-carbamoylmethyl-uridine, 5-carboxymethylaminomethyl-uridine, 5-carboxymethylaminomethyl-2-thio-uridine, 5-propynyl-uridine, 1-propynyl-pseudouridine, 5-taurinomethyl-uridine, 1-taurinomethyl-pseudouridine, 5-taurinomethyl-2-thio-uridine, 1-taurinomethyl-4-thio-pseudouridine, 1-methylpseudouridine, 5-methyl-2-thio-uridine, 1-methyl-4-thio-pseudouridine, 4-thio-1-methyl-pseudouridine, 3-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 5,6-dihydrouridine, 5-methyl-dihydrouridine, 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, 1-methylpseudouridine, 3-(3-amino-3-carboxypropyl)uridine, 1-methyl-3-(3-amino-3-carboxypropyl)pseudouridine, 5-(isopentenylaminomethyl)uridine, 5-(isopentenylaminomethyl)-2-thio-uridine, α-thio-uridine, 2′-O-methyl-uridine, 5,2′-O-dimethyl-uridine, 2′-O-methyl-pseudouridine, 2-thio-2′-O-methyl-uridine, 5-methoxycarbonylmethyl-2′-O-methyl-uridine, 5-carbamoylmethyl-2′-O-methyl-uridine, 5-carboxymethylaminomethyl-2′-O-methyl-uridine, 3,2′-O-dimethyl-uridine, 5-(isopentenylaminomethyl)-2′-O-methyl-uridine, 1-thio-uridine, deoxythymidine, 2′-F-ara-uridine, 2′-F-uridine, 2′-OH-ara-uridine, 5-(2-carbomethoxyvinyl) uridine, and 5-[3-(1-E-propenylamino)uridine.

In some embodiments, the at least one chemical modification comprises a modified thymidine residue. Exemplary nucleosides having a modified thymidine include allyamino-thymidine, aza thymidine, deaza thymidine, deoxy-thymidine, and 2-thiothymidine.

In some embodiments, the at least one chemical modification comprises a modified cytosine residue. Exemplary nucleosides having a modified cytosine include 5-aza-cytidine, 6-aza-cytidine, pseudoisocytidine, 3-methyl-cytidine, N4-acetyl-cytidine, 5-formyl-cytidine, N4-methyl-cytidine, 5-methyl-cytidine, 5-halo-cytidine, 5-hydroxymethyl-cytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine, α-thio-cytidine, 2′-O-methyl-cytidine, 5,2′-O-dimethyl-cytidine, N4-acetyl-2′-O-methyl-cytidine, N4,2′-O-dimethyl-cytidine, 5-formyl-2′-O-methyl-cytidine, N4,N4,2′-O-trimethyl-cytidine, 1-thio-cytidine, 2′-F-aracytidine, 2′-F-cytidine, and 2′-OH-aracytidine.

In some embodiments, the at least one chemical modification comprises a modified adenine residue. Exemplary nucleosides having a modified adenine include 2-amino-purine, 2,6-diaminopurine, 2-amino-6-halo-purine, 6-halo-purine, 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine, 2-methyl-adenine, N6-methyl-adenosine, 2-methylthio-N6-methyl-adenosine, N6-isopentenyl-adenosine, 2-methylthio-N6-isopentenyl-adenosine, N6-(cis-hydroxyisopentenyl)adenosine, 2-methylthio-N6-(cis-hydroxyisopentenyl)adenosine, N6-glycinylcarbamoyl-adenosine, N6-threonylcarbamoyl-adenosine, N6-methyl-N6-threonylcarbamoyl-adenosine, 2-methylthio-N6-threonylcarbamoyl-adenosine, N6,N6-dimethyl-adenosine, N6-hydroxynoryalylcarbamoyl-adenosine, 2-methylthio-N6-hydroxynoryalylcarbamoyl-adenosine, N6-acetyl-adenosine, 7-methyl-adenine, 2-methylthio-adenine, 2-methoxy-adenine, α-thio-adenosine, 2′-O-methyl-adenosine, N6,2′-O-dimethyl-adenosine, N6,N6,2′-O-trimethyl-adenosine, 1,2′-O-dimethyl-adenosine, 2′-O-ribosyladenosine (phosphate), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

In some embodiments, the at least one chemical modification comprises a modified guanine residue. Exemplary nucleosides having a modified guanine include inosine, 1-methyl-inosine, wyosine, methylwyosine, 4-demethyl-wyosine, isowyosine, wybutosine, peroxywybutosine, hydroxywybutosine, undermodified hydroxywybutosine, 7-deaza-guanosine, queuosine, epoxyqueuosine, galactosyl-queuosine, mannosyl-queuosine, 7-cyano-7-deaza-guanosine, 7-aminomethyl-7-deaza-guanosine, archaeosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methyl-inosine, 6-methoxy-guanosine, 1-methyl-guanosine, N2-methyl-guanosine, N2,N2-dimethyl-guanosine, N2,7-dimethyl-guanosine, N2,N2,7-dimethyl-guanosine, 8-oxo-guanosine, 7-methyl-8-oxo-guanosine, 1-methyl-6-thio-guanosine, N2-methyl-6-thio-guanosine, N2,N2-dimethyl-6-thio-guanosine, α-thio-guanosine, 2′-O-methyl-guanosine, N2-methyl-2′-O-methyl-guanosine, N2,N2-dimethyl-2′-O-methyl-guanosine, 1-methyl-2′-O-methyl-guanosine, N2,7-dimethyl-2′-O-methyl-guanosine, 2′-O-methyl-inosine, 1,2′-O-dimethyl-inosine, and 2′-O-ribosylguanosine (phosphate).

The engineered target nucleic acid(s) can be derived from endogenous sequences (e.g., endogenous ncRNA sequences). For example, the engineered target nucleic acid may comprise one or more modifications (nucleotide substitutions, additions, deletions, and combinations thereof) as compared to an endogenous sequence, for example, to include one or more sequences of interest.

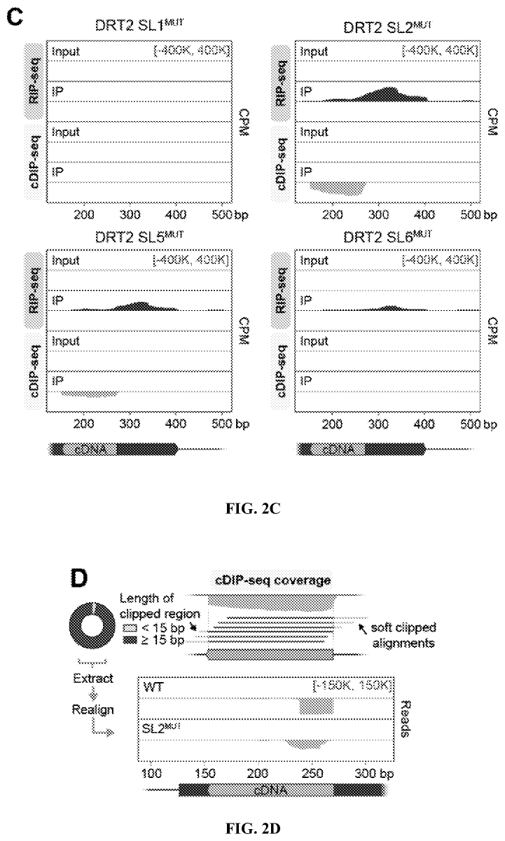

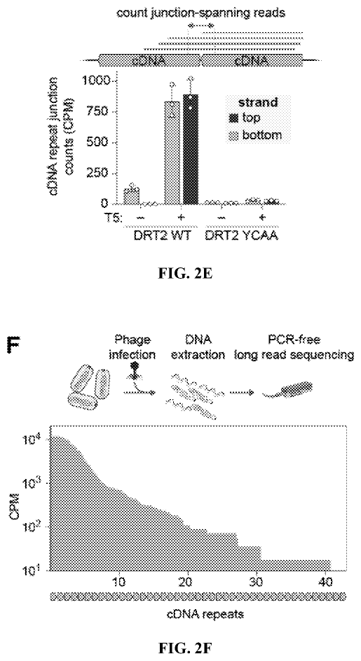

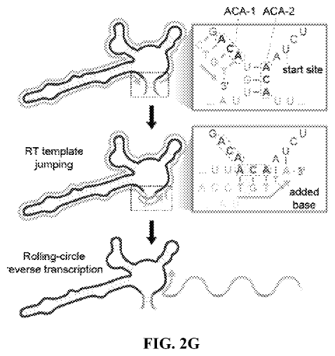

The engineered target nucleic acid may comprise any secondary structure and sequence which allows the DRT to recognize the sequences of interest and produce a DNA product. The engineered target nucleic acid may include any number of stem-loop (SL) elements within each region of the engineered target nucleic acid, see for example FIG. 2A.

The engineered target nucleic acid(s) include a template region used as the template for transcription of the DNA product. The template region may be flanked by a 5′ region and a 3′ region. The template region may be any size corresponding to the desired sequence of interest. In some embodiments, the template region is greater than 10 nucleotides (e.g., greater than 25 nucleotides, greater than 50 nucleotides, greater than 100 nucleotides, greater than 150 nucleotides, greater than 200 nucleotides, greater than 250 nucleotides, greater than 300 nucleotides, greater than 400 nucleotides or greater than 500 nucleotides).

In some embodiments, the engineered target nucleic acid(s) comprise a template region comprising the one or more sequence(s) of interest, which are used to generate the desired DNA product, or a segment thereof. The template region may comprise one or more loops (e.g., internal loops, hairpin loops, multi-branch loops (multiloops), terminal loops), bulges, stacks, junctions, and combinations thereof.

In some embodiments, the template is abutting a short basal stem. The short basal stem may be 2 to 10 nucleotides in length. The short basal stem may be 2 to 5 nucleotides in length. In some embodiments, the short basal stem is 2 nucleotides in length. In some embodiments, the short basal stem is 3 nucleotides in length. In some embodiments, the short basal stem is 4 nucleotides in length. In some embodiments, the short basal stem is 5 nucleotides in length.

In some embodiments, the 5′ end of the template sequence is homologous to the sequence adjacent to the 3′ of the template sequence. In some embodiments, the 5′ end of the template sequence is homologous to the sequence at the 3′ end of the template region in the short basal stem. In select embodiments, the 5′ end of the template sequence and the sequence adjacent to the 3′ of the template sequence is ACA or GGA. In some embodiments, the sequence immediately 5′ to the template sequence, corresponding to the sequence in the basal stem which hybridizes at least partially to the sequence homologous to the 5′ end of the template sequence, comprises a sequence enriched for G and U. This sequence may be derived from an endogenous ncRNA sequence (e.g., a ncRNA sequence associated with the desired DRT).

The one or more sequence(s) of interest may be any sequence corresponding to the complement of the desired DNA product. For example, the sequences of interest may be reverse transcribed as or result in a product of: donor DNA sequences useful in homologous recombination based methods and as inserts for gene editing; aptamers; inputs for DNA libraries; nucleic acid binding sequences for other agents (e.g., proteins, small regulatory nucleic acids); and/or encode proteins or other nucleic acid products.

In some embodiments, the engineered target nucleic acid(s) comprise a 3′ region. The 3′ region extends from the 3′ end of the short basal stem forming the template region to the 3′ end of the engineered target nucleic acid. In some embodiments, the 3′ region comprises a scaffold for the recruitment of the DRT. In some embodiments, the 3′ region comprises one or more stem-loops. In some embodiments, the 3′ region comprises two or more stem-loops consistent in structure with an endogenous ncRNA sequence (e.g., a ncRNA sequence associated with the desired DRT).

In some embodiments, the 3′ region comprises a series of (e.g., two, three, or more) 3′ stem-loops. In some embodiments, the 3′ region comprises the series of 3′ stem-loops about 10 or more nucleotides from the 3′ end of the short basal stem. In some embodiments, each of the stem-loops in the series of stem-loops is separated by 2 or more nucleotides. In select embodiments, each of the stem-loops is separated by 2-10 nucleotides from the adjacent stem-loops. In some embodiments, the 3′ end of the ncRNA has one or more additional nucleotides following the most 3′ stem-loop structure.

In some embodiments, the engineered target nucleic acid(s) comprise a 5′ region. The 5′ region is the region of the engineered target nucleic acid 5′ of the template region. In some embodiments, the 5′ region comprises at least one stem-loop. In select embodiments, the 5′ region comprises a single stem-loop. In some embodiments, the at least one or single step loop is separated from the short basal stem by one or more (e.g., 1, 2, 3, 4, 5, 6, 7, 8, 9, 10 or more) nucleotides.

The present disclosure also encompasses engineered target nucleic acids as described herein and compositions comprising thereof. The present disclosure also encompasses nucleic acids encoding or comprising the engineered target nucleic acids as described herein and compositions comprising thereof.

In some embodiments, the nucleic acids encoding or comprising the engineered target nucleic acids include sequences or structures allowing the engineered target nucleic acid to be produced in the cell or system from the nucleic acid. For example, the nucleic acids encoding or comprising the engineered target nucleic acids may include ribozymes or other processing means which allow precise generation of boundaries of the engineered target nucleic acid. In some embodiments, the nucleic acids encoding or comprising the engineered target nucleic acids may include a poly-T termination sequence was encoded immediately downstream of the 3′ end of the engineered target nucleic acid. In some embodiments, the sequences used for the nucleic acids encoding or comprising the engineered target nucleic acids may include one or more sequences derived from an endogenous sequence which encodes endogenous ncRNA sequences.

The systems may further comprise other components necessary for DNA synthesis, including for example, dNTPs, RNase inhibitors, buffers, etc.

The systems may further comprise one or more genomic engineering reagents selected from: single-stranded annealing proteins (SSAP), guide RNAs, DNA endonucleases, ribonucleases, transcriptional activators, transcriptional repressors, histone-modifying proteins, integrases, recombinases, DNA polymerases, and combinations thereof. As described below, in some embodiments, the one or more genomic engineering reagents may be provided as a protein conjugate with the DRT.

### Protein Conjugates

Also disclosed herein are protein conjugates comprising a defense-associated reverse transcriptase (DRT). As used herein, “conjugate” refers to the linking of two or more moieties or molecules to each other by covalent or non-covalent interactions. More specifically, the terms “protein conjugate” refer to a protein (e.g., a DRT as disclosed herein) that has been modified by the addition of another moiety or molecule (e.g., another peptide, protein, or polypeptide as in an “antibody-peptide conjugate”).

The other moiety or molecule may include, but is not limited to: DNA-targeting proteins and/or nuclease proteins (zinc finger proteins/nucleases; TALEs or TALE nucleases; Cas nucleases, nickases, or catalytically inactive nucleases such as dCas9); RNA-binding proteins (e.g., splicing factors, the RNA editing protein ADAR, or polyadenylation proteins); ribonuclease proteins (e.g., ribonuclease H); other effector domains or proteins (e.g., integrases, recombinases, polymerases (e.g., DNA polymerases)); and single-stranded DNA binding proteins (e.g., the phage T5 D11 protein).

Protein conjugates can be linked using standard chemical conjugation techniques. Methods of chemical conjugation of peptides are known in the art. Common conjugation strategies are generally based on side-chain modification of lysine or cysteine. Noncanonical amino acids may facilitate introduction of chemically orthogonal handles at predefined sites in a given protein sequence, e.g., for oxime ligation or click chemistry methods. In addition, a wide variety of methods are available for site-specific protein modification with varying degrees of versatility.

Exemplary chemical linker systems include, but are not limited to, the carbodiimide (EDC), the thiol-maleimide, the succinimidyl 3-(2-pyridyldithio)propionate (SPDP) and the periodate systems. In the carbodiimide system, a water soluble carbodiimide reacts with carboxylic acid groups on proteins and activates the carboxyl group. The carboxyl group is coupled to an amino group of the second protein. The result of this reaction is a noncleavable amide bond between two proteins. In the thiol-maleimide system, a sulfhydryl group is introduced onto an amine group of one of the proteins using a compound such as Traut's reagent. The other protein is reacted with an NHS ester (such as gamma-maleimidobutyric acid NHS ester (GMBS)) to form a maleimide derivative that is reactive with sulfhydryl groups. The two modified proteins are then reacted to form a covalent, noncleavable linkage. SPDP is a heterofunctional crosslinking reagent that introduces aliphatic thiol groups into either moiety. The thiol group reacts with an amine group forming a non-cleavable bond. Periodate coupling utilizes oligosaccharide groups or N-terminal Ser/Thr residues on either the carrier or the protein. If these groups are available, an active aldehyde is formed which can react with an amino, hydrazine, and hydroxylamine group on the other member of the conjugate. For example, active aldehyde groups can be formed on the carbohydrate groups present on antibody molecules. These groups can then be reacted with amino groups on a protein or polypeptide to generate a stable conjugate. Alternatively, the periodate oxidized antibody can be reacted with a hydrazide derivative of a protein or polypeptide which will also yield a stable conjugate. The components of the conjugates may include other moieties or linkers which are attached to the reactive moieties of the chemical linker systems but not required for formation of the bond and conjugate, provided the conjugation reaction can proceed. Accordingly, conjugate may retain the other moieties or linkers to separate the components.

Besides chemical strategies, enzymatic conjugations may be suitable, as they enable highly controlled modification of protein (e.g., antibodies) through specific peptide tags. Exemplary enzymatic processes include, but are not limited to, sortase ligation, subtiligase-catalyzed ligation, phosphopantetheinyl transferase, tyrosinase, and transglutaminase. The components of the conjugates may include other moieties or linkers (e.g., amino acids) to which the substrates for the enzymatic reaction are attached, provided the enzymatic reaction can proceed. Accordingly, conjugate may retain the other moieties or linkers to separate the components.

The protein conjugates can also be produced by using two halves of a known interaction pair (e.g., recruitment systems and binding pairs).

A binding pair refers to a pair of molecules comprising a binding member and a binding partner which have particular specificity for each other and under normal conditions bind to each other in preference to binding to other molecules. The interaction of the binding pair is typically non-covalent, but may also result in formation of a covalent bond. The binding member and binding partner may comprise a part of a larger molecule. The binding pair may include protein:protein binding pairs (e.g., protein:antibody, biotin:avidin), protein:polynucleotide binding pairs, protein:carbohydrate binding pairs, protein:small molecule binding pairs, polynucleotide:polynucleotide binding pairs, and the like. Examples of a specific binding pair include an antibody and an antigen, biotin and avidin or streptavidin, a ligand and a receptor, a lectin and a carbohydrate, an enzyme and a cofactor or substrate, oppositely charged ionic groups, redox/electrochemical groups, a chelating group and its binding partner, or a nucleic acid molecule capable of hybridizing to a complementary nucleic acid sequence.

In some embodiments, the binding pair comprises functional groups that react to form a covalent bond. For example, functional groups that facilitate bioconjugation reactions (e.g., thiol conjugation reactions, amine-modified DNAs with carbonyl functional groups, and the like). For exemplary pairs see Kalia J, Raines R T. Curr Org Chem. 2010; 14(2):138-147, Mukesh Digambar Sonawane, Satish Balasaheb Nimse, Journal of Chemistry, vol. 2016, Article ID 9241378, 19 pages, 2016, and Bioconjugate Techniques, Ed. Hermanson, GT, Academic Press, 1996, Pages 727-728, ISBN 9780123423351, each incorporated herein by reference in its entirety.

The recruitment system can comprise any binding pair. For example, the recruitment system may comprise an aptamer and an aptamer binding protein or domain. The recruitment system may be a so-called split system. Split systems include two or more polypeptide chains that reassemble into an operable fusion protein or protein conjugate upon association of the two binding partners. Split systems include, but are not limited to, intein, MS2, or SunTag based systems.

The protein conjugate can also be produced as a contiguous protein (e.g., a fusion protein) using genetic engineering techniques. The protein can be expressed and purified as a single contiguous protein containing both the DRT and the other moiety or molecule.

In the protein conjugate, the DRT and the other moiety or molecule may be linked in any orientation (e.g., N-terminus to C-terminus or either terminus to an internal site) at any location as long as both can separately function. As such, the conjugate described herein is not limited by the method, location, or orientation of the conjugation. In select embodiments, the N-terminus of the activator of DRT is attached to the C-terminus of the other moiety or molecule. In select embodiments, the C-terminus of the DRT is attached to the N-terminus of other moiety or molecule.

The protein conjugate or fusion protein may comprise a linker. The linker may be a polypeptide although other small molecule and alternative polymer-based linkers are also contemplated.

In some embodiments, the linker is a polypeptide having any of a variety of amino acid sequences. These linkers can be produced by using synthetic linkers to couple the proteins or can be encoded by a nucleic acid sequence encoding the fusion protein. Suitable linkers include polypeptides of between 1 amino acids and 100 amino acids in length, between 1 amino acids and 50 amino acids in length, between 1 amino acids and 40 amino acids in length, between 1 amino acids and 30 amino acids in length, between 1 amino acids and 20 amino acids in length, or between 1 amino acids and 10 amino acids in length. The linking peptides may have virtually any amino acid sequence, bearing in mind that the preferred linkers may have a sequence that results in a generally flexible peptide. The use of small amino acids, such as glycine and alanine, are of use in creating a flexible peptide. The creation of such sequences is routine to those of skill in the art. A variety of different linkers suitable for use, include but are not limited to, glycine-serine polymers, glycine-alanine polymers, and alanine-serine polymers.

The DRT, protein conjugate, or fusion protein may further comprise a localization or signal sequence. The localization or signal sequence may direct the protein conjugate or fusion protein to a certain subcellular location (e.g., the nucleus, mitochondria, lysosomes, plasma membrane, endoplasmic reticulum, peroxisomes, Golgi) or to a certain cellular pathway, e.g., the secretory pathway.

The DRT, protein conjugate, or fusion protein may further comprise a tag. The tag includes any tag useful in identifying the protein conjugate or fusion protein. Exemplary tags include, but are not limited to, an antibody tag (e.g., human influenza hemagglutinin (HA), and the like), antibody-epitope tag (a Myc tag, a VS tag, and the like), fluorescent protein tag (e.g., GFP, YFP, RFP, mNeonGreen, TdTomato, and the like), an affinity purification tag (e.g., a Biotin tag, a His tag, and the like), a stability tag (e.g., degron, chemically stabilized FKBP variants, PEST domain, auxin-inducible degron (AID) tag, small molecule-assisted shutoff (SMASh) tag, degradation tag (dTAG), and the like), and the like.

### Nucleic Acids and Delivery

The present disclosure also provides for nucleic acids encoding the components of the disclosed systems, compositions comprising nucleic acids encoding the components of the disclosed systems, systems comprising nucleic acids encoding the components of the disclosed systems, and vectors containing or encoding these nucleic acids. The vectors may be used to propagate the nucleic acid in an appropriate cell and/or to allow expression from the nucleic acid (e.g., an expression vector). The person of ordinary skill in the art would be aware of the various vectors available for propagation and expression of a nucleic acid sequence.

The present disclosure further provides engineered, non-naturally occurring vectors and vector systems, which can encode one or more of the peptides or components of the present systems. The vector(s) can be introduced into a cell that is capable of expressing the polypeptide encoded thereby, including any suitable prokaryotic or eukaryotic cell.

The vectors of the present disclosure may be delivered to a eukaryotic cell in a subject. Modification of the eukaryotic cells via the present system can take place in a cell culture, where the method comprises isolating the eukaryotic cell from a subject prior to the modification. In some embodiments, the method further comprises returning said eukaryotic cell and/or cells derived therefrom to the subject.

Viral and non-viral based gene transfer methods can be used to introduce nucleic acids encoding the disclosed polypeptides or components of the present system into cells, tissues, or a subject. Such methods can be used to administer nucleic acids encoding the disclosed polypeptides or components of the present system to cells in culture, or in a host organism. Non-viral vector delivery systems include DNA plasmids, cosmids, RNA (e.g., a transcript of a vector described herein), nucleic acids, and nucleic acids complexed with a delivery vehicle. Viral vector delivery systems include DNA and RNA viruses, which have either episomal or integrated genomes after delivery to the cell. Viral vectors include, for example, retroviral, lentiviral, adenoviral, adeno-associated and herpes simplex viral vectors.

In certain embodiments, plasmids that are non-replicative, or plasmids that can be cured by high temperature may be used, such that any or all of the necessary components of the system may be removed from the cells under certain conditions. For example, this may allow for DNA integration by transforming bacteria of interest, but then being left with engineered strains that have no memory of the plasmids or vectors used for the integration. Drug selection strategies may be adopted for positively selecting for cells. A donor nucleic acid may contain one or more drug-selectable markers within the cargo. Then presuming that the original donor plasmid is removed, drug selection may be used to enrich for integrated clones. Colony screenings may be used to isolate clonal events.

A variety of viral constructs may be used to deliver the disclosed polypeptides or components of the present system (e.g., DRTs, ncRNA templates) to the targeted cells and/or a subject. Nonlimiting examples of such recombinant viruses include recombinant adeno-associated virus (AAV), recombinant adenoviruses, recombinant lentiviruses, recombinant retroviruses, recombinant herpes simplex viruses, recombinant poxviruses, phages, etc. The present disclosure provides vectors capable of integration in the host genome, such as retrovirus or lentivirus. See, e.g., Ausubel et al., Current Protocols in Molecular Biology, John Wiley & Sons, New York, 1989; Kay, M. A., et al., 2001 Nat. Medic. 7(1):33-40; and Walther W. and Stein U., 2000 Drugs, 60(2): 249-71, incorporated herein by reference.

In one embodiment, a nucleic acid encoding the disclosed polypeptides or components of the present system is contained in a plasmid vector that allows expression of the disclosed polypeptides or components of the present system and subsequent isolation and purification of from the recombinant vector. Accordingly, the disclosed polypeptides or components of the present system disclosed herein can be purified following expression, obtained by chemical synthesis, or obtained by recombinant methods.

To construct cells that express the disclosed polypeptides or components of the present system, expression vectors for stable or transient expression of the disclosed polypeptides or components of the present system may be constructed via conventional methods as described herein and introduced into host cells. For example, nucleic acids encoding the components of the disclosed polypeptides or components of the present system may be cloned into a suitable expression vector, such as a plasmid or a viral vector in operable linkage to a suitable promoter. The selection of expression vectors/plasmids/viral vectors should be suitable for integration and replication in eukaryotic cells.

In certain embodiments, vectors of the present disclosure can drive the expression of one or more sequences in prokaryotic cells. Promoters that may be used include T7 RNA polymerase promoters, constitutive E. coli promoters, and promoters that could be broadly recognized by transcriptional machinery in a wide range of bacterial organisms. The system may be used with various bacterial hosts.

In certain embodiments, vectors of the present disclosure can drive the expression of one or more sequences in mammalian cells using a mammalian expression vector. Examples of mammalian expression vectors include pCDM8 (Seed, Nature (1987) 329:840, incorporated herein by reference) and pMT2PC (Kaufman, et al., EMBO J. (1987) 6:187, incorporated herein by reference). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL. 2nd eds., Cold Spring Harbor Laboratory, Cold Spring Harbor Laboratory Press, Cold Spring Harbor, N.Y., 1989, incorporated herein by reference.

Vectors of the present disclosure can comprise any of a number of promoters known to the art, wherein the promoter is constitutive, regulatable or inducible, cell type specific, tissue-specific, or species specific. In addition to the sequence sufficient to direct transcription, a promoter sequence of the invention can also include sequences of other regulatory elements that are involved in modulating transcription (e.g., enhancers, Kozak sequences and introns). Many promoter/regulatory sequences useful for driving constitutive expression of a gene are available in the art and include, but are not limited to, for example, CMV (cytomegalovirus promoter), EF1a (human elongation factor 1 alpha promoter), SV40 (simian vacuolating virus 40 promoter), PGK (mammalian phosphoglycerate kinase promoter), Ubc (human ubiquitin C promoter), human beta-actin promoter, rodent beta-actin promoter, CBh (chicken beta-actin promoter), CAG (hybrid promoter contains CMV enhancer, chicken beta actin promoter, and rabbit beta-globin splice acceptor), TRE (Tetracycline response element promoter), H1 (human polymerase III RNA promoter), U6 (human U6 small nuclear promoter), and the like. Additional promoters that can be used for expression of the components of the present system, include, without limitation, cytomegalovirus (CMV) intermediate early promoter, a viral LTR such as the Rous sarcoma virus LTR, HIV-LTR, HTLV-1 LTR, Maloney murine leukemia virus (MMLV) LTR, myeoloproliferative sarcoma virus (MPSV) LTR, spleen focus-forming virus (SFFV) LTR, the simian virus 40 (SV40) early promoter, herpes simplex tk virus promoter, elongation factor 1-alpha (EF1-α) promoter with or without the EF1-α intron. Additional promoters include any constitutively active promoter. Alternatively, any regulatable promoter may be used, such that its expression can be modulated within a cell.

Moreover, inducible and tissue specific expression of a RNA, transmembrane proteins, or other proteins can be accomplished by placing the nucleic acid encoding such a molecule under the control of an inducible or tissue specific promoter/regulatory sequence. Examples of tissue specific or inducible promoter/regulatory sequences which are useful for this purpose include, but are not limited to, the rhodopsin promoter, the MMTV LTR inducible promoter, the SV40 late enhancer/promoter, synapsin 1 promoter, ET hepatocyte promoter, GS glutamine synthase promoter and many others. Various commercially available ubiquitous as well as tissue-specific promoters and tumor-specific are available, for example from InvivoGen. In addition, promoters which are well known in the art can be induced in response to inducing agents such as metals, glucocorticoids, tetracycline, hormones, and the like, are also contemplated for use with the invention. Thus, it will be appreciated that the present disclosure includes the use of any promoter/regulatory sequence known in the art that is capable of driving expression of the desired protein operably linked thereto.

The vectors of the present disclosure may direct expression of the nucleic acid in a particular cell type (e.g., tissue-specific regulatory elements are used to express the nucleic acid). Such regulatory elements include promoters that may be tissue specific or cell specific. The term “tissue specific” as it applies to a promoter refers to a promoter that is capable of directing selective expression of a nucleotide sequence of interest to a specific type of tissue (e.g., seeds) in the relative absence of expression of the same nucleotide sequence of interest in a different type of tissue. The term “cell type specific” as applied to a promoter refers to a promoter that is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue. Cell type specificity of a promoter may be assessed using methods well known in the art, e.g., immunohistochemical staining.

Additionally, the vector may contain, for example, some or all of the following: a selectable marker gene, such as the neomycin gene for selection of stable or transient transfectants in host cells; enhancer/promoter sequences from the immediate early gene of human CMV for high levels of transcription; transcription termination and RNA processing signals from SV40 for mRNA stability; 5′- and 3′-untranslated regions for mRNA stability and translation efficiency from highly-expressed genes like α-globin or β-globin; SV40 polyoma origins of replication and ColE1 for proper episomal replication; internal ribosome binding sites (IRESes), versatile multiple cloning sites; T7 and SP6 RNA promoters for in vitro transcription of sense and antisense RNA; a “suicide switch” or “suicide gene” which when triggered causes cells carrying the vector to die (e.g., HSV thymidine kinase, an inducible caspase such as iCasp9), and reporter gene for assessing expression of the chimeric receptor. Suitable vectors and methods for producing vectors containing transgenes are well known and available in the art. Selectable markers also include chloramphenicol resistance, tetracycline resistance, spectinomycin resistance, streptomycin resistance, erythromycin resistance, rifampicin resistance, bleomycin resistance, thermally adapted kanamycin resistance, gentamycin resistance, hygromycin resistance, trimethoprim resistance, dihydrofolate reductase (DHFR), GPT; the URA3, HIS4, LEU2, and TRP1 genes of S. cerevisiae.

When introduced into the cell, the vectors may be maintained as an autonomously replicating sequence or extrachromosomal element or may be integrated into host DNA.

The disclosed polypeptides or components of the present system (e.g., proteins, polynucleotides encoding these proteins, donor polynucleotides and compositions comprising the proteins and/or polynucleotides described herein) may be delivered by any suitable means. In certain embodiments, the polypeptides or system is delivered in vivo. In other embodiments, the polypeptides or system is delivered to isolated/cultured cells (e.g., autologous iPS cells) in vitro to provide modified cells useful for in vivo delivery to patients afflicted with a disease or condition.

Vectors according to the present disclosure can be transformed, transfected, or otherwise introduced into a wide variety of cells. Transfection refers to the taking up of a vector by a cell whether or not any coding sequences are in fact expressed. Numerous methods of transfection are known to the ordinarily skilled artisan, for example, lipofectamine, calcium phosphate co-precipitation, electroporation, DEAE-dextran treatment, microinjection, viral infection, and other methods known in the art. Transduction refers to entry of a virus into the cell and expression (e.g., transcription and/or translation) of sequences delivered by the viral vector genome. In the case of a recombinant vector, “transduction” generally refers to entry of the recombinant viral vector into the cell and expression of a nucleic acid of interest delivered by the vector genome.

Any of the vectors comprising a nucleic acid sequence that encodes the disclosed polypeptides or components of the present system is also within the scope of the present disclosure. Such a vector may be delivered into host cells by a suitable method. Methods of delivering vectors to cells are well known in the art and may include DNA or RNA electroporation, transfection reagents such as liposomes or nanoparticles to delivery DNA or RNA; delivery of DNA, RNA, or protein by mechanical deformation (see, e.g., Sharei et al. Proc. Natl. Acad. Sci. USA (2013) 110(6): 2082-2087, incorporated herein by reference); or viral transduction. In some embodiments, the vectors are delivered to host cells by viral transduction. Nucleic acids can be delivered as part of a larger construct, such as a plasmid or viral vector, or directly, e.g., by electroporation, lipid vesicles, viral transporters, microinjection, and biolistics (high-speed particle bombardment). Similarly, the construct containing the one or more transgenes can be delivered by any method appropriate for introducing nucleic acids into a cell. In some embodiments, the construct or the nucleic acid encoding the disclosed polypeptides or components of the present system is a DNA molecule. In some embodiments, the nucleic acid encoding the disclosed polypeptides or components of the present system is a DNA vector and may be electroporated to cells. In some embodiments, the nucleic acid encoding the disclosed polypeptides or components of the present system is an RNA molecule, which may be electroporated to cells.

Additionally, delivery vehicles such as nanoparticle- and lipid-based mRNA or protein delivery systems can be used. Further examples of delivery vehicles include lentiviral vectors, ribonucleoprotein (RNP) complexes, lipid-based delivery system, gene gun, hydrodynamic, electroporation or nucleofection microinjection, and biolistics. Various gene delivery methods are discussed in detail by Nayerossadat et al. (Adv Biomed Res. 2012; 1: 27) and Ibraheem et al. (Int J Pharm. 2014 Jan. 1; 459(1-2):70-83), incorporated herein by reference.

### Methods

### a) DNA Synthesis

The disclosure provides methods for guided DNA synthesis and/or amplification. In some embodiments, the methods comprise contacting a defense-associated reverse transcriptase (DRT) with engineered target nucleic acid(s) comprising one or more sequences of interest. The disclosure provided above for the DRT and the engineered target nucleic acid(s) are equally applicable to the present methods.

In some embodiments, the method produces a concatemeric DNA product. Such that the DNA product produced from the one or more sequence(s) of interest in the template region of an engineered target nucleic acid is not only synthesized but amplified to generate more than one copy (e.g., two, three, four, five, six, seven, eight, nine, ten, or more copies) of the DNA product.

In some embodiments, the method produces a DNA product from more than one engineered target nucleic acid. For example, the method may produce a DNA product comprising a sequence corresponding to a template region of a first engineered target nucleic acid and a sequence corresponding to a template region of a second engineered target nucleic acid.

In some embodiments, the DNA product is a single stranded DNA. In some embodiments, the DNA product is a double stranded DNA.

In some embodiments, contacting a defense-associated reverse transcriptase (DRT) with an engineered target nucleic acid(s) comprises introducing the system into the cell. As described above the system may be introduced into eukaryotic or prokaryotic cells by methods known in the art. In some embodiments, the cell is a mammalian cell. In some embodiments, the cell is a human cell.

In some embodiments, introducing the system into the cell comprises administering the system to a subject. In some embodiments, administering comprises in vivo administration. In some embodiments, the administering comprises transplantation of ex vivo treated cells comprising the system.

The disclosed systems and methods for DNA synthesis find use in genome editing, biological research, molecular recording, and nucleic acid sequencing technologies. In some embodiments, the disclosed methods are utilized to provide ssDNA donors, for example, for CRISPR/Cas or homologous directed recombination techniques. The donors can be included in the template region of the engineered nucleic acid as sequences of interest. By producing and amplifying the donors in situ, efficiency of Cas9 and HDR-mediated editing can be improved. In some embodiments, the disclosed methods are utilized to model DNA structural variants. For example, the disclosed systems and methods can be used to generate and model repeat expansions and tandem duplications. Similarly, the disclosed methods can be utilized for protein domain shuffling, e.g., by utilizing two more engineered target nucleic acids each comprising a template region of a single protein domain. In some embodiments, the disclosed methods are utilized for or as a part of sequencing protocols. For example, the disclosed methods find use in spatial transcriptomics—in situ sequencing, duplex-corrected RNA sequencing (e.g., in combination with nanopore sequencing), and the like. In some embodiments, the DNA product can be used as an aptamer for protein-protein interactions. In some embodiments, the disclosed methods are utilized for PCR-free production of DNA libraries. In some embodiments, the DNA product can be a sequestering agent for nucleic acid or protein binding partners. In some embodiments, the DNA product can be used as an immune response stimulating agent.

### b) Identifying Substrates and Products of a Target Defense-Associated Reverse Transcriptase (DRT)

The disclosure provides methods for identifying substrates and products of a target defense-associated reverse transcriptase (DRT). The methods comprise immunoprecipitating the target DRT from a first population of a plurality of cells comprising one or more putative noncoding RNA (ncRNA) templates and the target DRT; isolating co-immunoprecipitated nucleic acids; and sequencing the co-immunoprecipitated nucleic acids.

In some embodiments, the co-immunoprecipitated nucleic acids are DNA. In some embodiments, the co-immunoprecipitated nucleic acids are RNA. In some embodiments, the co-immunoprecipitated nucleic acids are combinations of DNA and RNA.

The immunoprecipitation can be completed using known methods in the art under conditions which do not dissociate the DRT from bound ncRNA templates or products. In some embodiments, the immunoprecipitation makes use of DRTs comprising a tag useful for the immunoprecipitation.

In some embodiments, the methods further comprise introducing into the plurality of cells one or more noncoding putative ncRNA templates and the target DRT, or one or more nucleic acids encoding thereof.

### EXAMPLES

### Materials and Methods

Plasmid and E. coli strain construction: All strains and plasmids used in this study are described in Table 2 and Table 4, respectively. Briefly, plasmids were cloned using a combination of methods, including Gibson assembly, restriction digestion-ligation, ligation of hybridized oligonucleotides, Golden Gate Assembly, and around-the-horn PCR. Plasmids were cloned and propagated in E. coli strain NEB Turbo (sSL0410), and all experiments were performed in E. coli str. K-12 substr. MG1655 (sSL0810). Clones were verified by Sanger sequencing or whole plasmid sequencing. pLG007 (Ec86 retron) and pLG010 (DRT type 2) were gifts from Feng Zhang (Addgene plasmids #157885, #157888).

Phage amplification and plaque assays: Phage T5 (a gift from Michael Laub) was amplified in liquid culture by diluting an overnight culture of MG1655 cells 1:100 in 10 mL fresh LB media, adding 50 μL of phage, and incubating at 37° C. for 3-4 hours. Chloroform was added to a final concentration of 5% to facilitate complete bacterial lysis, after which the lysate was centrifuged at 4,000×g for 10 min to pellet cell debris. The supernatant was passed through a sterile 0.22 μm filter, and the phage-containing filtrate was stored at 4° C.

Small-drop plaque assays were performed as follows: E. coli str. K-12 substr. MG1655 (sSL0810) was transformed with the indicated plasmid construct (see Table 4 for plasmid descriptions and sequences) and plated on solid LB media. Single colonies were inoculated in liquid LB media containing the appropriate antibiotic and grown overnight at 37° C. with shaking. The next day, 100 μL of overnight culture were mixed with 4 mL freshly prepared molten soft agar (0.5% agar in LB media containing the appropriate antibiotic) at 42° C. and poured over solid bottom agar (1.5% agar in LB media containing the appropriate antibiotic) in a 10 cm Petri dish. The soft agar was allowed to solidify for 15 min at RT, during which 10× serial dilutions of phage T5 in LB were prepared. For plating, 3 μL of each phage dilution were spotted onto the surface of the soft agar lawn and were allowed to dry uncovered for 10 min under a laminar flow hood. Plates were incubated at 37° C. for 8-16 hours to allow the formation of plaques. After selecting a phage dilution with clearly distinguishable plaques, plaque forming units (PFU) mL⁻¹ were calculated using the following formula:

$$\frac{1000 \times ( number of plaques )}{3 \times ( phage dilution )} .$$

Phage defense activity was assessed by calculating the fold reduction in efficiency of plating (EOP), which was determined by dividing the PFU mL⁻¹ obtained on a lawn of empty vector (EV) control cells by the PFU mL⁻¹ obtained on a lawn of defense system-expressing cells.

RNA and cDNA immunoprecipitation and sequencing (RIP-seg and cDIP-seq): E. coli str. K-12 substr. MG1655 (sSL0810) was transformed with plasmids encoding C-terminally 3×FLAG-tagged Retron-Ecol or N-terminally 3×FLAG-tagged KpnDRT2 (WT or RT-inactive YCAA mutant), as well as their native flanking sequences (see Table 4 for plasmid sequences). Individual colonies were inoculated in liquid LB with chloramphenicol (25 μg mL⁻¹) and grown at 37° C. to OD₆₀₀ of 0.5. For experiments+/−phage infection, 40 mL cultures were split in half, and phage T5 was added to one half at a multiplicity of infection (MOI) of 5, which was calculated as the ratio of phage PFU to bacterial colony forming units (CFU), assuming 8×10⁸ CFU in 1 mL culture at OD₆₀₀ of 1.0. Uninfected and infected cultures were grown for 1 hr at 37° C. For experiments without phage infection, 20 mL cultures were grown to OD₆₀₀ of 0.5 and directly harvested. Cells were harvested by centrifugation at 4,000×g for 10 min at 4° C., and the supernatant was removed. The pellet was washed with 5 mL of cold TBS (20 mM Tris-HCl, pH 7.5 at 25° C., 150 mM NaCl) and spun down again as before. The supernatant was removed, and the pellet was washed with 1 mL of cold TBS before centrifugation at 10,000×g for 5 min at 4° C. The supernatant was removed, and the pellet was flash-frozen in liquid nitrogen and stored at −80° C.

Antibodies for immunoprecipitation were conjugated to magnetic beads as follows: for each sample, 60 μL Dynabeads Protein G (Thermo Fisher Scientific) were washed 3× in 1 mL IP lysis buffer (20 mM Tris-HCl, pH 7.5 at 25° C., 150 mM KCl, 1 mM MgCl₂, 0.2% Triton X-100), resuspended in 1 mL IP lysis buffer, combined with 20 μL anti-FLAG M2 antibody (Sigma-Aldrich, F3165), and rotated for >3 hours at 4° C. Antibody-bead complexes were washed 2× to remove unconjugated antibodies and resuspended in 60 μL of IP lysis buffer per sample.

Flash-frozen pellets were thawed on ice and resuspended in 1.2 mL IP lysis buffer supplemented with 1× cOmplete Protease Inhibitor Cocktail (Roche) and 0.1 U μL⁻¹ SUPERase·In RNase Inhibitor (Thermo Fisher Scientific). To lyse cells, samples were sonicated using a ⅛″ sonicator probe for 1.5 min total (2 s ON, 5 s OFF) at 20% amplitude. To clear cell debris and insoluble material, lysates were centrifuged at 21,000×g for 15 min at 4° C., and 1 mL supernatant was transferred to a new tube. At this point, two small volumes of each sample (10 μL for RIP-seq and 10 μL for cDIP-seq) were set aside as “input” starting material and stored at −80° C. For immunoprecipitation, each sample was combined with 60 μL antibody-bead complex and rotated overnight at 4° C. The next day, each sample was washed 3× with 1 mL ice-cold IP wash buffer (20 mM Tris-HCl, pH 7.5 at 25° C., 150 mM KCl, 1 mM MgCl₂), using a magnetic rack to immobilize the beads in between each wash. During the final wash, each sample was separated into two separate 500 μL volumes for downstream RIP or cDIP processing.

For RIP elution, the supernatant was removed, and beads were resuspended in 750 μL TRIzol (Thermo Fisher Scientific). After 5 min incubation at RT, the supernatant containing eluted RNA was transferred to a new tube and combined with 150 μL chloroform. Samples were mixed vigorously by inversion, incubated at RT for 3 min, and centrifuged at 12,000×g for 15 min at 4° C. RNA was isolated from the upper aqueous phase using the RNA Clean & Concentrator-5 kit (Zymo Research) and eluted in 15 μL RNase-free water. RNA from input samples was isolated in the same manner using TRIzol and column purification. Purified RNA was stored at −80° C. before proceeding to library preparation.

For cDIP elution, the supernatant was removed, and beads were resuspended in 90 μL IP wash buffer and treated with 5 μg RNase A (Thermo Fisher Scientific) for 30 min at 37° C. Input samples were adjusted to 90 μL with IP wash buffer and treated with RNase A in parallel. SDS was added to IP and input samples to a final concentration of 1%, and samples were treated with 25 μg Proteinase K (Thermo Fisher Scientific) for 30 min at 55° C. Beads were immobilized using a magnetic rack, and the supernatant containing eluted DNA was transferred to a new tube. DNA was isolated using the Monarch PCR and DNA Cleanup kit (NEB), following the Oligonucleotide Cleanup protocol and eluting in 15 μL DNase-free water. For Retron-Ecol samples, DNA was treated with DBR1 (Origene) in reactions containing 2 μL DNA, 0.5 μL DBR1, 1×rCutSmart in 10 pL total volume, in order to cleave the 2′-5′ phosphodiester linkage between msRNA and msDNA. Reactions were cleaned up using the Monarch PCR and DNA Cleanup kit (NEB), with elution in 15 pL DNase-free water. Purified DNA was stored at −80° C. before proceeding to library preparation.

For RIP-seq library preparation (input and RIP eluates), RNA was fragmented by random hydrolysis by combining 7 μL RNA, 6 μL water, and 2 μL NEBuffer 2, and heating to 92° C. for 2 min. To remove DNA and prepare RNA ends for adapter ligation, samples were treated with 2 μL TURBO DNase (Thermo Fisher Scientific) and 2 μL RppH (NEB) in the presence of 1 μL SUPERase·In RNase Inhibitor for 30 min at 37° C. This was followed by treatment with 1 μL T4 PNK (NEB) in 1×T4 DNA ligase buffer (NEB) for 30 min at 37° C. Reactions were column-purified using the Zymo RNA Clean & Concentrator-5 kit and eluted in 10.5 μL RNase-free water. RNA concentrations were quantified using the DeNovix RNA Assay. Illumina sequencing libraries were prepared using the NEBNext Small RNA Library Prep kit, and libraries were sequenced on an Illumina NextSeq 500 in paired-end mode with 150 cycles per end.

For cDIP-seq library preparation, 2 μL of each input sample and 10 μL of each IP eluate were diluted to 15 μL with DNase-free water. Samples were denatured by heating at 95° C. for 2 min, and then immediately placed on ice. Ligation of Illumina adapters and conversion of ssDNA to dsDNA were performed using the xGen ssDNA & Low-Input DNA Library Prep Kit (IDT), and libraries were sequenced on an Illumina NextSeq 500 in paired-end mode with 150 cycles per end.

RIP-seq and total RNA-seq analyses: RIP-seq and corresponding input datasets were processed using cutadapt (v4.2) to remove Illumina adapter sequences, trim low-quality ends from reads, and filter out reads shorter than 15 bp. Reads were mapped to combined reference files containing the MG1655 genome (NC_000913.3) and relevant plasmid sequence, as well as the T5 genome (NC_005859.1) for +/−infection experiments, using bwa-mem2 (v2.2.1) with default parameters. SAMtools (v1.17) was used to sort and index alignments. Coverage tracks were generated using bamCoverage (v3.5.1) with a bin size of 1, separation of top and bottom strand alignments, and scaling of coverage according to sequencing depth (based on the total number of reads passing initial trimming and length filtering). Coverage tracks were visualized in IGV.

For transcriptome-wide analyses of RNAs enriched by RIP-seq, aligned reads were assigned to annotated transcriptome features using featureCounts (v2.0.2) with −s 1 for strandedness. The resulting counts matrices were passed to DESeq2 to calculate fold-change and FDR (using the Benjamini-Hochberg procedure) between input and IP for each annotated transcript. Comparisons were visualized using ggplot2, plotting the “baseMean” (mean normalized counts across all conditions) against log₂(fold change). All comparisons included three independent biological replicates.

For counting of neo repeat junction-spanning reads in RIP input (e.g., total RNA) samples, a custom reference sequence was made which consisted of two concatenated neo cDNA repeats. A 20-bp feature annotation was added, centered at the repeat-repeat junction. Reads were aligned to the custom reference sequence using bwa-mem2, and featureCounts was used to count alignments spanning the junction annotation. The resulting counts were normalized for sequencing depth.

cDIP-seg and total DNA sequencing analyses: Adapter trimming, quality trimming, and length filtering of cDIP-seq and corresponding input datasets were performed as described above for RIP-seq experiments. Trimmed and filtered reads were mapped to combined reference files, sorted, indexed, and plotted onto coverage tracks as described above. Alignments over annotated transcriptome features were counted using featureCounts with −s 2 for strandedness. The resulting counts matrices were processed by DESeq2 and plotted as described above. All transcriptome-wide comparisons were performed using three independent biological replicates.

In order to plot cDNA 5′ and 3′ ends over the KpnDRT2 ncRNA locus, cDIP-seq alignment coordinates were extracted using the bamtobed utility from bedtools (v2.31.0). The 5′ boundary of each read pair was determined as the start coordinate of read 1, for transcripts on the top strand, or the end coordinate of read 1, for transcripts on the bottom strand. Meanwhile, the 3′ boundary of each read pair was determined as the end coordinate of read 2, for transcripts on the top strand, or the start coordinate of read 2, for transcripts on the bottom strand. The boundary coordinates thus defined for each read pair were plotted as a histogram over the KpnDRT2 ncRNA locus.

For counting of reads mapping to the KpnDRT2 cDNA, a custom annotation file was created which defined the DRT2 cDNA feature based on the coverage boundaries from cDIP-seq of KpnDRT2. Alignments over this feature were counted using featureCounts with −s 2 for strandedness and --minOverlap 60. Counting of neo repeat-repeat junction-spanning reads was performed as described above for RIP input samples. The proportion of junction-spanning versus non-junction-spanning cDNA alignments was calculated by dividing the junction-spanning read counts by the total number of reads mapped to the custom concatenated reference sequence.

To analyze cDIP-seq reads with soft-clipped extensions beyond the DRT2 cDNA coverage boundary, cutadapt was used to extract reads containing the full-length KpnDRT2 cDNA and then trim the cDNA repeat sequence from the 5′ end of the read. This step produced trimmed reads containing only the portion of the read extending beyond the coverage boundary. A custom bash script was used to calculate the lengths of the extensions. The extensions were subsequently mapped back to the combined MG1655 genome, T5 phage, and DRT2 plasmid reference using bwa-mem2. Coverage tracks of the alignments were generated using bamCoverage.

dRNA-seq: To precisely map the transcription start site of the KpnDRT2 ncRNA, an RNA-seq library preparation protocol was used to enrich primary transcripts from the total RNA pool, as previously described (C. M. Sharma, J. Vogel, Current Opinion in Microbiology 19, 97-105 (2014)). E. coli MG1655 cells transformed with a plasmid encoding KpnDRT2 were grown to exponential phase, and total RNA was extracted using TRIzol (Thermo Fisher Scientific). 1 μg of total RNA was fragmented in 1×NEBuffer 2 by heating at 92° C. for 1.5 min. DNase treatment was performed with 1 μL TURBO DNase (Thermo Fisher Scientific) in the presence of 1 μL SUPERase·In RNase Inhibitor (Thermo Fisher Scientific) for 10 min at 37° C. Samples were treated with 1 μL T4 PNK in 1×T4 DNA ligase buffer (NEB) at 37° C. for 30 min and purified using the Zymo RNA Clean & Concentrator-5 kit. To enrich primary transcripts with tri-phosphorylated 5′ ends, samples were treated with 1 μL of Terminator Exonuclease (Biosearch Technologies) in 1×Terminator Reaction Buffer A (Biosearch Technologies) supplemented with 0.5 μL SUPERase·In RNase Inhibitor (Thermo Fisher Scientific). Reactions were incubated at 30° C. for 1 hour and stopped by adding EDTA to a final concentration of 5 mM. Samples were purified using the Zymo RNA Clean & Concentrator-5 kit, and then treated with 2 μL RppH (NEB) in 1×NEBuffer 2 supplemented with 1 μL SUPERase·In RNase Inhibitor (Thermo Fisher Scientific). Reactions were incubated at 37° C. for 30 min and purified using the Zymo RNA Clean & Concentrator-5 kit. Illumina sequencing libraries were prepared using the NEBNext Small RNA Library Prep kit, and libraries were sequenced on an Illumina NextSeq 500 in single-end mode with 75 cycles per end.

Adapter trimming, quality trimming, and length filtering of dRNA-seq reads were performed as described above for RIP-seq experiments. Trimmed and filtered reads were mapped to reference files using bowtie2 (v2.4.5) with default parameters. Alignments were sorted and indexed as described above. The locations of RNA 5′ ends over the KpnDRT2 ncRNA locus were determined and plotted as described above for cDNA 5′ end analysis.

Term-seg: Term-seq was performed to enrich the 3′ ends of transcripts, as previously described (D. Dar, et al., Science 352, aad9822 (2016)), using the same RNA sample as used for dRNA-seq. 1 μg of total RNA was treated with 1 μL TURBO DNase in 1×TURBO DNase Buffer (Thermo Fisher Scientific) supplemented with 1 μL SUPERase·In RNase Inhibitor (Thermo Fisher Scientific) for 10 min at 37° C., followed by cleanup using the Zymo RNA Clean & Concentrator-5 kit. Ligation of an i7 Illumina adapter to RNA 3′ ends was performed using the NEBNext Small RNA Library Prep kit, followed by cleanup using the Zymo RNA Clean & Concentrator-5 kit. Samples were fragmented in 1×NEBuffer 2 by heating at 92° C. for 1.5 min, then treated with 2 μL RppH (NEB) in the presence of 1 μL SUPERase·In RNase Inhibitor (Thermo Fisher Scientific) for 30 min at 37° C. This was followed by treatment with 1 μL T4 PNK in 1×T4 DNA ligase buffer (NEB) at 37° C. for 30 min and cleanup using the Zymo RNA Clean & Concentrator-5 kit. Illumina library preparation continued with the remainder of the NEBNext Small RNA Library Prep protocol after the initial i7 adapter ligation step. Libraries were sequenced on an Illumina NextSeq 500 in single-end mode with 75 cycles per end.

Adapter trimming, quality trimming, and length filtering of Term-seq reads were performed as described above for RIP-seq experiments. Trimmed and filtered reads were mapped to reference files using bowtie2 (v2.4.5) with default parameters. Alignments were sorted and indexed as described above. The locations of RNA 3′ ends over the KpnDRT2 ncRNA locus were determined and plotted as described above for cDNA 3′ end analysis.

Long-read DNA sequencing: Total DNA was extracted from E. coli str. K-12 substr. MG1655 (sSL0810) cells transformed with the indicated DRT2 expression vectors, using the Wizard Genomic DNA purification kit (Promega). For experiments performed in the absence of phage infection, single-stranded DNA was converted to double-stranded DNA using the Adaptase and Extension modules of the xGen ssDNA & Low-Input DNA Library Prep Kit (IDT). DNA was then purified using 1.2×AMPure XP beads (Beckman Coulter). This dsDNA conversion step was omitted for experiments performed in the presence of phage, as the Adaptase reaction is biased toward short ssDNA fragments (see user manual), and because phage infection is expected to trigger the in vivo conversion of single-stranded DRT2 cDNA to double-stranded DNA. DNA samples were prepared for long-read sequencing using the Native Barcoding Kit (Oxford Nanopore), following the manufacturer's protocol. Sequencing using an ONT MinION was performed with real time basecalling, barcode balancing, minimum read length of 200 bp, read splitting on, and minimum Q score of 8.

Adapter trimming and barcode trimming were performed with guppy barcoder (v6.5.7). To filter out non-cDNA reads, minimap2 (v2.26) was used to align reads to plasmid reference sequences in which the expected cDNA region had been removed, as well as to the E. coli genome. Unmapped reads were then extracted for downstream analysis using SAMtools. A custom script was used to quantify the number of cDNA repeats detected in each sequencing read, and counts were normalized to the total number of sequenced reads for each sample. For visualization of concatenated cDNAs from the phage-infected KpnDRT2 sample, reads were aligned to an artificial reference sequence using the built-in aligner in Geneious with medium sensitivity and an iteration of up to five times. The artificial reference sequence was created by concatenating up to 50 repeats of the cDNA template. To ensure that reads were aligned to the start of the cDNA concatemer, and not stochastically across the repeated sequence, an ‘anchor’ sequence was appended to the 5′ end of the first strand in all filtered sequences and the beginning of the cDNA concatemer sequence, thereby enforcing synchronous alignment starting at the 5′ end of the cDNA. Coverage over the reference cDNA concatemer was then exported for visualization.

Liquid chromatography with tandem mass spectrometry: E. coli str. K-12 substr. MG1655 (sSL0810) cells transformed with the indicated DRT2 expression vectors were grown at 37° C. in 50 mL LB with chloramphenicol (25 μg mL⁻¹) to OD₆₀₀ of 0.5. Phage T5 was added at MOI 5 and cultures were infected for 1 hour. Cells were harvested by centrifugation at 4,000×g for 10 min at 4° C., and the supernatant was discarded. The pellet was washed with 5 mL cold TBS (20 mM Tris-HCl, pH 7.5 at 25° C., 150 mM NaCl) and spun down again as before. The supernatant was removed, and the pellet was washed with 1 mL of cold TBS before centrifugation at 20,000×g for 5 min at 4° C. The supernatant was removed, and the pellet was flash-frozen in liquid nitrogen and stored at −80° C.

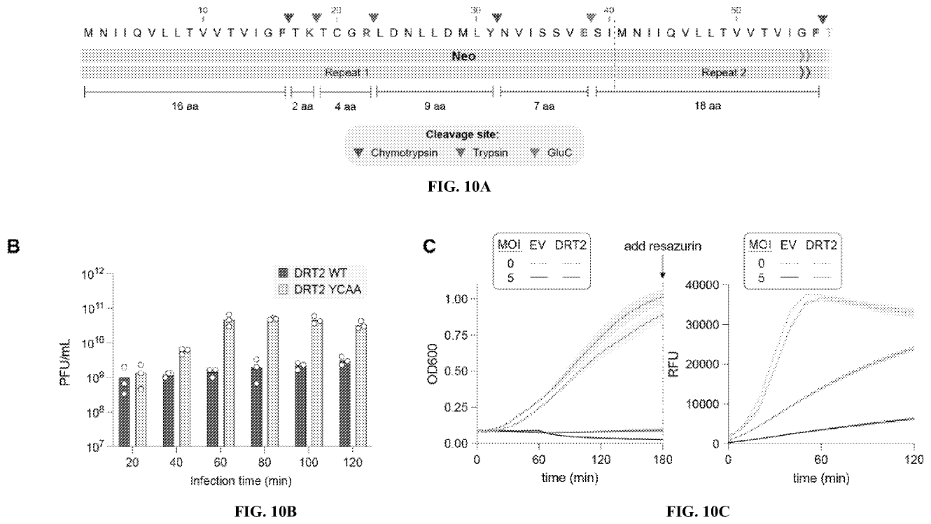

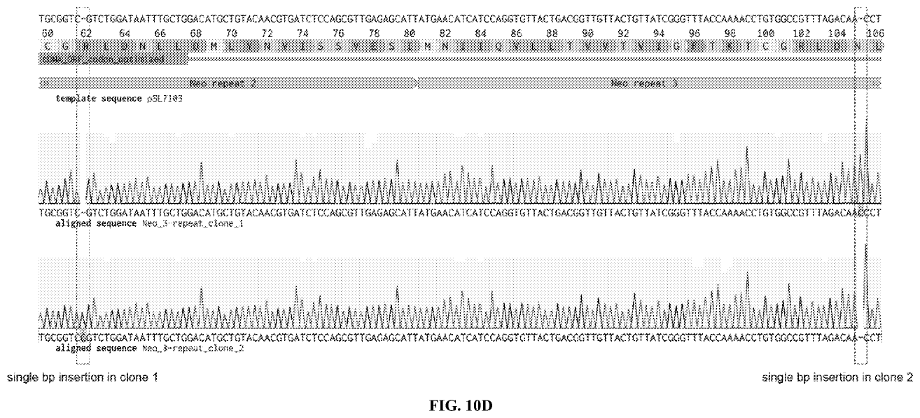

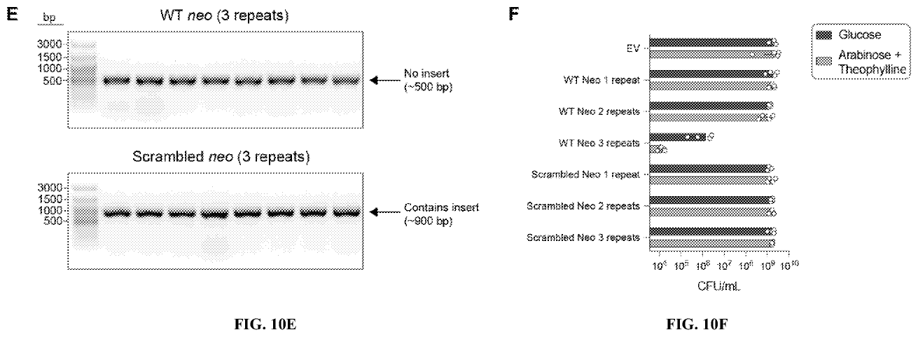

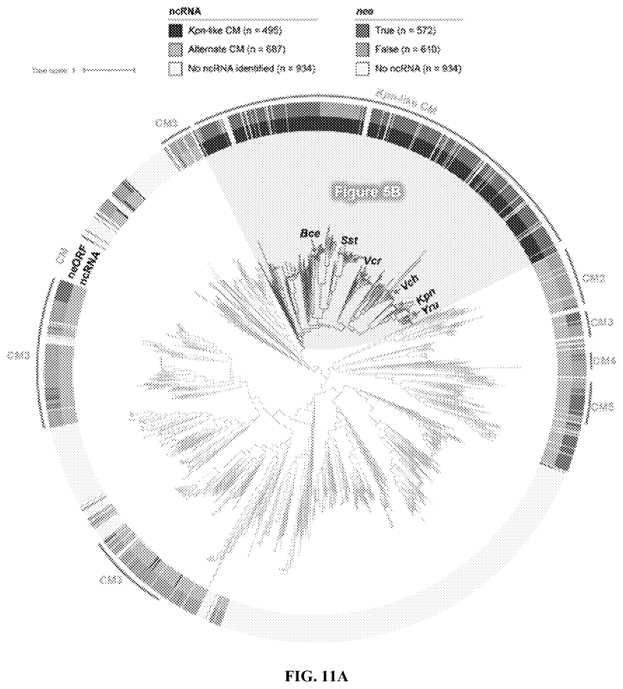

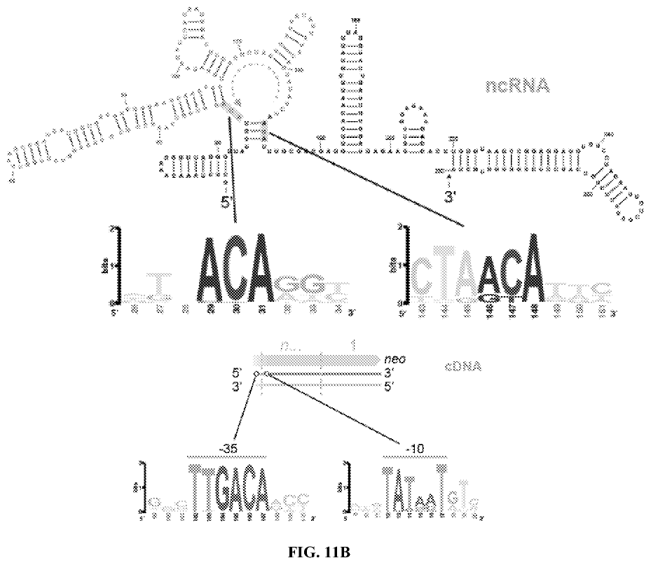

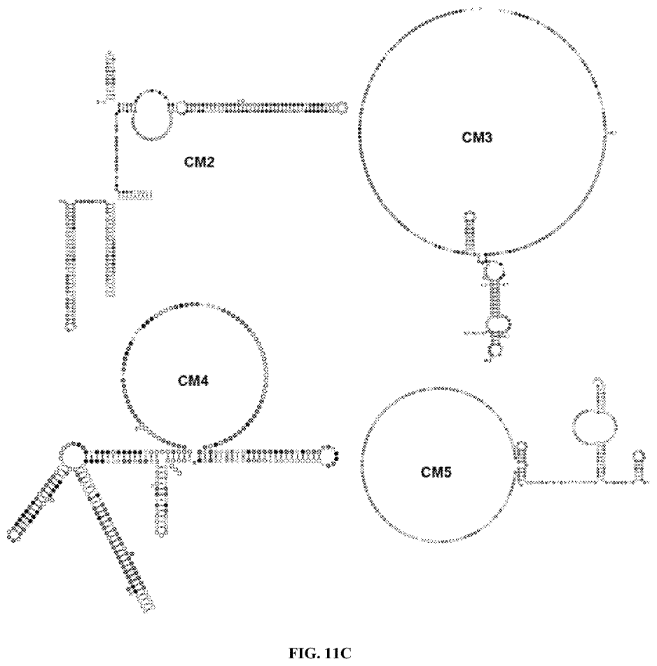

Flash-frozen pellets were thawed on ice and resuspended in 1 mL lysis buffer (100 mM ammonium bicarbonate, 2% sodium deoxycholate). Cells were sonicated using a ⅛″ sonicator probe for 1.5 min total (5 s ON, 10 s OFF) at 20% amplitude. Lysates were heated to 95° C. for 10 min. Protein concentrations were assessed using the Pierce BCA assay (Thermo Fisher Scientific). 50 μg of each sample were subjected to reduction by DTT and alkylation by IAA before being precipitated onto SP3 beads as previously described (C. S. Hughes, et al., Nat Protoc 14, 68-85 (2019)). The beads were washed and then the samples were split in two, to be digested under different digestion conditions. In one condition, proteins underwent on-bead digestion by trypsin, glu-c, and chymotrypsin; this protease mixture was specifically chosen to generate peptides from the Kpn Neo protein in an amino acid length range suitable for detection by LC-MS/MS (FIG. 10A). In the other condition, proteins underwent on-bead digestion by trypsin alone. This more conventional digestion approach was adopted to facilitate the analysis of global proteomic changes that occurred under the different experimental conditions. Each of the proteases was added in a 1:50 enzyme:substrate ratio for overnight digestion at room temperature.

Whole proteome, label-free MS analyses were performed by data-independent acquisition (DIA). Approximately 1 μg of total peptides was analyzed on a Waters M-Class UPLC using a 15 cm IonOpticks Aurora Elite column (75 μm inner diameter; 1.7 μm particle size; heated to 45° C.) coupled to a benchtop Thermo Fisher Scientific Orbitrap Q Exactive HF mass spectrometer. Peptides were separated at a flow rate of 400 nL/min with a 150 min gradient, including sample loading and column equilibration times. Data were acquired in data-independent mode using Xcalibur 4.5 software. MS1 Spectra were measured with a resolution of 120,000, an AGC target of 3×10⁶ and a mass range from 350 to 1600 m/z. Per MS1, 29 equally distanced, sequential segments were triggered at a resolution of 30,000, an AGC target of 3×10⁶, a segment width of 43 m/z, and a fixed first mass of 200 m/z. The stepped collision energies were set to 22.5, 25, and 27.

Two separate searches were conducted for the two digestion conditions. All DIA data were analyzed with Spectronaut software (v18.6) using directDIA analysis methodology against a combined reference database including the E. coli proteome (NCBI RefSeq assembly GCF_000005845.2), T5 phage proteome (NCBI RefSeq assembly GCF_000858785.1), and the KpnDRT2 RT and Neo (5 repeat) sequences. Cysteine carbamidomethylation was set as a fixed modification, and methionine oxidation and N-terminal acetylation were set as variable modifications. For the Neo-targeted experiment, trypsin, glu-c, and chymotrypsin were set as the digestion enzymes. For the global proteomics experiment, trypsin was set as the digestion enzyme. Normalization was performed using ‘automatic normalization’ in Spectronaut. Imputation was performed using ‘global imputation’ in Spectronaut for the global proteomics experiment, and was not performed for the Neo-targeted experiment. For differential protein abundance analysis, calculation of log₂(fold change) and q-value was performed by Spectronaut using three independent biological replicates for each condition.

Infection time course: For time course experiments assessing phage titer and concatenated RNA production during T5 infection of DRT2-expressing cells, E. coli MG1655 cells transformed with plasmids encoding WT or catalytically inactive (YCAA) KpnDRT2 were grown to OD₆₀₀ of 0.4 in 25 ml volume. At the start of the experiment, 1 mL of culture was taken as the t=0 (uninfected) time point for RNA extraction, and then phage lysate was added at MOI of 5. Cultures were incubated with shaking at 37° C. for 2 hours. Every 20 minutes, 200 μL and 1 mL volumes were taken for phage titer measurements and RNA extraction, respectively.

Phage titer measurements: Phage titer measurements were taken over the course of T5 infection by removing 200 μL of culture from the ongoing infection and adding chloroform (5% final concentration) in order to completely lyse cells. Lysates were then centrifuged at 13,000×g for 5 min in order to pellet cell debris. Enumeration of plaque forming units (PFU) was performed using the same plaque assay protocol as described above.

RT-qPCR: Samples for RT-qPCR analysis were prepared with three independent biological replicates and were collected every 20 minutes for 2 hours after infection with T5 at MOI of 5, as described above. At each timepoint, 1 mL volumes of bacterial culture were removed and centrifuged at 3,000×g for 3 min. The supernatant was removed, and the resulting pellet was resuspended in 750 μL of TRIzol and incubated at room temperature for 5 min. 150 μl of chloroform were added, and samples were mixed by shaking and centrifuged at 12,000×g for 15 min at 4° C. The upper aqueous phase was transferred to a new tube and mixed with an equal volume of absolute ethanol. Total RNA was purified using the Monarch RNA Cleanup Kit (NEB) and stored at −80° C.

cDNA synthesis was performed using 500 ng of total RNA as the input, which was first treated with 1 μl of dsDNase (Thermo Fisher Scientific) in 1×dsDNase reaction buffer in a final volume of 10 μL, and incubated at 37° C. for 2 min. Reactions were stopped by adding DTT to a final concentration of 10 mM and heating to 55° C. for 5 min. Reverse transcription was performed using the iScript cDNA Synthesis Kit (BioRad) following the manufacturer's instructions. The samples were stored at −20° C.

Quantitative PCR was performed in 10 μL reactions containing 5 μL SsoAdvanced Universal SYBR Green Supermix (BioRad), 0.5 μL of each primer pair at 10 μM concentration, and 4 μL of 25-fold diluted cDNA. Primers were designed to span the cDNA repeat junction. For normalization, primer pairs that anneal to the reference gene rrsA were used. Reactions were prepared in 384-well PCR plates (BioRad), and measurements were performed on a CFX384 RealTime PCR Detection System (BioRad) using the following thermal cycling parameters: polymerase activation and DNA denaturation (98° C. for 2.5 min), 40 cycles of amplification (98° C. for 10 s, 62° C. for 20 s), and terminal melt-curve analysis (decrease from 95° C. to 65° C. in 0.5° C./5 s increments). Values are plotted as abundance of concatenated RNA, relative to rrsA, relative to the WT sample at t=0 (2^{-ΔΔCq}).

Northern blotting: RNA samples collected for RT-qPCR analysis, described above, were also used for Northern blotting analysis. After RNA purification by TRIzol and the Monarch RNA Cleanup Kit, samples were treated with TURBO DNase in TURBO DNase buffer (Thermo Fisher Scientific) for 30 min at 37° C. Reactions were cleaned up using the Monarch RNA Cleanup Kit, and RNA concentrations were measured using the DeNovix RNA Assay.

Northern blotting was performed as previously described (K. M. McKenney, et al., RNA, rna.079880.123 (2024)), with modifications. In brief, equal amounts of RNA (1.2 μg) were adjusted to 8 μL total volume with water and combined with 22 μL denaturing mix (15 μL formamide, 5.5 μL formaldehyde, and 1.5 μL 10×MOPS). Samples were heated at 55° C. for 15 min prior to separation on a denaturing agarose gel (1% agarose, 3.7% formaldehyde, 1×MOPS buffer) for 2.5 hours at 80 V. RNA was transferred to a Hybond-N+ membrane (GE Healthcare) by upward capillary transfer in 10×SSC (1.5 M NaCl, 0.15 M trisodium citrate dihydrate, pH 7). The next day, RNA was crosslinked to the membrane using a UV crosslinker, and the membrane was pre-hybridized in ULTRAhyb-Oligo buffer (Thermo Fisher Scientific) for 1 hour at 42° C. A biotinylated oligonucleotide probe specific for the concatenated RNA repeat-repeat junction was added to the hybridization buffer at a final concentration of 5 nM and hybridization was performed overnight at 42° C. The next day, the membrane was washed twice with Wash Buffer 1 (2×SSC with 0.1% SDS) and twice with Wash Buffer 2 (0.1×SSC with 0.1% SDS). The membrane was developed using the Chemiluminescent Nucleic Acid Detection Module Kit (Thermo Fisher Scientific) and imaged with an Amersham Imager 600 (GE Healthcare). The membrane was then stripped using boiling 0.1% SDS, pre-hybridized with ULTRAhyb-Oligo buffer, and reprobed using a biotinylated oligonucleotide probe specific for 16S rRNA. Hybridization, washes, and imaging were done as before.

Infection response growth curves: Overnight cultures of E. coli MG1655 cells transformed with either empty vector (EV) or WT DRT2 expression vector were diluted 1:100 in LB with chloramphenicol (25 μg mL⁻¹), grown to exponential phase, and normalized to OD₆₀₀ of 0.2. 180 μL of cell culture were transferred into wells of a 96-well optical plate containing 20 μL of T5 lysate diluted to result in a final MOI of 5 or 0.05, or 20 μL of LB for the uninfected condition. The plate was incubated for 5 hours at 37° C. with shaking. OD₆₀₀ values were recorded every 10 minutes using a Synergy Neo2 microplate reader (Biotek).

Resazurin cell viability assays: Cell viability was evaluated with the resazurin-based reagent alamarBlue HS (Thermo Fisher Scientific). 200 μL cultures were prepared as described above for cell growth experiments performed at varying MOIs. Infections proceeded for 3 hours before 180 μL of cell culture were mixed with 20 μL of alamarBlue HS and incubated with shaking at 37° C. During incubation, fluorescence was measured in red fluorescence units every 10 min according to the manufacturer's guidelines, using a Synergy Neo2 microplate reader (Biotek) with a monochromator module set to a fixed gain setting of 75. The fluorescence of blank LB controls was subtracted as background from all other measured values.

Neo induction experiments: Cellular growth curves: E. coli MG1655 cells were transformed with plasmids encoding various repeat lengths of WT or mutant Neo. Individual colonies were inoculated in LB supplemented with kanamycin (50 μg mL⁻¹) and glucose (2%) and grown until cells reached OD₆₀₀ 0.8-1.0. The cells were then pelleted and resuspended in LB media with kanamycin (50 μg mL⁻¹). For each sample, OD₆₀₀ density was normalized to 0.1, and 200 μL of cell suspension were transferred to a 96-well clear-bottom plate. The OD₆₀₀ was measured using a Synergy Neo2 microplate reader (Biotek) while shaking at 37° C. for 50 min (until OD₆₀₀ reached ~0.3). Neo expression was then induced by the addition of arabinose (final concentration 0.5%) and theophylline (final concentration 0.5 mM), and cell growth was monitored for another 2 hours. For experiments testing the induction of diverse Neo homologs, growth rates were calculated using the formula

$$μ = \frac{ln ( OD {600}_{t} ) - ln ( OD {600}_{0} )}{t - {t}_{0}} .$$

The time window of 30 minutes to 80 minutes after induction was used to calculate the growth rate for each condition.

Spot assays and CFU counting after Neo induction: To assess cell viability after KpnNeo induction, a small volume from each well of the growth curve experiment described above was taken for plating on LB agar. 10× serial dilutions of each culture were prepared and spot-plated on LB agar supplemented with either kanamycin (50 μg mL⁻¹) and glucose (2%), or kanamycin (50 μg mL⁻¹), arabinose (0.5%), and theophylline (0.5 mM). Plates were incubated overnight at 37° C., and colony forming units per milliliter (CFU/mL) were counted the next day.

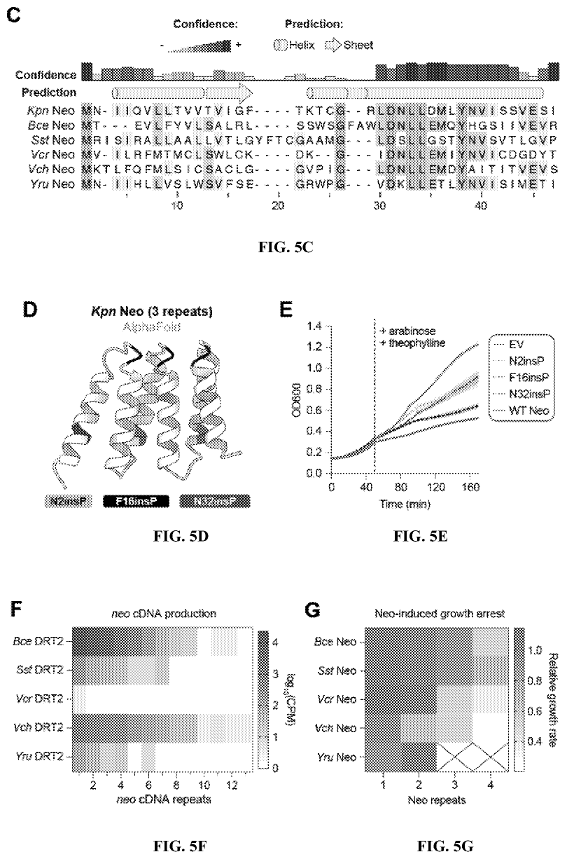

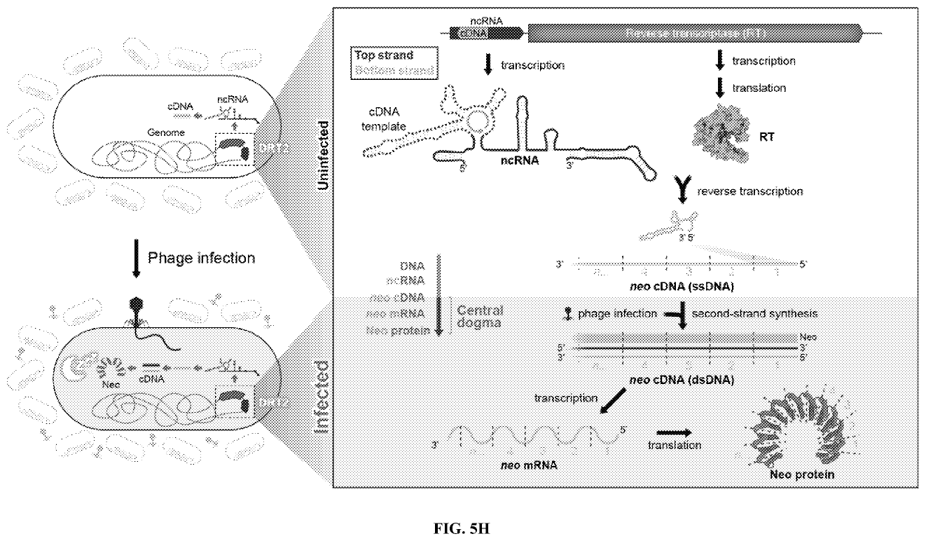

Protein secondary structure prediction: Six Neo protein sequences were aligned with MAFFT (LINSI option; v7.520), and the resulting alignment was visualized with Jalview (v2.11.3.2). Secondary structural elements were predicted by submitting the alignment to Ali2D. The consensus predicted structure annotations and mean confidence values are plotted above the alignment in FIG. 5C.

Protein tertiary structure prediction: The Neo 3D structure was modeled using three independent prediction tools. The primary amino acid sequence of KpnNeo was used as input for AlphaFold2 using MMseqs2 (via ColabFold), and the same sequence was used as input for ESMFold. A multiple sequence alignment (MSA) of the Neo homologs shown in FIG. 5C was used as input for trRosetta. All predictions were based on 3 concatenated repeats of Neo.

Start codon prediction: The Kpn neo start codon was predicted using the RBS Calculator tool, using one cDNA repeat unit as the input sequence and specifying K. pneumoniae as the host organism.

Sequence identity matrices: Pairwise sequence identity matrices were generated in Geneious from MAFFT alignments of ncRNA and cDNA nucleic acid sequences, or of RT and Neo amino acid sequences, using default settings. RT proteins are listed in Tables 1 and 5.

RNA secondary structure prediction: KpnDRT2 ncRNA secondary structure prediction (from single-sequence input) was performed using RNAfold and visualized using RNAcanvas.

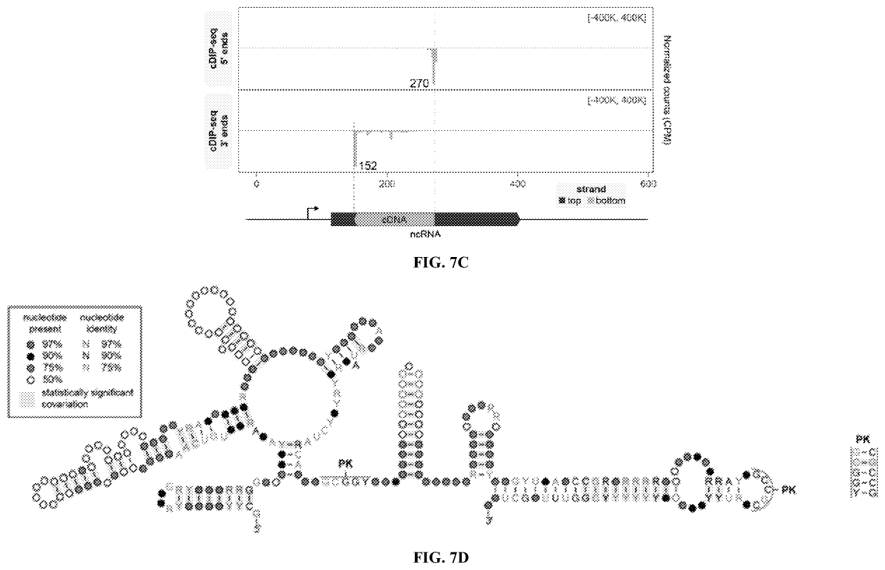

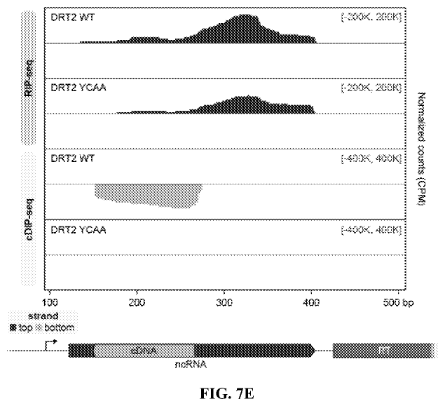

ncRNA covariance modeling: Homologs of KpnDRT2 were identified using the RT amino acid sequence (WP_012737279.1) as the seed query in a BLASTP search of the NR protein database (max target sequences=100). Nucleotide sequences 1 kb upstream and downstream of RT genes were retrieved, clustered at 99.9% sequence identity to remove replicates using CD-HIT (v4.8.1), and aligned using MAFFT (v7.505). The resulting alignment was trimmed at the 5′ and 3′ ends to the exact boundaries of the ncRNA, as determined by RIP-seq experiments with KpnDRT2. These putative ncRNA sequences were clustered at 95% sequence identity using CD-HIT and realigned using mLocARNA (v1.9.1) with default parameters. The resulting structure-based multiple sequence alignment was used to build and calibrate a covariance model (CM) using the Infernal suite (v1.1.4). The CMsearch function of Infernal was then used to scan through nucleotide sequences of additional drt2 loci and 1-kb flanking regions, generated by an expanded BLASTP search (max target sequences=5000) queried on KpnDRT2 and clustered at 85% sequence identity using CD-HIT. The final hits (n=303 DRT2 loci, including KpnDRT2) from the CM used to identify KpnDRT2-like ncRNAs were evaluated for statistically significant co-varying base pairs with R-scape at an E-value threshold of 0.05 (FIG. 7D).

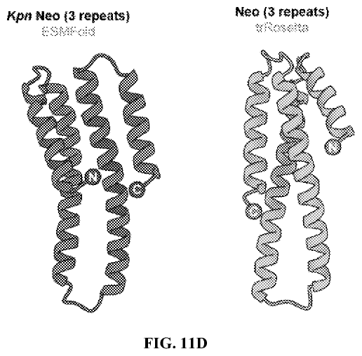

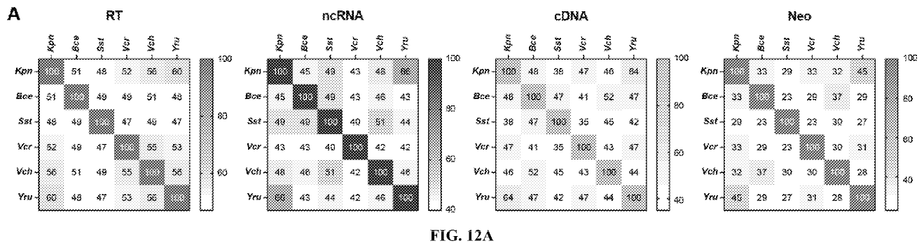

Phylogenetic analyses: An initial set of DRT2 sequences was identified by querying the KpnRT protein sequence (WP_012737279.1) against the NR database with PSI-BLAST (3 iterations; default settings). The top 500 results from this search did not produce any clusters at a threshold of 80% amino acid identity, so this diverse set of homologs was used for an additional BLASTP (−evalue 0.01-max_target_seqs 1000000) search of a local copy of the NCBI NR database (downloaded on Apr. 4, 2023). The resulting hits were further restricted to an e-value cutoff of 1×10⁻³⁰, resulting in a set of 3,056 protein accessions, for which identical protein group (IPG) information was pulled from NCBI with the Batch Entrez tool. Where possible, two genomes encoding each unique DRT2 homolog were randomly sampled from the IPG information, and these genomic sequences were retrieved from NCBI with the Batch Entrez. DRT2 homologs for which IPG information or genomic sequences were unable to be retrieved were removed from the analysis, resulting in a final dataset of 2,116 DRT2 homologs (Table 5). 616 protein sequences in this final DRT2 dataset were also identified as DRT2 homologs in a previous analysis of reverse transcriptases, while no other non-DRT2 homologs from that previous analysis were present in the dataset. Finally, this set of DRT2 sequences was aligned with MAFFT (LINSI option; v7.520) and a phylogenetic tree was constructed with FastTree (-wag -gamma; 2.1.11), before being visualized with iTOL. A subtree of KpnDRT2-like sequences was constructed by manually subsetting the tree in FIG. 11A to include only a monophyletic clade encompassing KpnDRT2. Eight sequences with unexpectedly long branch lengths were manually pruned from this subtree, resulting in a phylogeny of 539 KpnDRT2-like sequences (FIG. 5B).

Systematic ncRNA and Neo prediction: An updated KpnDRT2-like CM (v2) was built by retrieving genomes for the top 500 DRT2 hits from the PSI-BLAST search described above, extracting the DRT2 loci (drt2+/−1 kb), searching each locus with the CMsearch function of Infernal (v1.1.5; default parameters), aligning hits that met the inclusion threshold (n=287) with LocARNA (v2.0.0; mlocarna option with default parameters), and building a CM with the CMbuild function of Infernal (v1.1.5; default parameters).

DRT2 loci, corresponding to the RT gene and 1 kb of upstream and downstream sequence, were then extracted from genomes encoding the 2,116 DRT2 homologs described above, using coordinates in the IPG dataset. To identify putative ncRNA sequences, these loci were queried with the KpnDRT2-like ncRNA CM (v2) using the CMsearch function of Infernal (v1.1.5), with default parameters. Hits that met the inclusion threshold (e-value<0.01) were extracted using the coordinates in the CMsearch output, and these putative ncRNA sequences were de-duplicated, prior to alignment of the sequences with the DECIPHER package (v2.30.0) in R. The KpnDRT2 locus was used as a reference to extract likely cDNA regions from the resulting alignment.

To predict Neo sequences in homologous DRT2 loci, the reverse complements of putative cDNA sequences were assessed in all three possible reading frames to determine which frame contained the fewest stop codons; these were assumed to be the neo open reading frames (ORFs). Start codons (ATG, GTG, TTG) were then probed in the resulting putative neo ORFs, and the start codon of neo was presumed to occur after the first ten amino acids of the putative reading frame translation, consistent with the KpnDRT2 locus. Putative Neo sequences were then constructed by concatenating the translation of this downstream start codon in the putative neo ORF through the end of the putative cDNA (which represents the first unit of cDNA produced by the putative DRT2 rolling circle mechanism; repeat 1), to a translation of the full length putative neo ORF (which represents cDNA units produced by successive rounds of the DRT2 rolling circle mechanism; repeat 1+n). Putative neo sequences that did not contain an internal stop codon, were then checked to determine if the final ten amino acids of the Neo sequence (e.g., translated from repeat 1+n) were identical to the final ten amino acids of Neo translated from the first unit of cDNA synthesis (e.g., translated from repeat 1) (FIG. 5A). Putative Neo sequences that met this criterion were predicted to represent bona fide Neo protein products of DRT2 immune systems.

Putative ncRNA sequences identified with the KpnDRT2-like ncRNA CM (v2) were primarily restricted to a monophyletic clade that included KpnDRT2 (FIG. 5B). An alignment of these ncRNA sequences was built with the DECIPHER package in R, and sequence logos (FIG. 111B) were generated with the web-based version of WebLogo. Logos of cDNA sequences with identified Neo proteins (FIG. 111B) were similarly built from an MSA generated with DECIPHER. To identify ncRNAs in other regions of the larger phylogenetic tree presented in FIG. 11A, additional CMs were constructed via the same approach described above (e.g., mlocarna with default settings, CMbuild in Infernal) by manually selecting regions of the tree that had CM hits for at least three closely related DRT2 sequences. These CMs were used to search the DRT2 loci, and then new CMs were built from the resulting hits; this process was iterated until ncRNAs had been identified across most DRT2 systems. Exemplary ncRNA CMs generated in this process are shown in FIG. 11C. Finally, Neo sequences were predicted in these newly identified putative ncRNAs using the same approach described above.

### Example 1

### cDNA Synthesis by a Defense-Associated Reverse Transcriptase

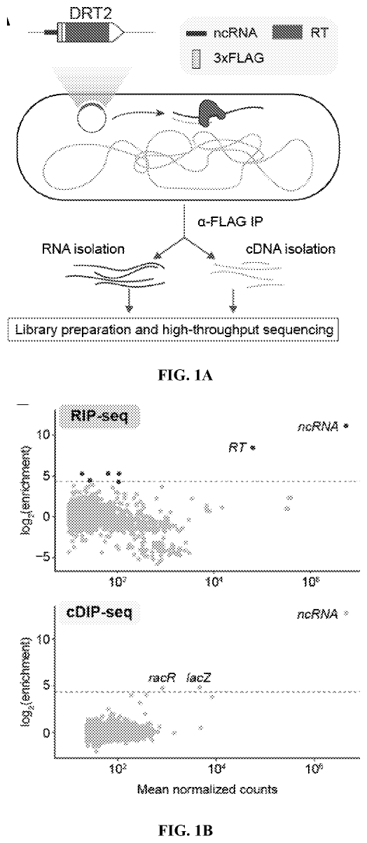

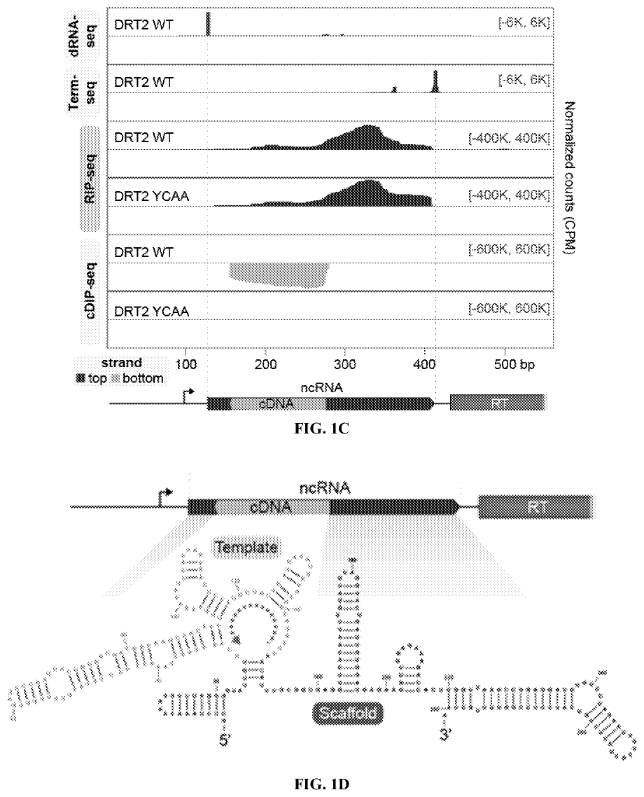

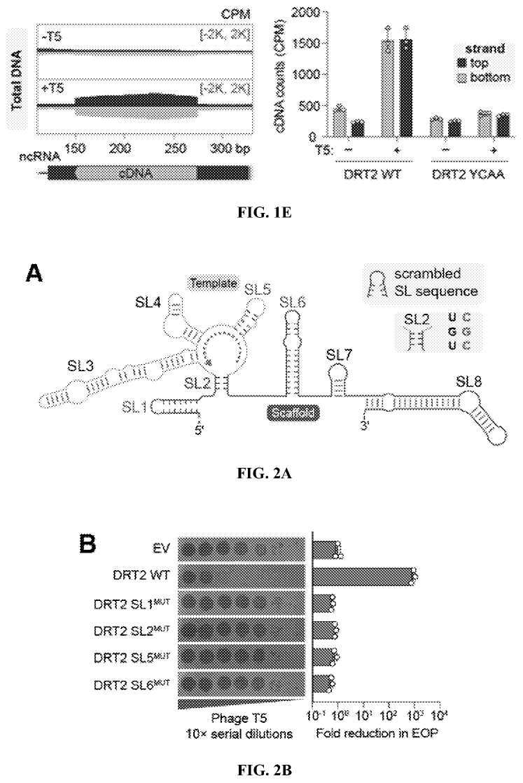

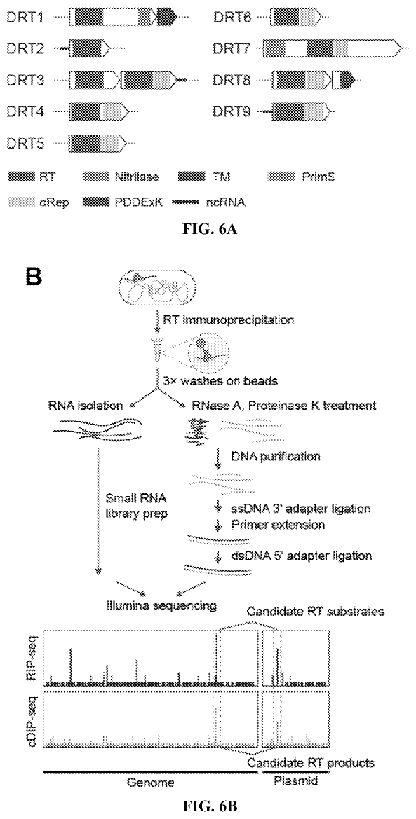

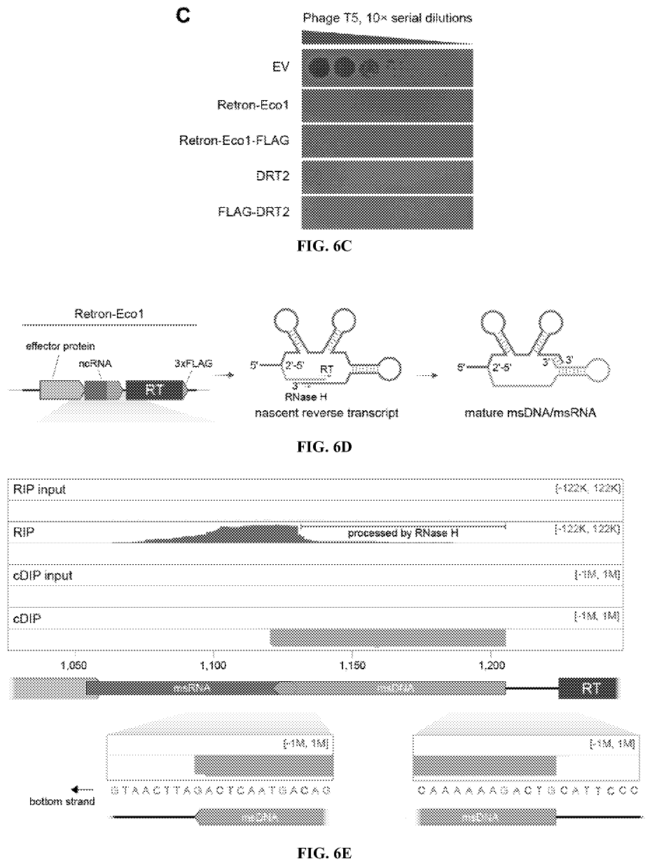

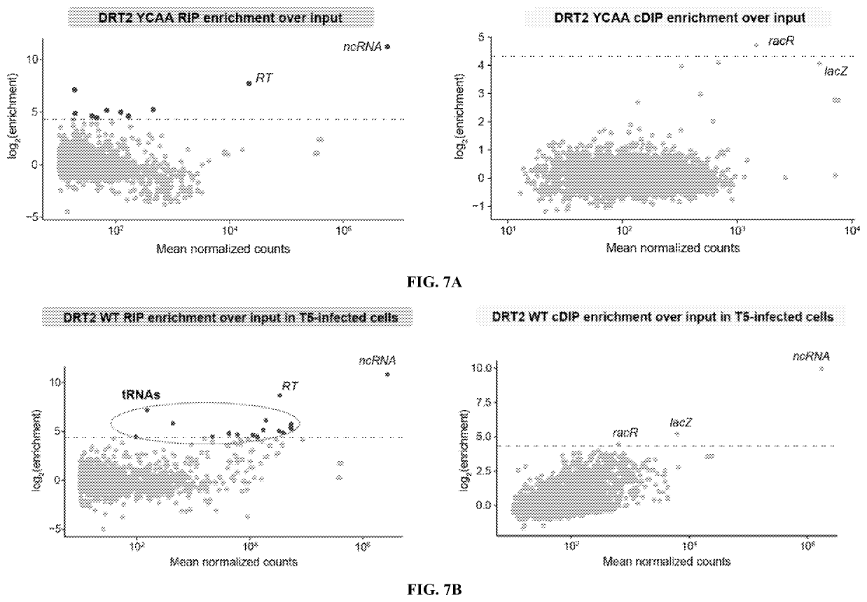

Unlike most other DRT and retron systems, which typically encode a reverse transcriptase enzyme alongside one or more additional protein domains predicted to function as effectors of the immune response, DRT2 systems lack additional protein-coding genes, and the RT protein lacks domains beyond the predicted RNA-directed DNA polymerase (FIG. 6A). To identify the cDNA product of the RT enzyme, a sequencing approach was developed to systematically identify RT-associated cDNA synthesis products based on immunoprecipitation of FLAG-RT fusions, herein referred to as cDNA immunoprecipitation and sequencing (cDIP-seq), performed alongside traditional RNA immunoprecipitation (RIP)-seq to also capture RT-associated RNA substrates (FIGS. 1A and 6B). This approach was validated on the Retron-Ecol (formerly Ec86) after first verifying that the FLAG-tagged retron RT retained phage defense activity comparable to the WT RT (FIG. 6C). RIP-seq and cDIP-seq of Retron-Ecol recapitulated all known features of both the msRNA and msDNA, including RNase H processing of msRNA, and precise 5′ and 3′ ends of the msDNA (FIGS. 6D and 6E). These results increased confidence that a similar approach could provide new insights into the DRT2 molecular mechanism, and these same methods were used with a candidate system from Klebsiella pneumoniae (KpnDRT2).

After confirming that fusing the KpnDRT2 RT with a FLAG epitope tag did not affect defense activity against T5 phage (FIG. 6C), RIP-seq and cDIP-seq were performed with cells constitutively expressing plasmid-encoded ncRNA and RT from their native promoter, followed by genome-wide analyses to identify RNA and cDNA molecules enriched by IP. The resulting datasets revealed that the highest enriched RNA and cDNA transcripts mapped to the KpnDRT2 ncRNA locus (FIG. 1i), suggesting that the primary substrate for reverse transcription by DRT2 is encoded in cis, similar to retron systems. Other enriched hits from cDIP-seq data were also found in control experiments using a catalytically inactive RT mutant (hereafter YCAA), suggesting a spurious origin (FIG. 7A). Interestingly, RIP-seq and cDIP-seq experiments in the presence of T5 phage also revealed a strong and specific enrichment of transcripts derived from the DRT2 ncRNA locus (FIG. 7B), indicating that RT substrate choice is largely unchanged during phage infection.

Mapping of RIP-seq and cDIP-seq data onto the KpnDRT2 locus revealed the presence of a large ncRNA and a seemingly well-defined cDNA with the opposite strandedness relative to the ncRNA, as expected for reverse transcription (FIG. 1C). Control experiments with the inactive YCAA RT mutant demonstrated that ncRNA enrichment occurred independently of reverse transcriptase activity, whereas cDNA enrichment from this locus required an intact RT active site (FIGS. 1C and 7A). RNA-seq library preparation protocols based on dRNA-seq and Term-seq were leveraged to demarcate the precise 5′ and 3′ ends of the ncRNA (FIG. 1C), while analyzing start and end coordinates from cDIP-seq alignments to define the 5′ and 3′ ends of the cDNA (FIG. 7C). Interestingly, dRNA-seq data revealed a single transcription start site (TSS) upstream of the ncRNA, but not the RT gene (FIG. 1C), suggesting that the ncRNA and RT share an upstream promoter, and are separated into mature transcripts via an unknown processing step. A multiple sequence alignment (MSA) of DRT2 homologs was used to generate a covariance model of the ncRNA (FIG. 7D), which was in excellent agreement with the KpnDRT2 ncRNA secondary structure predicted by in silico RNA folding (FIG. 1D). The ncRNA featured numerous conserved stem-loop (SL) elements, a template region corresponding to the cDNA product abutted by a short basal stem, and a large 3′ region that could serves as a scaffold for sequence and/or structure-guided recruitment of the RT.

cDIP-seq data largely recapitulated the same observations made in the absence of phage (FIGS. 7B and 7E). Total DNA sequencing using the input controls from cDIP-seq experiments revealed a strong induction in KpnDRT2 cDNA levels upon phage infection (FIG. 1E). Surprisingly, while cDNA synthesis products in the absence of phage were predominantly single-stranded, with opposite strandedness to the ncRNA, the presence of phage induced higher levels of both the initial cDNA product and its reverse complement (FIG. 1E). The RT may possess both RNA-templated and DNA-templated DNA polymerase activity and that conversion of ssDNA to dsDNA may be a key step within the antiviral defense pathway.

### Example 2

### Rolling-Circle Reverse Transcription Generates Concatenated cDNA Products

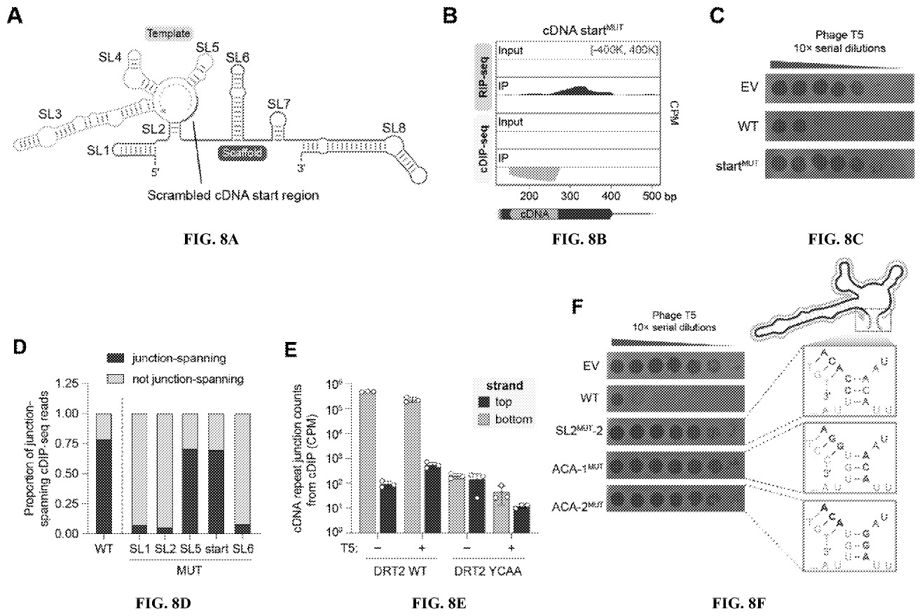

The sequence of individual SLs throughout the ncRNA were mutated in order to eliminate base-pairing, focusing on SL1 at the 5′ end, SL2 at the base of the template region, SL5 within the template region, and SL6 within the scaffold region (FIG. 2A). Mutations to all four regions led to a complete loss of phage defense activity, indicating possible defects in ncRNA binding, cDNA synthesis, or both (FIG. 2B). When ncRNA binding and cDNA synthesis were directly interrogated by the RT using RIP-seq and cDIP-seq, respectively, SL1 and SL6 mutants led to either a partial or complete loss of cDNA synthesis, likely due to disruptions in the positioning of the RT on the ncRNA (FIG. 2C). The SL5 mutant exhibited strong ncRNA and cDNA enrichment, as did an additional mutant in which the region surrounding the cDNA synthesis start site was scrambled (FIGS. 8A-8C), defense activity depends on not only cDNA synthesis, but also on the sequence of the cDNA product itself. The sequence of the template region was completely unchanged and cDNA production resembled the WT system, and yet phage defense was completely abolished (FIGS. 2B and 2C). Beyond production of cDNA with the appropriate ncRNA-specified sequence, additional features of the cDNA product underlie phage defense activity.

Following inspection of the 3′ termini of cDIP-seq reads more closely, it was found that the large majority of reads extended well beyond the boundary defined by the coverage signal; these extensions had been soft-clipped from the reads by conventional alignment algorithms (FIG. 2D). To determine the identity of these soft-clipped extensions, their sequences were extracted and mapped back to the plasmid and E. coli genome. Remarkably, these sequences derived from the 5′ end of the cDNA (FIG. 2D), suggesting a template jumping mechanism whereby the RT proceeds from the end of the template region back to the start, resulting in concatenated cDNA repeat products. Whereas the concatenated cDNAs generated by the WT system had a precise and uniform head-to-tail junction, including one additional nucleotide immediately adjacent to SL2, junction sequences for the SL2 mutant were more heterogeneous (FIG. 2D). Indeed, when the frequency of the expected junction sequence across all tested ncRNA mutants was quantified, only the SL5 and cDNA start mutants retained WT levels of the repeat junction, whereas all other SL mutants nearly eliminated the expected template jumping products (FIG. 8D).

The concatenated cDNA products in total DNA samples from cells +/−T5 phage infection were quantified. T5 phage infection triggered a large increase in the abundance of bottom-strand junction-spanning reads, corresponding to the initial products of RNA-templated DNA synthesis (FIG. 2E). This was matched with a concomitant increase in top-strand junction-spanning reads (FIG. 2E), suggesting that concatenated cDNA synthesis products are efficiently converted into dsDNA in a phage and RT-dependent manner. Similar analyses from cDIP-seq datasets showed a lesser decrease in top-strand junction-spanning reads during phage infection (FIG. 8E), which may be due to RT having lower affinity for the dsDNA generated by second-strand cDNA synthesis, such that it releases these products in cells and/or during immunoprecipitation.

Long-read Nanopore sequencing was leveraged to assess the length of concatenated cDNA products, and to obtain orthogonal evidence of template jumping with a PCR-free approach. Remarkably, KpnDRT2 cDNA products from phage-infected cells spanned a dramatic range of repeat lengths from 1-40 (FIG. 2F), revealing that reverse transcription by KpnDRT2 is highly processive and involves many consecutive rounds of template jumping to generate long concatenated cDNA (from 120 to ~5000 bp). Finally, careful inspection of the sequence and secondary structure of the ncRNA was completed to better understand the mechanism of template jumping. Concatenation of cDNA repeats occurs between the sequences directly abutting SL2, and the terminal 3-nt of each repeat are templated by a conserved 3′-ACA-5′ (ACA-1) whose sequence perfectly matches the right half of SL2 (ACA-2; FIG. 2G). The RT may dynamically melt SL2 during each round of reverse transcription, allowing the terminal 5′-TGT-3′ of nascent cDNA transcripts to equilibrate between hybridization to ACA-1 and ACA-2, and thus prime a subsequent round of cDNA synthesis (FIG. 2G). This model was supported by a complete loss of defense activity in ncRNA mutants disrupting homology between the ACA motifs (FIG. 8F). The proposed cDNA concatenation mechanism resembles rolling-circle DNA replication, and henceforth refers to the generation of concatenated cDNA products as rolling-circle reverse transcription (RCRT; FIG. 2G).

### Example 3

### Concatenated cDNAs Encode a Translated Open Reading Frame (ORF)

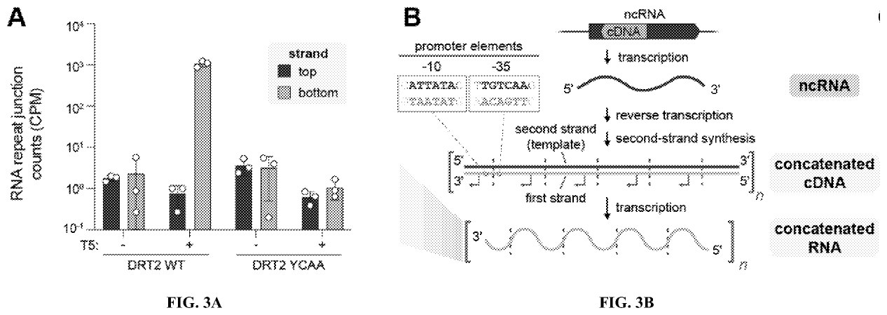

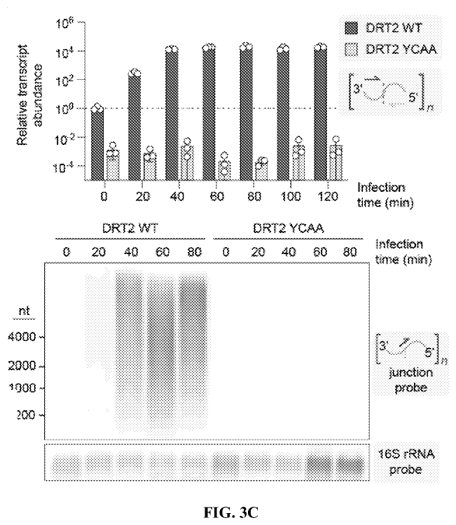

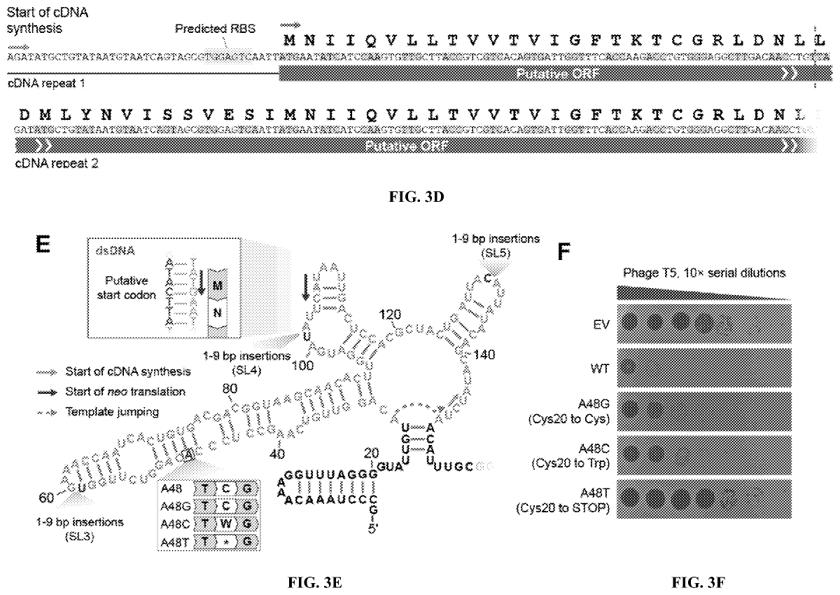

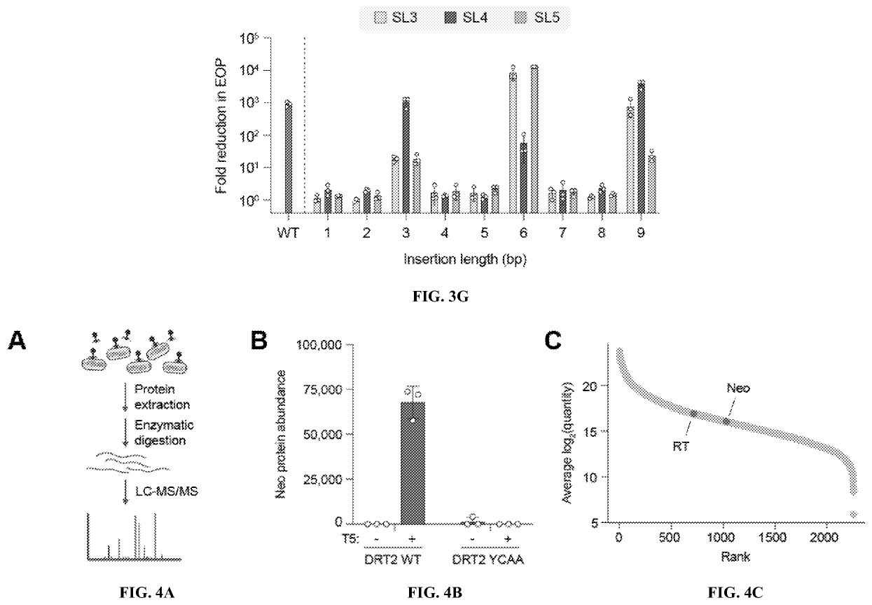

The RIP-seq input controls, which represent total RNA-seq datasets, were re-analyzed for the presence of reads spanning the repeat junction. Such reads were abundantly detected in KpnDRT2 samples, but they depended on the presence of phage and an active RT, and their strandedness was opposite to that of the ncRNA (FIG. 3A). cDNA second-strand synthesis might generate a template strand for another round of transcription by RNA polymerase (FIG. 3B). The resulting transcript would have opposite strandedness to the initial ncRNA and would contain multiple repeats of the cDNA sequence.

In agreement with this, inspection of the cDNA sequence produced by RCRT revealed consensus promoter elements spanning the repeat junction (FIG. 3B), highly reminiscent of transposon promoters that are selectively formed upon DNA circularization during the transposon excision step. Phage-induced second-strand cDNA synthesis might serve to trigger the production of a high-copy concatenated RNA molecule with downstream antiphage function. Consistent with this, concatenated RNAs were strongly induced shortly after phage infection by ~10,000-fold, in a time-course infection experiment (FIG. 3C). Northern blot analysis from phage-infected cells using a probe selective for the chimeric junction revealed a broad size distribution spanning hundreds to thousands of nucleotides (FIG. 3C), in agreement with the large size of cDNA products observed via Nanopore sequencing (FIG. 2F).

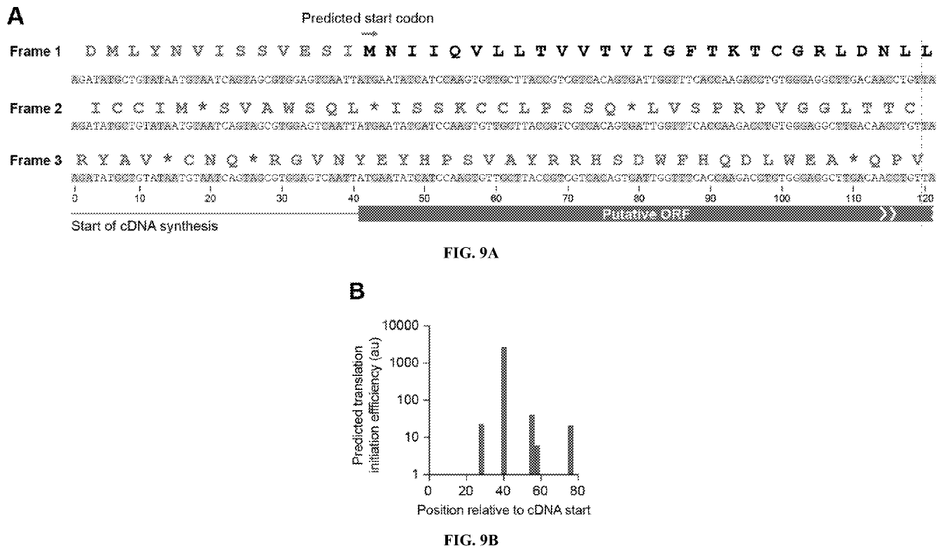

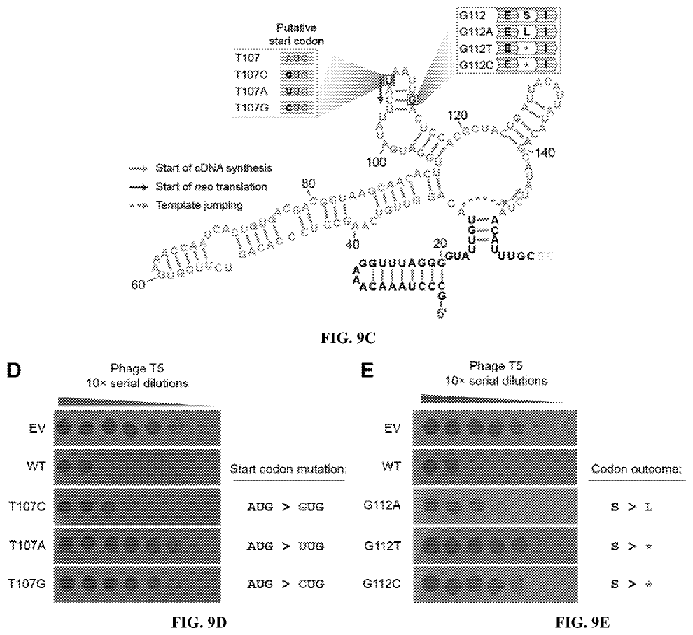

The sequence of the cDNA and it was determined that if this sequence was translated in silico, one out of three open reading frames (ORF) lacked any stop codons (FIGS. 3D and 9A). The concatenated RNA produced during phage infection might be translated to generate an antiviral polypeptide, supported by the presence of a predicted ribosome binding site upstream of the predicted start codon (FIGS. 3D and 9B), and by the observation that programmed template jumping adds one additional nucleotide during each round of cDNA synthesis (FIG. 2G). This activity generates a 120-bp cDNA repeat unit comprising exactly 40 sense codons, such that the reading frame would be preserved through each repeat to yield a continuous ORF (FIG. 3D).

To comprehensively test the hypothesis that translation of the continuous ORF within the concatenated RNA facilitates phage defense, region within SL3 that was not strongly conserved in sequence was identified, and single-bp mutations were introduced that would generate a synonymous, missense, or nonsense codon (FIG. 3E). While the synonymous and missense mutations had mild effects on defense activity, attributed to perturbation of the ncRNA secondary structure, the nonsense mutation completely abolished phage defense (FIG. 3F). The predicted start codon was mutated and mutation to the non-canonical GUG start codon partially preserved defense activity, but all other mutations were inactive (FIGS. 9C and 9D). To assess whether translation of multiple contiguous repeats of the ORF was necessary for phage defense, mutations that would introduce stop codons near the end of one full ORF repeat were tested and found to lead to a loss of defense (FIGS. 9C and 9E). Finally, three ncRNA loop regions were selected and insertions were designed ranging from 1-9 bp in length. Remarkably, all out-of-frame perturbations across three non-conserved loop regions, including minimal 1-bp insertions, caused a >10³-fold decrease in phage defense, while insertions of 3, 6, or 9 bp maintained near-WT activity levels (FIG. 3G).

Collectively, these experiments provided compelling genetic evidence for the existence and expression of a cryptic gene produced by RNA-templated concatenation of DNA repeats. Intriguingly, this de novo gene exhibits a heterogeneous length distribution and lacks any in-frame stop codons, and is referred to herein as neo (never-ending ORF).

### Example 4

### Neo-Encoded Polypeptides Induce Cell Dormancy

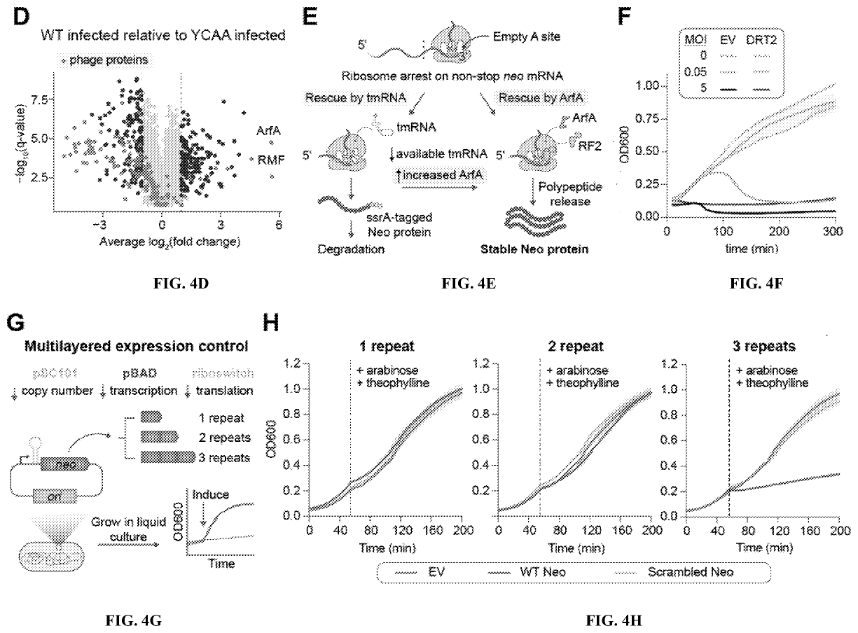

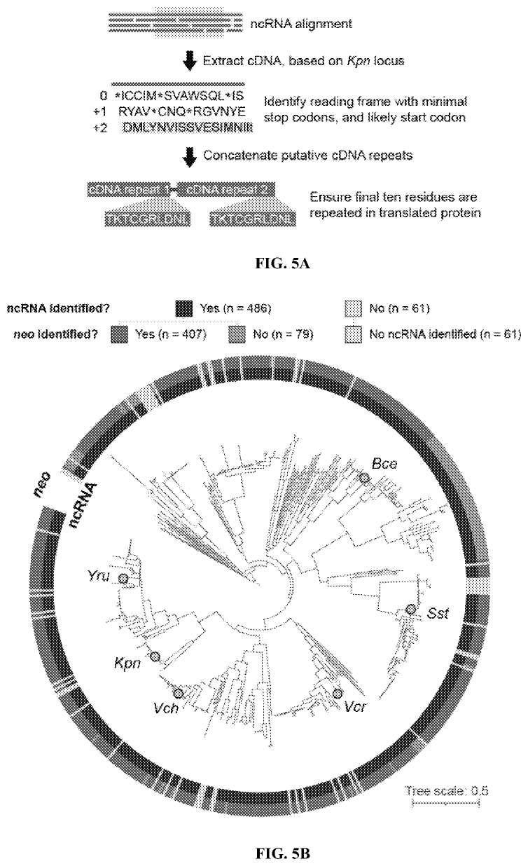

After analyzing the predicted amino acid composition of Neo, a custom protease cocktail was designed that would yield unique peptide fragments suitable for mass spectrometry (MS)-based proteomics (FIG. 10A). Proteins from KpnDRT2-expressing cells were extracted for liquid chromatography with tandem mass spectrometry (LC-MS/MS) analysis (FIG. 4A). Neo-derived peptides were detected in phage-infected cells that expressed the WT RT enzyme (FIG. 4B), and their abundances were substantial when compared to the rest of the E. coli proteome (FIG. 4C). These results provide concrete proof that neo mRNAs transcribed from concatenated cDNA genes are translated into protein.

Additional MS-based proteomics experiments were performed using a more standard trypsin-based digestion procedure to analyze the differential protein abundance between T5 phage-infected cells expressing WT or YCAA-mutant KpnDRT2. Phage proteins were widely depleted in WT cells, as likely for a protective immune response (FIG. 4D). On the host side, two significantly enriched cellular factors, ArfA and RMF, were notable due to their associations with ribosome stress and ribosome hibernation, respectively (FIG. 4D). ArfA (Alternative ribosome-rescue factor A) is a translation factor that specifically rescues ribosomes stalled on aberrant mRNAs lacking a stop codon, acting as an alternative to the tmRNA pathway that tags nascent polypeptide chains for degradation. ArfA is known to be specifically upregulated under conditions of tmRNA depletion, and its ribosome rescue activity in neo-expressing cells would elegantly resolve the conundrum of how stop codon-less neo mRNAs are nonetheless translated into functional proteins (FIG. 4E). RMF (Ribosome modulation factor) is a ribosome-associated protein that directs the assembly of 70S ribosomes into inactive 100S dimers during stationary phase, and is activated by the alarmone ppGpp, a known trigger of growth arrest and cellular dormancy. The upregulation of ArfA supports the likely mechanism by which Neo polypeptides are translated, and the induction of RMF suggests that Neo production is associated with cellular dormancy.

To investigate whether DRT2 uses abortive infection and programmed dormancy, phage infection assays were performed in liquid culture at varying multiplicities of infection (MOI). DRT2-expressing cultures survived T5 phage infection at low MOI, but infection at high MOI led to stalling of growth (FIG. 4F). Further analysis of cultures infected at high MOI revealed that DRT2 effectively blocked T5 replication (FIG. 10B), and that the growth-arrested cells remained viable (FIG. 10C), altogether supporting a mechanism of phage defense via programmed dormancy.

It was initially attempted to clone neo onto a standard inducible expression vector and test whether neo expression would be sufficient to trigger cellular dormancy. Yet repeated attempts to clone expression vectors with more than 2 repeats of WT neo proved unsuccessful, compared to scrambled control sequences that could be cloned with high efficiency, and the few colonies that emerged consistently exhibited frameshift mutations or lacked the neo insert altogether (FIGS. 10D and 10E). Neo may potently arrest cell growth, and leaky expression may have prevented the isolation of positive clones. This effect was only observed with Neo repeat lengths of 3 or more.

To circumvent this challenge, neo genes were placed on a low-copy vector under the control of a tightly regulated pBAD promoter and theophylline riboswitch (FIG. 4G). This multilayered strategy for control of neo expression, which evokes the elaborate regulation of neo expression by native DRT2 loci, enabled the isolation of the desired clones. The cells were then transformed with expression vectors encoding WT or scrambled neo with 1-3 repeats, and cell growth in liquid culture was monitored before and after inducing neo expression with arabinose and theophylline. Strikingly, only the 3-repeat WT Neo construct exhibited any growth defect compared to an empty vector control (FIG. 4H). To assess whether the growth-arrested cells could recover from dormancy, cells from the final time point of the liquid culture experiment were plated on solid media supplemented with either repressor (glucose) or inducer (arabinose and theophylline). Cells expressing 3-repeat WT Neo exhibited a ~10²-fold increase in colony-forming units when plated on repressor versus inducer (FIG. 10F), indicating strong recovery from Neo-induced dormancy.

### Example 5

### Neo Gene Synthesis and Neo Protein Toxicity

Equipped with a wealth of mechanistic information on the production of Neo protein by KpnDRT2, the evolutionary conservation of this gene synthesis strategy for antiviral defense was explored. Starting with a large phylogenetic tree of DRT2 homologs (FIG. 11A and Table 5), covariance models were used to annotate RT-associated ncRNAs and then the putative neo gene and Neo protein sequences were extracted based on the expected mechanism of template jumping and absence of in-frame stop codons ((FIG. 5A). The pipeline identified candidate ncRNAs and Neo proteins for the vast majority of DRT2 systems that were related to KpnDRT2 (FIG. 5B), revealing broad conservation of this unique mechanism for concatenated gene synthesis. Notably, sequence motifs expected to be critical for neo gene synthesis and expression, including ACA-1, ACA-2, and repeat junction-flanking promoter elements, were also strongly conserved across diverse homologs (FIG. 11B). Iterative generation of additional covariance models also enabled ncRNA prediction for more divergent DRT2 clades, but Neo protein annotation was more challenging, suggesting the possibility of alternative mechanisms of RCRT and neo gene expression (FIGS. 11A and 11C).

The amino acid sequences of diverse Neo proteins were examined in more detail. Bioinformatics analyses of multiple sequence alignments failed to identify any functional domains or similarities to known proteins, but they did reveal high-confidence predictions of α-helical secondary structural elements (FIG. 5C). Using a 3-repeat Neo sequence, the 3D protein fold was predicted using multiple independent methods, yielding a structure reminiscent of HEAT repeats and other alpha solenoids consisting of repeating antiparallel α-helices (FIGS. 5D and 11D). Helix-breaking proline residues were introduced into either the loop connecting two α-helices, or into the helices directly (FIG. 5D), and the effects of these perturbations on cell growth were assessed. Consistent with the structural model, insertions into either helix eliminated Neo-induced growth arrest, whereas the loop insertion mutant exhibited a dormancy phenotype similar to the WT Neo sequence (FIG. 5E).

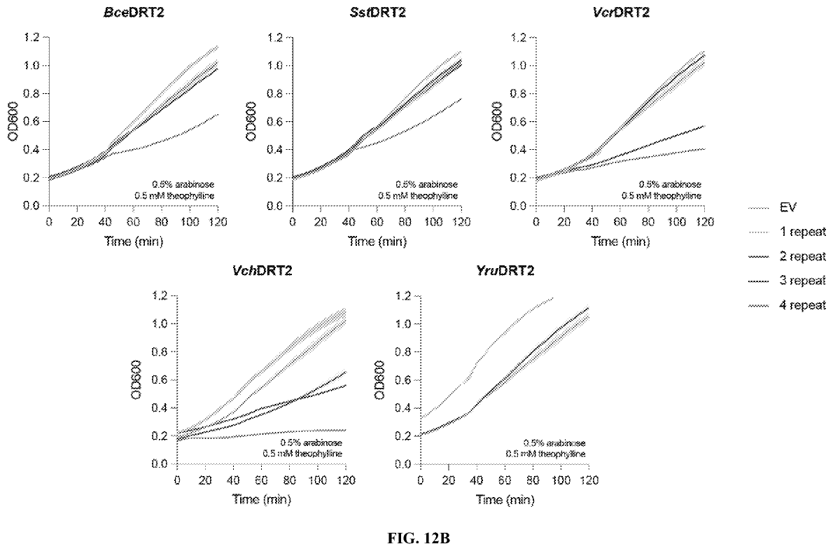

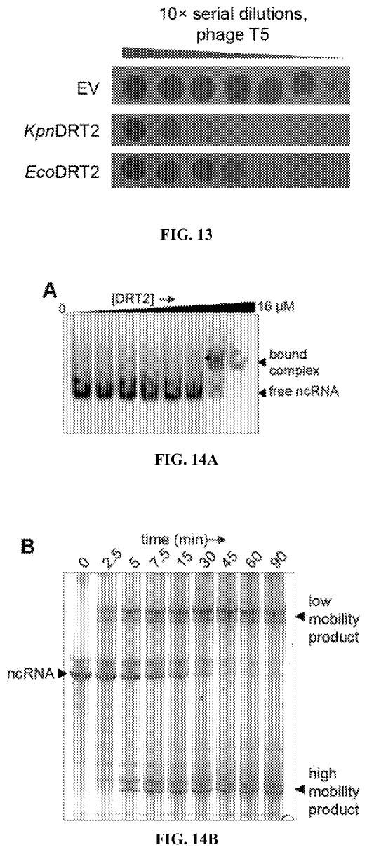

Five diverse DRT2 homologs were selected and clone (FIGS. 5B and 12A), and Nanopore sequencing of total DNA from cells transformed with DRT2 expression vectors was performed to assess the distribution of neo cDNA repeat lengths. Remarkably, nearly all of the tested systems exhibited RCRT upon heterologous expression in E. coli, and the cDNAs spanned a wide range of abundance levels and repeat lengths (FIG. 5F). The effect of recombinant expression of the Neo proteins predicted to be encoded by these concatenated cDNAs was also tested. In all cases, Neo homolog expression led to repeat length-dependent growth arrest, further confirming the requirement for cDNA repeat concatenation in the programmed dormancy mechanism to defend against phage infection (FIGS. 5G and 12B).

### Example 6

### Biochemical Reconstitution of Reverse Transcription

The ability of the KpnDRT2 reverse transcriptase (RT) to bind and reverse transcribe the non-coding RNA (ncRNA) template was tested in vitro using purified components. The RT enzyme was expressed in E. coli with an N-terminal His6 affinity tag and GST solubilization tag, as well as a C-terminal StrepII affinity tag. The enzyme was isolated from cell lysates by Ni-NTA affinity purification, followed by cleavage of the N-terminal tags by TEV protease and further purification by size-exclusion chromatography. The DRT2 ncRNA was generated by in vitro transcription followed by PAGE purification.

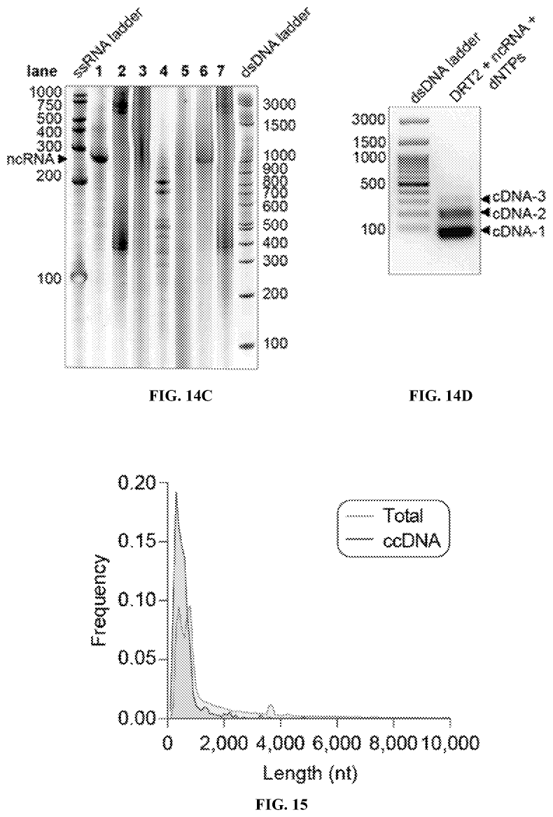

To test binding of the ncRNA by the RT, the purified ncRNA was mixed with increasing amounts of the RT in reaction buffer (20 mM HEPES pH 7.5, 5 mM MgCl₂, 5% glycerol, 200 mM NaCl, and 1 mM DTT). Samples were incubated at 37° C. for 15 minutes and analyzed by electrophoretic mobility shift assay (EMSA). The shift in mobility at higher concentrations of RT enzyme indicates binding of the ncRNA by the RT (FIG. 14A).

To test cDNA synthesis activity, the purified ncRNA and RT were incubated at 37° C. in reaction buffer (20 mM HEPES pH 7.5, 5 mM MgCl₂, 5% glycerol, 200 mM NaCl, 1 mM DTT) supplemented with 1 mM dNTPs, and reaction products were visualized after various incubation timepoints by urea-PAGE. High mobility and low mobility products are visible within 5 minutes of incubation, and the ncRNA is consumed in the reaction by 45 minutes of incubation (FIG. 14B). Treatment of the in vitro reverse transcription product with either RNase H or RNase A results in a shift in electrophoretic mobility, suggesting that the cDNA product is covalently linked to RNA (FIG. 14C). Treatment with double-stranded DNA-specific DNase (dsDNase) depletes the reaction product, suggesting that the cDNA product is potentially double-stranded (FIG. 14C). However, it is also possible that the enzyme is recognizing local regions of double-strandedness due to folding of a single-stranded product. Treatment with DNase I results in a single band with similar mobility to the initial ncRNA, which together with the results from RNase A treatment, provides further evidence that the cDNA product is covalently linked to the ncRNA (FIG. 14C). In contrast, treatment with proteinase K does not appreciably affect the mobility of the reaction product, indicating that the product is not covalently linked to protein. PCR of the reaction product using cDNA repeat-repeat junction-spanning primers reveals multiple bands of periodically increasing length, which represent one-repeat, two-repeat, and three-repeat concatenated cDNA products (FIG. 14D). In summary, rolling circle reverse transcription by DRT2 can be reconstituted in vitro from purified components, and the concatenated cDNA product is covalently linked to the ncRNA template.

### Example 7

### Reverse Transcription Processivity and Error Rate

The KpnDRT2 reverse transcriptase converts a short 120-nt template into a concatemeric cDNA product that can be as long as ~5 kb. Based on long-read sequencing, the median length of concatemeric cDNA (ccDNA) products is 479 nt (FIG. 15). This length distribution may reflect technical limitations of Nanopore sequencing rather than the true length distribution in cells, given the similarity between the ccDNA length distribution and that of the entire sequencing library from this experiment (“Total”). The median length for total reads was 833 nt. Additionally, the processivity of the RT may be even higher on a linear template that does not require template jumping for each successive round of 120-nt elongation.

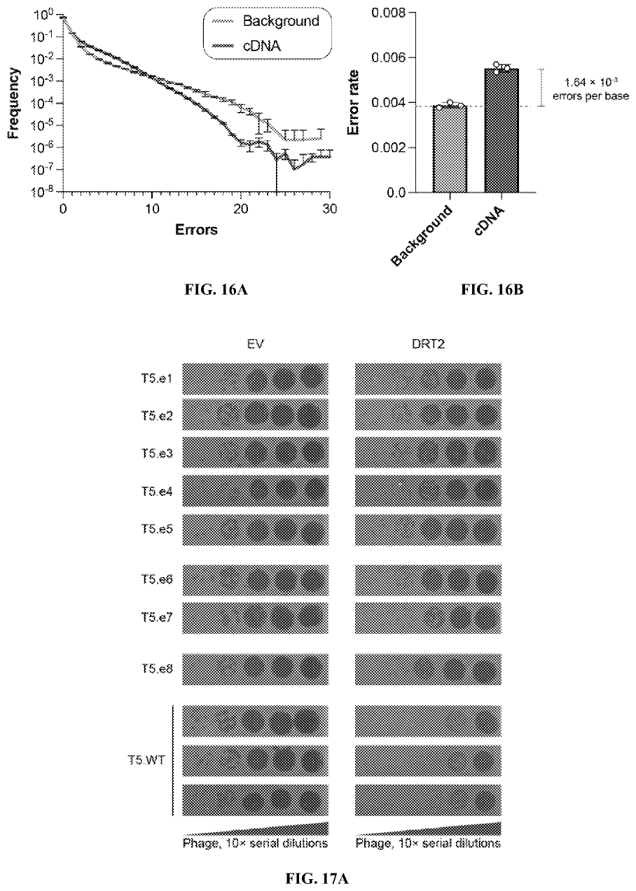

The error rate of the RT, calculated by analyzing error frequency in cDIP-seq reads aligning to the cDNA compared to background (reads aligning to the plasmid outside of the cDNA region, for matched input samples from cDIP-seq experiments), is estimated to be 1.64×10³ errors per base incorporated (FIG. 16). This corresponds to approximately 1 error per 610 bases incorporated.

### Example 8

### Triggering DRT2 Immune Activity

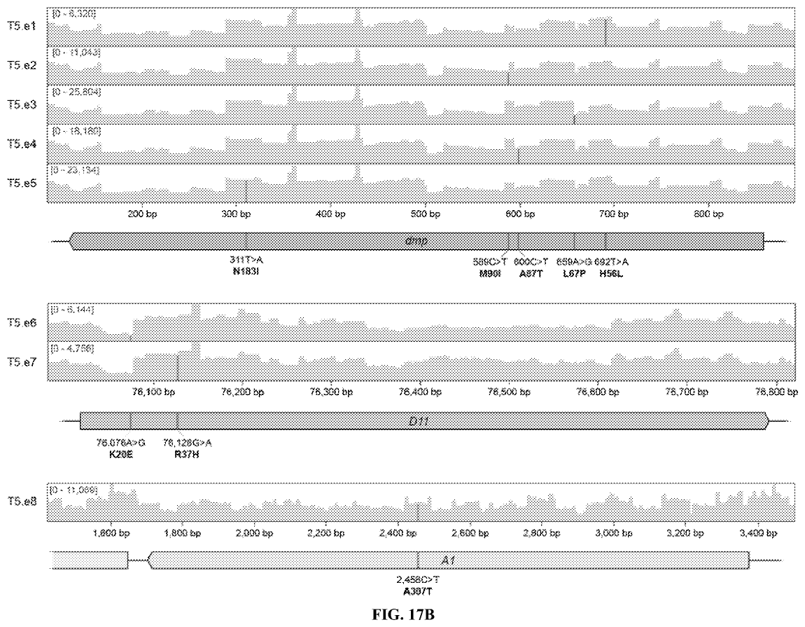

Plaques observed upon infection of DRT2-expressing E. coli with T5 phage would represent ‘escaper’ phage clones that had mutated to evade detection by the DRT2 defense system and could be used in order to identify potential triggers of DRT2-mediated immune activity. E. coli were infected with either empty vector (EV) or DRT2, and individual plaques from either infection were isolated and propagated. Eight plaques propagated from the DRT2 infection experiment represent potential T5 phage escaper clones, while three plaques propagated in parallel from the EV infection represent wild-type (WT) T5 controls. The defense sensitivity of these phages was validated by performing plaque assays on cells transformed with EV or DRT2. All eight escaper phages were not susceptible to DRT2 immune activity, whereas the three WT control phages were susceptible (FIG. 17A). The genomes of the escaper phages were sequenced in order to assess the presence of mutations underlying the immune evasion phenotype. Each escaper phage harbored one missense mutation: five phages were mutated in the dmp gene, two phages were mutated in the D11 gene, and one phage was mutated in the A1 gene (FIG. 17B). These genes encode proteins that are involved in nucleotide metabolism (dmp and A1) or phage genome replication (D11), suggesting that these activities may be involved in the increased cDNA production and second-strand cDNA synthesis observed during T5 phage infection of DRT2-expressing cells.

### Example 9

### Eukaryotic Activity

ncRNA binding and cDNA synthesis for DRT homologs with varied ncRNA designs in eukaryotic cells are interrogated using RIP-seq, c-DIP-seq, and long read sequencing. The ncRNA designs alter the position of the transcription terminator with respect to the final nucleotide of the ncRNA. Expression of the recombinant Neo proteins in the eukaryotic cells is monitored for toxicity phenotypes. Experiments use human codon-optimized DRT2 or Neo expression vectors, DRT2 systems contain an N-terminal BP-NLS and 3×FLAG tags, and various ncRNA constructs are all under U6 expression.

### Example 10

### ncRNA Sequence and Structure Modifications

ncRNA binding and cDNA synthesis are interrogated for DRT systems in which the ncRNA is modified both in sequence and structure. As described above, SL5 need not be retained for rolling circle reverse transcription and appears to be amenable to at least some level of insertion of random nucleotides, whereas the ACA motifs abutting and within SL2 and the structure of SL6 appear to be less amenable to modification. Additional modifications to the ncRNA include: mutations, deletions, and additions to the template region sequence; perturbations to or additional or deletions of secondary structures of the stem-loops or the template region, including SL3, SL4, and SL5; replacement of template regions or segments thereof with sequence of interest; and perturbations to or substitutions, additions, or deletions within SL7 and SL8.

### Example 11

### Probing Rolling Circle Reverse Transcription by Rational Mutagenesis

The highly efficient rolling-circle reverse transcription (RCRT) activity of DRTs represents a unique biochemical behavior that produces concatenated, repetitive cDNA molecules with precise junction sequences, expanding the diversity of products that can be generated by a single polymerase enzyme from its substrate. Intriguingly, it accomplishes this using a template that is not a closed circle, and thus differs from classic examples of rolling circle amplification associated with plasmid, phage, and viroid replication.

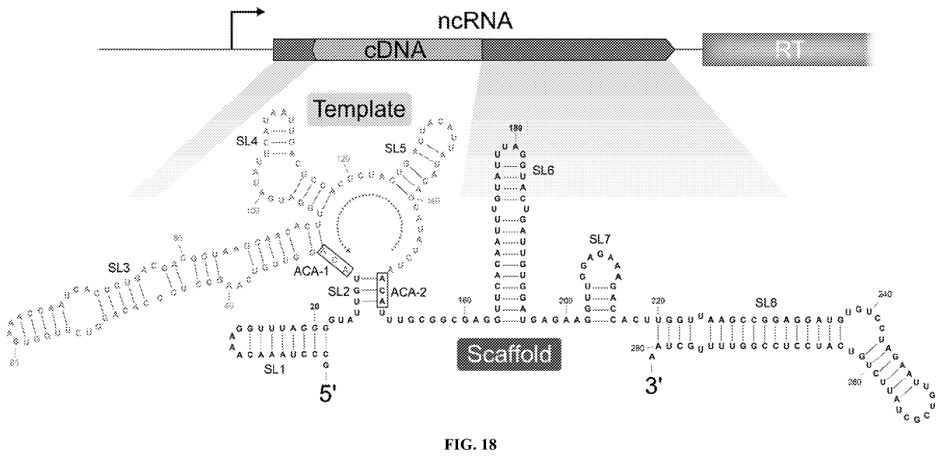

To assess the sequence requirements of rolling circle reverse transcription (RCRT) and the programmability of RCRT for the synthesis of custom DNA products, the non-coding RNA (ncRNA) template of the KpnDRT2 reverse transcriptase (RT) was rationally mutated and tested for reverse transcription in vivo (FIG. 18). Categories of engineered ncRNAs included: scaffold mutations, template jumping junction mutations, template sequence mutations, template structure mutations, and template length variants. Table 3 contains the ncRNA variant sequences tested in these experiments, and Table 4 contains the plasmid sequences that were used in cellular experiments testing these ncRNA variants. Note that the ncRNA is divided into two large domains, referred to as the ‘scaffold’ and ‘template’ (FIG. 18). The ‘template’ is used as a complementary template for RNA-templated DNA synthesis, whereas the ‘scaffold’ refers to all other nucleotides that do not template DNA synthesis but rather serve as a scaffold for reverse transcriptase enzyme recruitment.

To assess reverse transcription in vivo, E. coli str. K-12 substr. MG1655 was transformed with plasmids encoding the RT enzyme and mutant variants of its flanking ncRNA. Individual colonies were inoculated in liquid LB with chloramphenicol (25 μg mL⁻¹) and grown at 37° C. to OD₆₀₀ of 0.40. Phage T5 was added to 5 mL cultures at a multiplicity of infection (MOI) of 5, which was calculated as the ratio of phage PFU to bacterial colony forming units (CFU), assuming 8×10⁸ CFU in 1 mL culture at OD₆₀₀ of 1.0. After 40 min of infection, cells were harvested by centrifugation at 4,000×g for 5 min, the supernatant was removed, and DNA was immediately purified using Miniprep Kits (Qiagen). To sequence the reverse transcription products, ligation of Illumina adapters and conversion of ssDNA to dsDNA were performed using the xGen ssDNA & Low-Input DNA Library Prep Kit (IDT), and libraries were sequenced on an Illumina NextSeq 500 or an Element AVITI in paired-end mode with 150 cycles per end.

To analyze DNA sequencing data, datasets were processed using cutadapt (v4.2) to remove Illumina adapter sequences, trim low-quality ends from reads, and filter out reads shorter than 15 bp. Reads were mapped to combined reference files containing the E. coli genome and relevant plasmid sequence. Coverage tracks were generated and scaled according to sequencing depth before visualization. To assess total cDNA production, reads mapping to the KpnDRT2 cDNA locus were counted. To assess rolling circle reverse transcription, reads spanning the repeat-repeat junction in a custom reference sequence consisting of two concatenated cDNA repeats were counted. All counts were normalized for sequencing depth and calculated as counts per million reads (CPM).

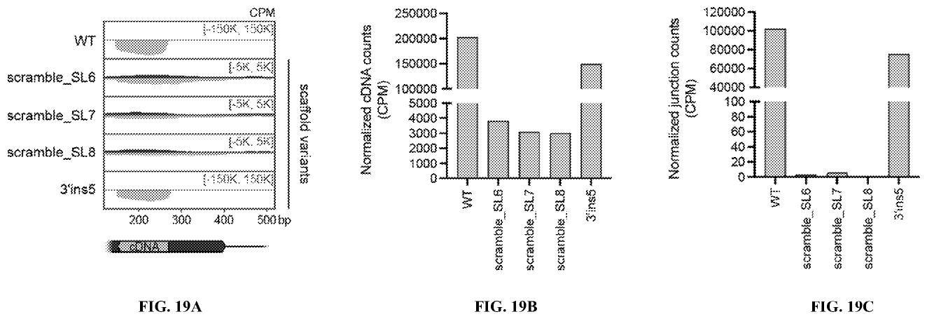

To probe scaffold regions, stem-loops (SL) within the scaffold were scrambled or the 3′ end was extended. Previous cDIP-seq data demonstrated that scrambling of SL1 or SL6 (a substrate referred to as “scramble_SL6”) resulted in a loss of reverse transcription activity. Here, scrambling of SL7 or SL8 resulted in a decrease in total cDNA production similar to scrambling of SL6, suggesting that these scaffold structural features facilitate reverse transcription (FIG. 19). To assess whether the ncRNA scaffold terminates at the precise 3′ end position observed in the WT system, a 5-nt sequence (5′-UAUUC-3′) was added to the 3′ end, generating a substrate referred to as “3′ins5.” Total cDNA production and RCRT were similar to WT, demonstrating that a 3′ extension of the ncRNA is tolerated by the reverse transcriptase (FIG. 19).

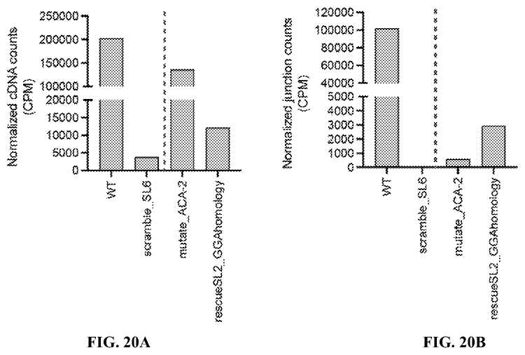

To probe whether the template jumping junction can be reprogrammed (e.g., the ACA-1 sequence motif and the ACA-2 sequence motif within the stem of SL2; see FIG. 18), ACA-1 and ACA-2 were mutated to GGA, with compensatory mutations introduced to the left side of the SL2 stem to maintain base pairing. Previous cDIP-seq data demonstrated that mutating ACA-2, which results in loss of both ACA homology and SL2 base pairing, led to WT levels of cDNA synthesis but a loss of programmed template jumping. Here, this result was recapitulated and furthermore showed that an engineered ncRNA in which ACA-1 and ACA-2 were both mutated to GGA, and the left side of the SL2 stem was mutated to complementary UCC, a substrate referred to as “rescueSL2_GGAhomology,” remained competent for both cDNA synthesis and programmed template jumping, albeit at lower levels than WT (FIG. 20). These results demonstrate that homology between ACA motifs, for RCRT in the WT system, can be reprogrammed to accommodate new sequence motifs, so long as homology in this region and SL2 base pairing are maintained.

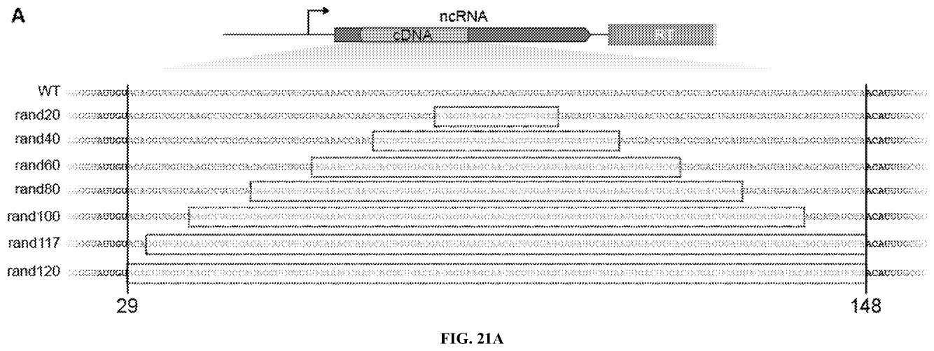

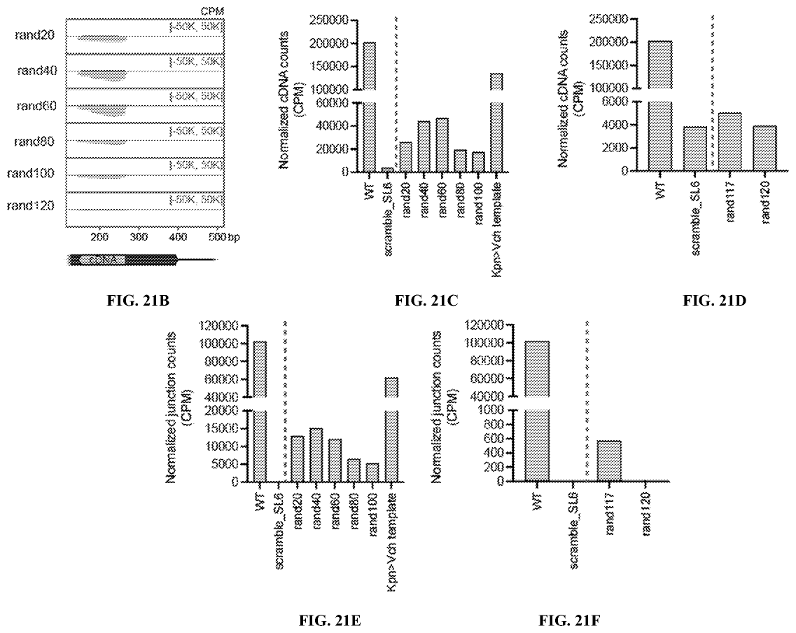

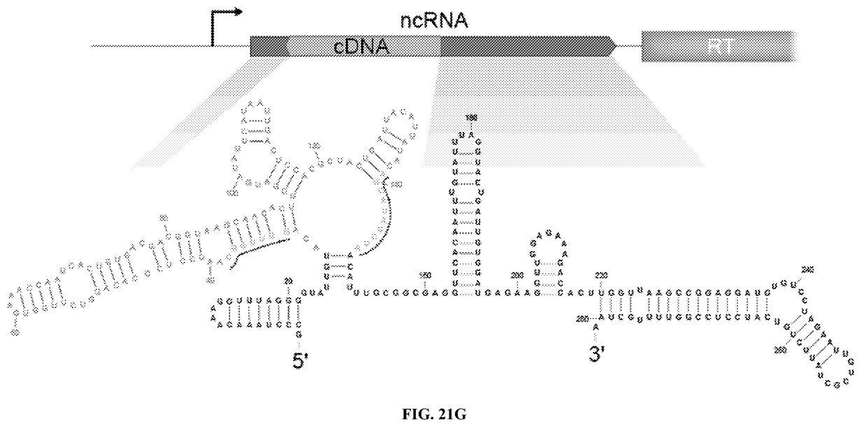

To probe template sequence, the 120-nt template sequence was sequentially mutated to unrelated (‘randomized’) sequences in multiples of 20 nt, starting at the template's midpoint between A88 and A89, and simultaneously extending upstream/downstream. FIG. 21A provides a schematic of the various substrates, including additional ones described below; these substrates are referred to as “rand20”, “rand40”, “rand60”, “rand80”, and “rand100.” Total cDNA production and RCRT were maintained at levels well above baseline (compared to SL6-8 mutants) with mutations of the 120-nt template region ranging from 20 to 100-nt in size (FIGS. 21B-21F). Mutation of the entire 120-nt template (substrate referred to as “rand120”) led to a substantial decrease in total cDNA production and near-complete loss of RCRT, but an alternative construct which mutated 117-nt of the template but left ACA-1 intact remained competent for RCRT (substrate referred to as “rand117”; FIGS. 21D and 21F). Yet, even with ACA-1 intact, the substantial decrease in both cDNA production and RCRT observed when increasing the mutagenized region from 100-nt to 117-nt suggests that sequence or structural elements for reverse transcription lie within the regions encompassing these 17-nt (FIG. 21). Mutation of either SL5 or the region between SL5 and the cDNA synthesis start site does not affect reverse transcription activity (described above), suggesting that the region responsible for the substantial difference in activity between rand100 and rand117 is most likely between positions G32-C38, within SL3.

An additional construct was also tested in which the template region of the KpnDRT2 ncRNA was replaced with that of the VchDRT2 ncRNA (substrate referred to as “Kpn>Vch template”). Although the template regions of these two homologs exhibit only 46% sequence identity, replacement of the KpnDRT2 template with the VchDRT2 template maintained near-WT total cDNA production and RCRT (FIGS. 21C and 21E). These results demonstrate that RCRT readily accommodates exogenous template sequences, so long as the homology between ACA motifs (or alternative, user-defined motifs) and SL2 base pairing are maintained. Reverse transcription may accommodate any sequence of interest within the template region, regardless of the compatibility for SL2-specific base pairing within the ACA motifs; however, the absence of SL2 compatibility may impact the degree of template jumping, rolling circle reverse transcription, and concatemeric cDNA production.

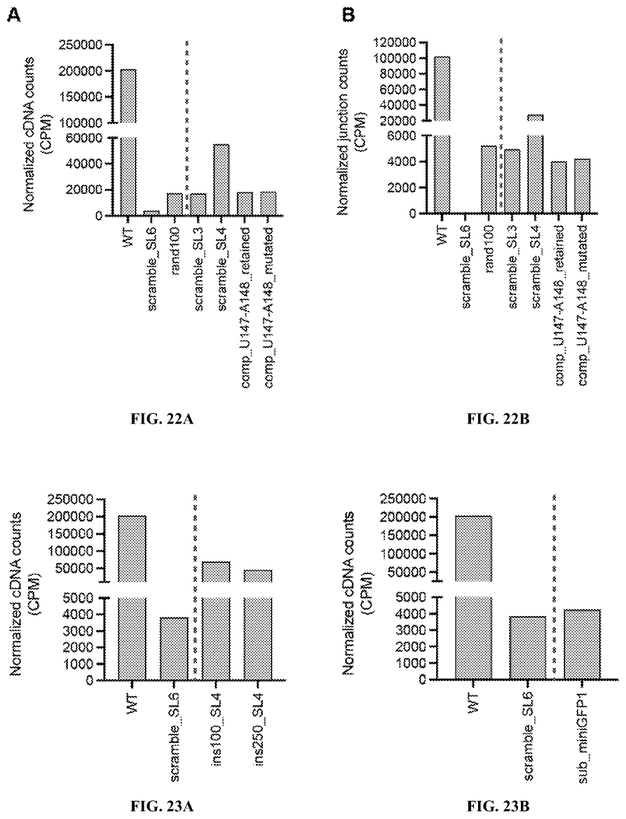

To probe potential structural RNA regions within the template region, scrambling of individual SLs or mutation of the entire template (excluding ACA-1) to its complementary sequence was tested. Previous cDIP-seq data demonstrated that scrambling SL5 permitted both reverse transcription and template jumping. Here, scrambled SL4 (substrate referred to as “scramble_SL4”) retained high levels of total cDNA production and RCRT, indicating that this template structural feature is amenable to mutation (FIG. 22). Scrambled SL3 (substrate referred to as “scramble_SL3”) exhibited lower cDNA production and RCRT than scrambled SL4, consistent with the notion that the region between positions G32-C38, within SL3, likely contains sequence or structural elements for reverse transcription (FIG. 21).

To test the effects of mutating the entire template to a new sequence but with a similar structure, the template (excluding ACA-1) was replaced with its complementary sequence. Here, two ‘complement’ variants were tested: one that retains the 2 bases at the 3′ end of the template region (U147-A148), and a second that mutates them to their complement; these two constructs are referred to as “comp_U147-A148_retained” and “comp_U147-A148_mutated”, respectively. These were tested in order to determine whether those two bases, which are well-conserved among DRT2 homologs, facilitate RCRT. Both variants demonstrated nearly identical levels of total cDNA production and RCRT, suggesting that the two bases at the 3′ end of the template region are non-essential for these processes (FIG. 22). Furthermore, both complement variants exhibited similar levels of total cDNA production and RCRT to the variant with 100-nt of the template randomized (referred to as “rand100”), suggesting that structural elements within the 100-nt region mutated in the “rand100” variant contribute minimally to cDNA synthesis and RCRT.

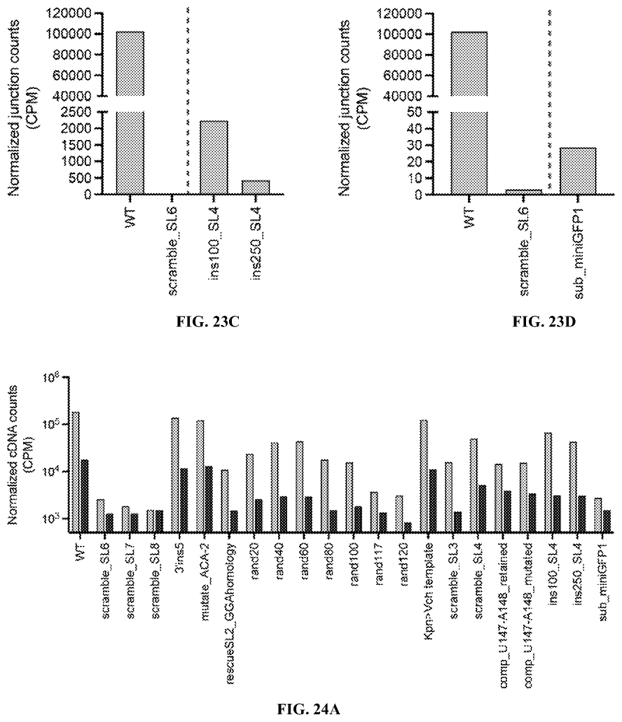

To probe the tolerance of the template sequence to variations in overall length, large insertions and substitutions of varying lengths and content were introduced. Previous plaque assay data demonstrated that insertions of 3, 6, and 9 nt in SL3 (between U59 and G60), SL4 (between U101 and A102), or SL5 (between C131 and A132) retained phage defense activity and therefore could be inferred to retain RCRT. Here, ncRNA variants were tested with insertions of ‘randomized’ 100- and 250-nt sequences into SL4 (between U101 and A102), referred to as “ins100_SL4” and “ins250_SL4,” respectively. Both constructs remained highly competent for cDNA synthesis and RCRT (FIG. 23). Additionally, swapping the entire template sequence, excluding the ACA-1 sequence motif, with the full-length coding sequence of the miniGFP1 gene, a construct referred to as “sub_miniGFP1,” resulted in low but detectable levels of total cDNA production and RCRT (FIG. 23). These results demonstrate that the DRT2 ncRNA can be re-engineered to template the synthesis and programmed amplification of exogenous gene-sized cargos.

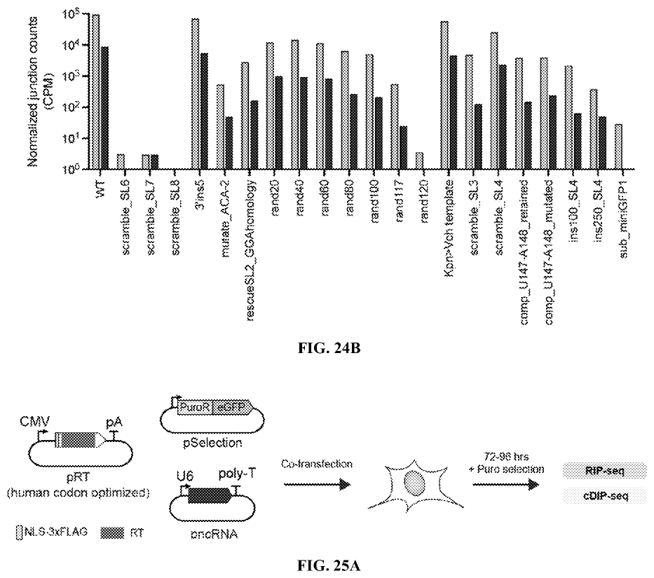

The DRT2 immune system is capable of robust generation of double-stranded DNA in the cell from a simple ncRNA molecule, which can be detected by strand-specific cDNA sequencing. The DNA high-throughput sequencing data from the experiments presented here that tested variant ncRNA sequences was analyzed for the presence of second-strand DNA synthesis and thus second-strand cDNA reads. Second-strand cDNA reads are detectable in nearly every cDNA template mutant tested (FIG. 24), indicating that engineered DRT2 ncRNAs remain competent for generating double-stranded DNA products.

### Example 12

### Reconstituting DRT2-Based cDNA Synthesis and Rolling Circle Reverse Transcription in Human Cells

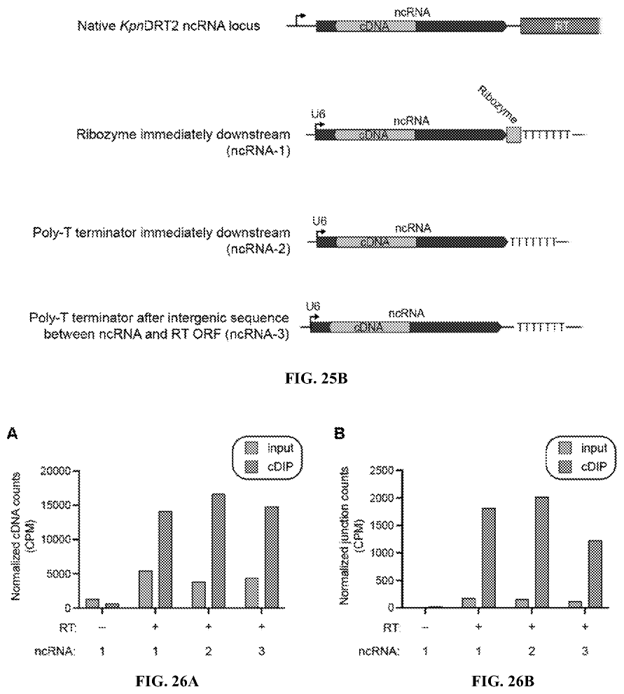

Experiments were designed to test the RNA-guided DNA synthesis activity of the DRT2 enzyme in human cells (FIG. 25A). In these experiments, the reverse transcriptase (RT) from the KpnDRT2 systems was human codon optimized and cloned into a pcDNA3.1 vector, including an encoded NLS and 3×-FLAG tag at the N-terminus of the ORF. This vector is referred to as pRT and drives the expression of the RT from a strong CMV promoter. Multiple different ncRNA architectures were designed and tested, in which the ncRNA is driven by a U6 promoter; the resulting vector is denoted pncRNA. In the first design (ncRNA-1, encoded by vector pSL7516), a ribozyme was encoded directly downstream of the ncRNA 3′ end, such that after ribozyme processing, the 5′ and 3′ boundaries of the final RNA product would match those of the mature KpnDRT2 ncRNA produced in E. coli. In the second design (ncRNA-2, encoded by vector pSL7517), a poly-T termination sequence was encoded immediately downstream of the DRT2 ncRNA 3′ end. In the third design (ncRNA-3, encoded by vector pSL7518), the sequence downstream of the 3′ end of the mature ncRNA from the native KpnDRT2 operon, up to the start codon of the RT ORF, was included in the ncRNA expression plasmid upstream of a poly-T termination sequence. All plasmid sequences can be found in Table 4.

To determine if the KpnDRT2 RT can bind its cognate ncRNA, synthesize cDNA, perform RCRT, and generate double-stranded DNA products in human cells, HEK293T cells were seeded in 10 cm dishes and transfected using common lipofection-based methods. Cells were co-transfected with a DRT2 expression plasmid (pRT), a ncRNA expression plasmid (pncRNA), and a plasmid encoding a drug resistance marker (pSelection). After 3 days of culturing with drug selection to enrich for transfected cells, cells were harvested, and reverse transcription activity was analyzed using RIP-seq and cDIP-seq methods. Total cDNA production was quantified by aligning cDIP-seq reads to the ncRNA expression plasmid reference and counting reads aligning to the cDNA locus. RCRT was quantified by aligning cDIP-seq reads to a reference file containing two concatenated cDNA repeats, and counting alignments spanning the repeat-repeat junction. These steps were performed for both the IP samples and corresponding input controls.

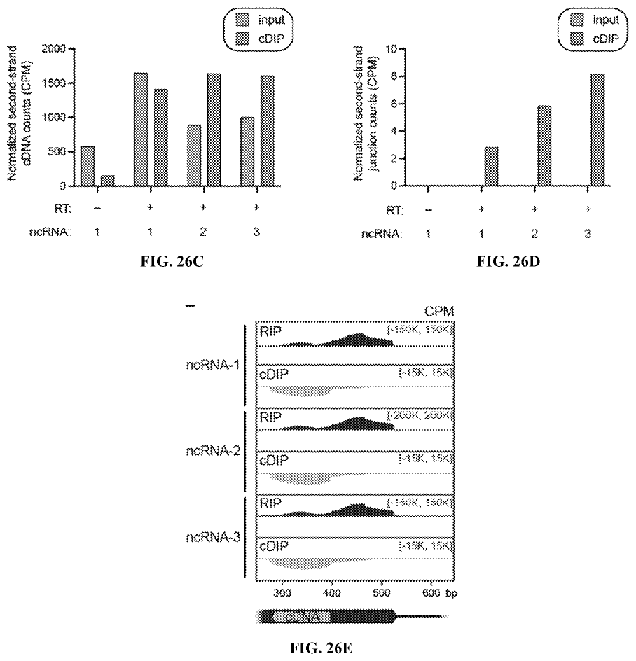

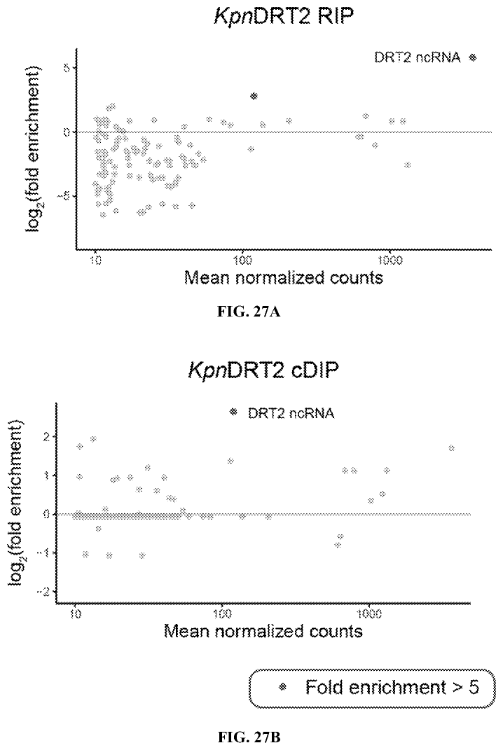

Strong enrichment of the KpnDRT2 cDNA was observed with cDIP-seq experiments across all tested ncRNA expression designs, compared to a control condition in which the RT expression plasmid was omitted (FIG. 26A). Furthermore, robust rolling circle reverse transcription (RCRT) activity was observed, as evidenced by the presence of abundant repeat-repeat junction-spanning reads in all conditions, except for the control no-RT condition (FIG. 26B). Reads corresponding to second-strand cDNA were also detectable, although they showed less cDIP-seq enrichment compared to first-strand cDNA reads (FIGS. 26C-26D). This is consistent with the prior findings in E. coli, and likely attributable to a lower affinity of the RT for double-stranded cDNA than single-stranded cDNA. When the RIP-seq and cDIP-seq reads were mapped to the ncRNA locus, strong sequencing coverage was found across the exact same ncRNA and cDNA regions as previously observed from RIP-seq and cDIP-seq experiments in E. coli, demonstrating that the RT enzyme retains the same ability to strongly bind its associated ncRNA and use this RNA for RNA-templated DNA synthesis of a chemically well-defined DNA molecule. Altogether, these results indicate that KpnDRT2 is active in generating concatemeric cDNA products in human cells, and that second-strand synthesis occurs at low levels in human cells without the requirement for other E. coli or phage-derived factors.

To assess substrate specificity of KpnDRT2 in human cells, RIP-seq and cDIP-seq reads were aligned to combined reference files comprising the human genome and KpnRT and ncRNA expression plasmids. Reads aligning to annotated transcripts were counted in order to assess the RIP-seq and cDIP-seq enrichment of the ncRNA locus, compared to other loci transcriptome-wide. The KpnDRT2 ncRNA was the most strongly enriched locus in both RIP-seq and cDIP-seq experiments (FIG. 27), indicating a high degree of substrate specificity between the RT and its cognate ncRNA.

### Example 13

### Rolling Circle Reverse Transcription (RCRT) and the Programmability of RCRT for the Synthesis of Custom DNA Products

To assess the sequence constraints of rolling circle reverse transcription (RCRT) and the programmability of RCRT for the synthesis of custom DNA products, the non-coding RNA (ncRNA) template of the KpnDRT2 reverse transcriptase (RT), FIG. 18, was rationally mutated and tested for reverse transcription in vivo. Categories of engineered ncRNAs included: 3′ end truncation, template jumping junction mutations, and template length variants. Table 3 contains the ncRNA variant sequences tested in these experiments, and Table 4 contains the plasmid sequences that were used in cellular experiments testing these ncRNA variants.

Reverse transcription in vivo was assessed as in Example 11. Total cDNA production counts and RCRT counts are shown in comparison to the wild-type (“WT”) system, representing baseline activity, as well as a system with a catalytically inactive RT (“YCAA”), representing background noise from the assay.

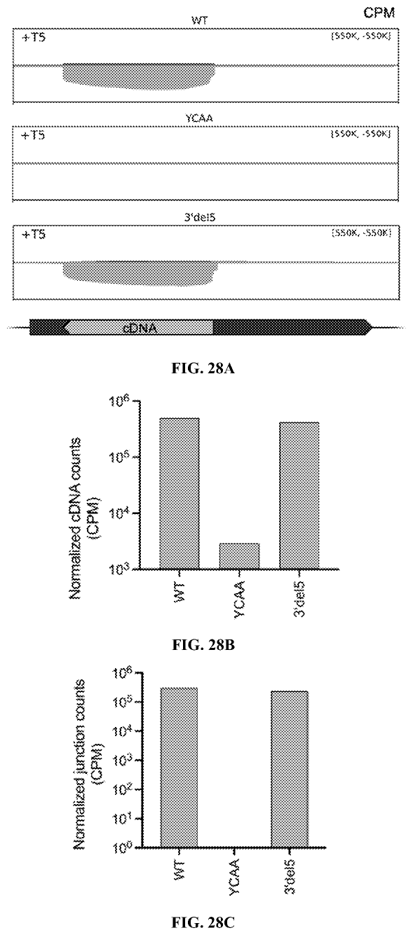

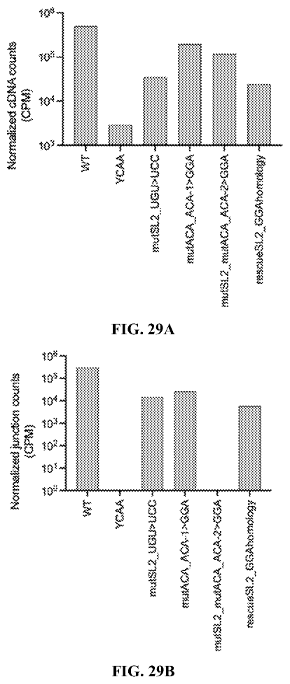

To assess the requirement for the ncRNA scaffold to terminate at the precise 3′ end position observed in the WT system, the last 5 bases of the scaffold were deleted, generating a substrate referred to as “3′del5”. Total cDNA production and RCRT were similar to WT, indicating that 3′ truncation of the ncRNA is tolerated for reverse transcription (FIG. 28).

To probe the contributions of sequences at the template jumping junction (e.g., the left half of the SL2 stem, the ACA-1 sequence motif, and the ACA-2 sequence motif, see FIG. 18), the left half of the SL2 stem was mutated to UCC, or ACA-1 or ACA-2 were individually mutated to GGA. As demonstrated in Example 11, an engineered ncRNA in which ACA-1 and ACA-2 were both mutated to GGA, and the left side of the SL2 stem was mutated to complementary UCC, remained competent for both cDNA synthesis and programmed template jumping, albeit at lower levels than WT (previous filing). This result was recapitulated (substrate referred to as “rescueSL2_GGAhomology”) and mutation of ACA-1 alone (substrate referred to as “mutACA_ACA-1>GGA”), or the left half of the SL2 stem alone (substrate referred to as “mutSL2_UGU>UCC”), resulted in lower, but still detectable levels of cDNA synthesis and RCRT (FIG. 29). Meanwhile, mutation of ACA-2 alone (substrate referred to as “mutSL2_mutACA_ACA-2>GGA”) decreased cDNA synthesis while completely abolishing RCRT (FIG. 29). These results demonstrate that individual mutations leading to a loss of SL2 base pairing or ACA motif microhomology are partially tolerated for RCRT, but a mutation that affects both SL2 base pairing and ACA motif microhomology (e.g., mutation of ACA-2) substantially negatively affect RCRT. The effect of such a mutation can be rescued by introducing compensatory mutations that restore both SL2 base pairing and ACA motif microhomology.

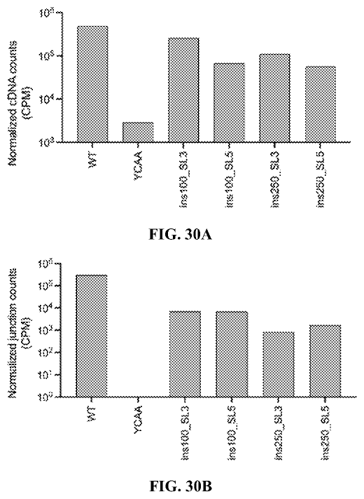

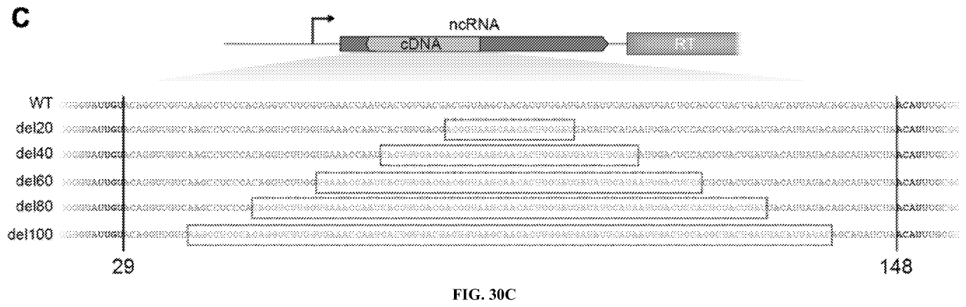

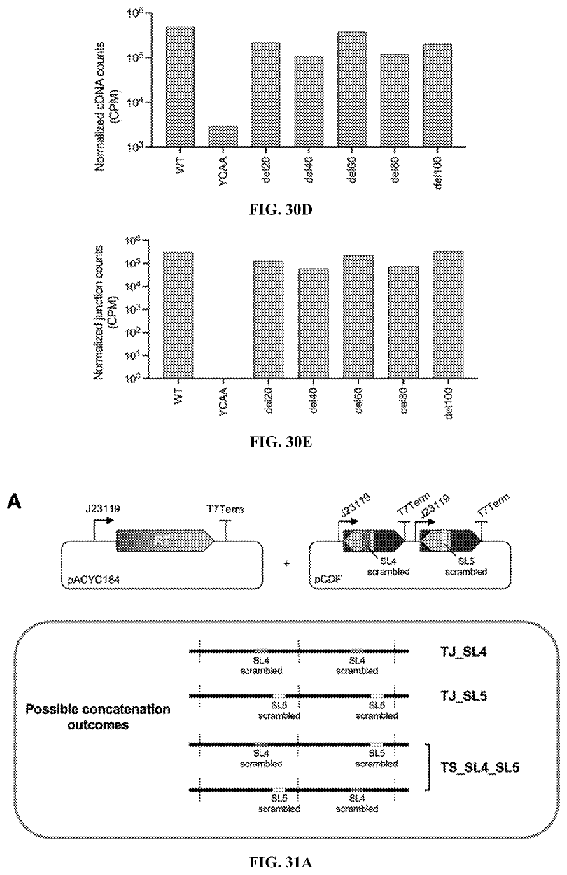

To probe the tolerance of the template sequence to variations in overall length, insertions and deletions of varying lengths were introduced. As shown in Example 11, ncRNA variants with insertions of ‘randomized’ 100- and 250-nt sequences into SL4 (between U101 and A102) retained cDNA synthesis and RCRT activity. Similar insertions into SL3 (between U59 and G60) and SL5 (between C131 and A132), referred to as “ins100_SL3”, “ins100_SL5”, “ins250_SL3”, and “ins250_SL5” were also tested. All four insertion variants retained cDNA synthesis and RCRT activity (FIGS. 30A-30B). The tolerance of the template sequence to deletions was also tested by systematically deleting bases in multiples of 20 nt, starting at the template's midpoint between A88 and A89 and simultaneously extending upstream/downstream. FIG. 30C provides a schematic of the various substrates; these substrates are referred to as “del20”, “del40”, “de160”, “del80”, and “del100”. Across all variants tested, total cDNA production and RCRT were maintained at near-WT levels (FIGS. 30D-30E). These data demonstrate that the ncRNA template can be substantially shortened or lengthened while remaining competent for cDNA synthesis and RCRT.

### Example 14

### Template Jumping Versus Template Switching

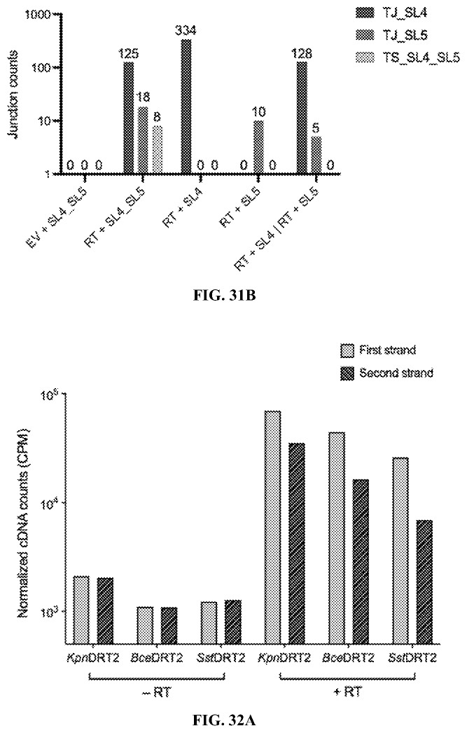

The data indicated that RCRT results from template jumping that is mediated by structural and sequence features of the ncRNA template, including the SL2 stem and ACA motif microhomology. While jumping from the end to the beginning of the template region may occur in cis (e.g., within the same molecule of ncRNA), it is also reasonable to consider that template jumping may occur in trans (e.g., between distinct molecules of ncRNA). Here “template jumping” refers to jumping in cis, and “template switching” refers to jumping in trans. To assess the occurrence of template jumping versus template switching, cells were transformed with two plasmids, one encoding the KpnDRT2 RT, and the other encoding two distinct KpnDRT2 ncRNA variants (“SL4_scrambled” and “SL5_scrambled”) (FIG. 31A). As in the above experiments assessing reverse transcription in vivo, cDNA synthesis was stimulated by infecting cells with T5 phage, and then harvesting DNA for library preparation and high-throughput sequencing. Template jumping and template switching were quantified by aligning sequencing reads to a reference sequence containing two concatenated cDNA repeats. Three possible concatenation outcomes were evaluated: 1) concatenation of “SL4_scrambled” to “SL4_scrambled”, referred to as “TJ_SL4”; 2) concatenation of “SL5_scrambled” to “SL5_scrambled”, referred to as “TJ_SL5”; 3) concatenation of “SL4 scrambled” to “SL5_scrambled”, or “SL5_scrambled” to “SL4_scrambled”, referred to as “TS_SL4_SL5”. All three outcomes were observed in cells co-expressing the KpnDRT2 RT, SL4_scrambled ncRNA, and SL5_scrambled ncRNA (condition referred to as “RT+SL4_SL5”), while none of the three outcomes were observed in a control experiment lacking the RT (“EV+SL4_SL5”) (FIG. 31B). Additional conditions were tested in which cells co-expressed the RT with either SL4_scrambled alone or SL5_scrambled alone (“RT+SL4” or “RT+SL5”, respectively). In each condition, only the concatenation outcome involving the expressed ncRNA variant was observed (FIG. 31B). To control for the possibility of template switching junctions arising from PCR recombination or other technical artifacts, an additional condition was tested in which DNA from the “RT+SL4” and “RT+SL5” conditions was mixed prior to library preparation and sequencing (“RT+SL4|RT+SL5”). Template jumping junctions were observed in this sample, but no template switching junctions were observed (FIG. 31B), indicating that the template switching observed in the RT+SL4_SL5 condition occurs at levels above background. These data demonstrate that RCRT may result from either template jumping or template switching.

### Example 15

### cDNA Synthesis and RCRT Activity of Diverse DRT2 Homologs in Human Cells

Example 12 demonstrated cDNA synthesis and RCRT activity for an engineered KpnDRT2 system expressed in human cells. To evaluate the activity of additional homologous DRT2 systems in human cells, the RTs from DRT2 systems encoded by Burkholderia cepacia (BceDRT2) or Stutzerimonas stutzeri (SstDRT2) were human codon-optimized and subcloned onto pcDNA3.1 vectors downstream of a CMV promoter, with an NLS and 3×-FLAG tag appended to the N-terminus of each RT. The corresponding ncRNAs from these systems were also subcloned onto expression plasmids downstream of a U6 promoter, including the intergenic sequence from the native operons between the 3′ end of the ncRNA and the start codon of the RT ORF. All plasmid sequences can be found in Table 4.

As previously done with KpnDRT2, HEK293T cells were seeded in 10 cm dishes and transfected using common lipofection-based methods. Cells were co-transfected with a DRT2 expression plasmid, the corresponding ncRNA expression plasmid, and a plasmid encoding a drug resistance marker. After 3 days of culturing with drug selection to enrich for transfected cells, cells were harvested, and reverse transcription activity was analyzed using cDIP-seq methods as described above. Total cDNA production was quantified by aligning cDIP-seq reads to the ncRNA expression plasmid reference and counting reads aligning to the cDNA locus. RCRT was quantified by aligning cDIP-seq reads to a reference file containing two concatenated cDNA repeats, and counting alignments spanning the repeat-repeat junction. These steps were also performed for control samples not transfected with the DRT2 expression plasmid (conditions referred to as “−RT”).

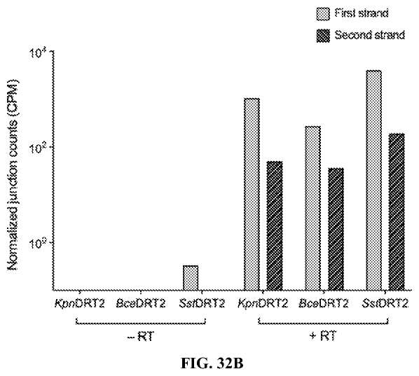

Both BceDRT2 and SstDRT2 showed clear evidence of RT activity in human cells, although total cDNA production was lower compared to KpnDRT2 (FIG. 32A). Both homologs also exhibited RCRT activity in human cells, with SstDRT2 demonstrating the highest RCRT activity out of all tested homologs (FIG. 32B). Reads corresponding to second-strand cDNA were also well above background, although they were lower in abundance than first-strand cDNA reads (FIG. 32B). Altogether, these results identify additional DRT2 homologs that are active for cDNA synthesis and RCRT in human cells, and reveal a high level of baseline RCRT activity for SstDRT2 in human cells compared to other tested homologs.

TABLE 1
Sequences of DRT2 homologs

| | | | CDNA |
|---|---|---|---|
| Species of origin | DRT amino acid sequence | ncRNA sequence | sequence |
| Vibrio cholerae | MSSNKPLWYRSRGYLHFDLPVSINKAKN | GTCGCTGATCAAAAGGAG | TAGAAATG |
| (Vch) | LVTNPDLVSKHAFFPLISYTVESKKISKNK | CTTAACAGGTTGTCAAGT | GATTATGCT |
| | DTRKIDVAVKQRPIAYSSHMDSHIYAFYA | CCAATCGGCACGCCACCA | ATAACTATA |
| | NLLSKKYEAKLKSAGLTESILAFRGLGKS | AGGCAAGCTGAGCAGAT | ACCGTGGA |
| | NIDFAFEAFEQIKTMGECSAVALDFSKFF | AGACAGCATGAACTGGA | GGTATCTAT |
| | DTLDHDLLKQSWANLLGEKKLPSDHFNV | ACAGGGTCTTCATAGATA | GAAGACCC |
| | FKSITKFSQVNKEELYKEFGISPNNPKNGR | CCTCCACGGTTATAGTTA | TGTTCCAGT |
| | NRVCTAQEFRDVVRNKKLIKTNKGSFGIP | TAGCATAATCCATTTCTA | TCATGCTGT |
| | QGSPISALLSNIYMFEFDAEMKRYMDIHG | ACATGACGCGGCTAGAGT | CTATCTGCT |
| | GKYYRYCDDMLFIVPAPLRDKVAGYAQE | TTTAGGACTCGTGGATGG | CAGCTTGCC |
| | QVKNLKVSINPKKTELRTFSSQGGILVAD | TTGTTTTAACAACTTGGTT | TTGGTGGCG |
| | QMLQYLGFMFDGENVYIRSSSLARYSEK | AAGCCGAGGTAAGGCGT | TGCCGATTG |
| | MKRGIRLAKKTMDKYNAVRVAKGLPEK | GAGGAATTGCCGCAGTTC | GACTTGAC |
| | SLYKRKLYSRYTHLGGRNFVTYGLRAAS | TAAGTCTGCCTCGGTTTT | AACCTGT |
| | AMQSKTIKRQLKPLWRRFHEELEKET | GCTAG (SEQ ID NO: 825) | (SEQ ID NO: |
| | (SEQ ID NO: 1) | | 835) |
| Escherichia coli | MNNDYPWFRKRGYLHFDEPVSLKKAVK | GCCCTAAACAAAGGTTTA | TAGATATGC |
| (Eco) | YVSSPEKIIKHSFLPFLSFEVKSFKIKKDKS | GGGGTATTGTACAGGTTG | TGTATAATG |
| | TKQLSKTEKLRPIAYSSHLDSHIYAFYAEY | TCAAGCCTCCCACAGTTC | TAATCAGTA |
| | LTGHYELLIQKNNLHENILAFRSLNKSNIE | TTGGTGAAACCAATCACT | GCGTGGAG |
| | FAKRAFDTITEMGECSAVALDLSGFFDNL | GTGACGACGGTAAGCAAC | TCAATTATG |
| | DHQILKHQWCKVIGTEALPQDHFAIYKSI | ACTTGGATGATATTCATA | AATATCATC |
| | TRYSKVDKNRAYEILGISKNNPKYNRRKI | ATTGACTCCACGCTACTG | CAAGTGTTG |
| | CTPVDFRNKIRKNGLIIVNKAQKGIPQGSP | ATTACATTATACAGCATA | CTTACCGTC |
| | ISALLSNIYMLDFDIEMRDYAQELGGHYY | TCTAACATTTGCGGCGAG | GTCACAGT |
| | RYCDDMLFIVPTKYNKTLAGDVAQRIKN | GTTCACAATTTGTATTTA | GATTGGTTT |
| | LKVELNTKKTEIRDFIYKDSTLVANMPLQ | GGTACTGATTGTGGATGA | CACCAAGA |
| | YLGFIFDGNNIFLRSSSLARYSERMKRGV | GAAGGTTGGAGAAAGAC | ACTGTGGG |
| | RLAKATMDSKNRIRQIKGEALKALFKKK | CACTTGGTTAAGCCGGAG | AGGCTTGA |
| | LYARYSHIGRRNFLTYGYRAAKIMNSRTI | GATGTGTCCTAGAATTGT | CAACCTGT |
| | KRQLKPLQKRLENEILK (SEQ ID NO: 2) | CGCTATTCTGTCATCCTCC | (SEQ ID NO: |
| | | GGTTTTGCTAA (SEQ ID | 836) |
| | | NO: 826) | |
| Klebsiella | MNNDDYPWFRKRGYLHFDEPVSLKKAV | GCCCTAAACAAAGGTTTA | TAGATATGC |
| pneumoniae (Kpn) | KYVSSPEKIIKHSFLPFLSFEVKSFKIKKDK | GGGGTATTGTACAGGTTG | TGTATAATG |
| | STKQLSKTEKLRPIAYSSHLDSHIYAFYAE | TCAAGCCTCCCACAGGTC | TAATCAGTA |
| | YLTGHYELLIQENNLHENILAFRSLNKSNI | TTGGTGAAACCAATCACT | GCGTGGAG |
| | EFAKRAFDTITEMGECSAVALDLSGFFDN | GTGACGACGGTAAGCAAC | TCAATTATG |
| | LDHQILKHQWCKVIGTEALPQDHFAIYKS | ACTTGGATGATATTCATA | AATATCATC |
| | ITRYSKVDKNRAYEILGISKNNPKYNRRKI | ATTGACTCCACGCTACTG | CAAGTGTTG |
| | CTPVDFRNKIRKNGLIIVNNSQKGIPQGSPI | ATTACATTATACAGCATA | CTTACCGTC |
| | SALLSNIYMLDFDIEMRDYAQERGGHYY | TCTAACATTTGCGGCGAG | GTCACAGT |
| | RYCDDMLFIVPTKYNKTLAGDVAQRIKH | GTTCACAATTTGTATTTA | GATTGGTTT |
| | LKVELNTKKTEIRDFIYKDSTLVANMPLQ | GGTACTGATTGTGGATGA | CACCAAGA |
| | YLGFIFDGSNILLRSSSLARYSERMKRGVR | GAAGGTTGGAGAAAGAC | CCTGTGGG |
| | LAKATMDSKNRIRENKGEALKALFKKKL | CACTTGGTTAAGCCGGAG | AGGCTTGA |
| | YARYSHIGRRNFLTYGYRAAKIMNSKAIK | GATGTGTCCTAGAATTGT | CAACCTGT |
| | RQLKPLQKRLENEILK (SEQ ID NO: 3) | CGCTATTCTGTCATCCTCC | (SEQ ID NO: |
| | | GGTTTTGCTAA (SEQ ID | 837) |
| | | NO: 827) | |
| Yersinia ruckeri | MESREHPWYRRRGYLHFDKPISLEHALDI | GCCCTACGCATCGGCGTA | TAGAAACA |
| (Yru) | VSNPDLIATHSFLPFITFTSASYKIKQDKST | GGGGTGATGTACAGTTTG | CTGTATAAT |
| | SAINKILKERPIAYSSHVDSHIYSYYAAILS | TCAACTCCCGGCCAGCGA | GTAATCAGT |
| | ELYENELRAHNVSKNVLAFRTLNKSNINF | CCTTCGCTGAACACCGAC | ATAATGGA |
| | AYEAFQEIKIRGNCSAVALDLSKFFDTIDH | CAGAGGCTGACTAGCAGG | GACAATAA |
| | GFLKDAWCKLLATDKLPEDHYAVFKAIT | TGGATGATATTCATTATT | TGAATATCA |
| | KYSKVDKSTIYDLMGIPKNNAKYTKKTR | GTCTCCATTATACTGATT | TCCACCTGC |
| | KQICTFVEFRDKVRKGKLIVSNKAGFGIP | ACATTATACAGTGTTTCT | TAGTCAGCC |
| | QGSPISALLSNIYMLNFDIEMDEYVSSLGG | AACATTTGCGGTGAAATG | TCTGGTCGG |
| | VYFRYCDDMLFIVPTDEKNNIAGEAEKRL | CACTTTTGGACTACAAGT | TGTTCAGCG |
| | IKLKVSLNIKKTEIREFSVSPTKIRCDKPLQ | AGTCCATTGTGTATGAGA | AAGGTCGC |
| | YLGFLFDGENIFLRSSSLSRYSDRMKSGV | AGGTTGGATAAAGACCAC | TGGCCGGG |
| | RLAKATMKRKNKIRKLKGLSKKELFKEKI | TTTGTTAAGCCGGGGCAA | AGTTGACA |
| | YARYAHVGKRNFLTYGYRASRIMNSNSI | TGTATTTTGAACGACCGC | AACTGT |
| | RKQLKPLWERLQKEISK (SEQ ID NO: 4) | AGTTCAAAATTGCCCCGG | (SEQ ID NO: |
| | | TTTCACAAT (SEQ ID NO: | 838) |
| | | 828) | |
| Vibrio | MKNTSEHPWFTKRNYLHFDIPVSVHKAE | GCTTTACGCGATAGGCGT | TAGAAATG |
| crassostreae (Vcr) | RIASEPITVARHAFFPFISYSFQLTKLDRDT | GAAGGGCTGTGACAGATT | ATATATAAT |
| | STGKLYRKPKPRGVAYSSHVDSHIYAFYA | GTCAATGCCTTTATCCTTG | GTAATCTGC |
| | SQLSTLYEKQIEKLGLHESVLAFRKLDGK | CAGAGCCAAGATAAACA | GATGGAGA |
| | SNIHFADDAFKHISKFEDCSVIALDFSKFF | CATTGTCATGAAGCGTAA | CTATACAAT |
| | DTLDHQILKEEWRKLLGEDCLPKDHYNV | GATTACCATTGTATAGTC | GGTAATCTT |
| | FRSLTKFSTVDKCALFDLFDISQYNPKAN | TCCATCGCAGATTACATT | ACGCTTCAT |
| | GRNRVCSPSEFRQKVRGSGLIEQNPNGM | ATATATCATTTCTAATAG | GACAATGT |
| | MGIPQGSPISALLSNIYMLNFDAQMKSYV | ATCGCGGCGTGTGCCTTT | GTTTATCTT |
| | DSVDGKYFRYCDDMLFIVPSEHRDMIEKL | AGGGGCCAGATGATTGGT | GGCTCTGCA |
| | ANSKVTALKVKINSDKTEIRDFRIHDGKL | TAGGCCAGTTAGTTAAAG | AGGATAAA |
| | SSFDEHGKQKPLQYLGFLFDGHSIFLRSTA | CCCAAAGGGATATACGGA | GGCATTGA |
| | LARYSEKMKGGVKLAKKTMEKENRIRR | AAGGCCGCATGTCCACTC | CAATCTGT |
| | DKGEPEQTYLYKRKLHKLYTARGQRNFL | CCTTTGGGTTTTGCTAG | (SEQ ID NO: |
| | SYGYRAAKIMNSKSIRKQLKPL WQRFQD | (SEQ ID NO: 829) | 839) |
| | EVSK (SEQ ID NO: 5) | | |
| Alloalcanivorax | MAKESPSWFQRRTYLHFDRPVGAKKAER | GCCCCGTGGGTTCGCTGC | TATCAATCA |
| xenomutans (Axe) | VVSDPVEVARHSFFPFLTYNITSHKARKT | GGGGGAGCACTACAGGA | TGTACACTA |
| | EAGNMEMVAKERPIHYAAHMDSHIYSYY | TGTCAACTGACCTGCACT | TCACTGTGA |
| | AEILSELYEKKLSELGISSSVLAFRKLGKS | GGCGGCTAGCCGCTAGCA | GCACATTG |
| | NIEFAKDAFDIIKMKAPCVAVGYDVSKFF | CATGACAGTACGGCACCC | GAGAACTTT |
| | QKIDHAKLKATWVGLLDEEILPKDHHAV | ACGTGAAGACGTTGAATA | ATGAAAGT |
| | FRSLTKYAEMDRGKVYEILGLPKYNKKSI | GAGTGACTTTCATAAAGT | CACTCTATT |
| | GPRICSPSQFRETVRPSKEMYVNDRSVGIP | TCTCCAATGTGCTCACAG | CAACGTCTT |
| | QGSPISALLSNIYMINFDCAIAEVAKEAGA | TGATAGTGTACATGATTG | CACGTGGG |
| | YYCRYCDDILVVCDTENVVDFEDLIRREL | ATAACACCATGCGGCTCG | TGCCGTACT |
| | GKLKLDINEQKTDKARYRVSKGKVYCDR | CTGCGGGTAACCGCGGTT | GTCATGTGC |
| | PIQYLGFLFDGESVFLRSASLARYSERMR | AGAAGTGCAAAAGATGC | TAGCGGCT |
| | RGVHLAKKTKRKYNKMRVKRGEPIRRLY | ACTGGCTCATGCCGGCAG | AGCCGCCA |
| | KKKLYARYSHLGRRNFVRYGLRAADIME | AGGGATAATGGGATCGTC | GTGCAGGT |
| | SKEIRQQLKPLWRRLKEEMER (SEQ ID | GCAGTCCTACCTCTGCCG | CAGTTGAC |
| | NO: 6) | GTTTGGCTAG (SEQ ID NO: | ATCCTGT |
| | | 830) | (SEQ ID NO: |
| | | | 840) |
| Collimonas | MVEKYYLVNPGERSIDCRHLRSPGLLVHP | GCTCTACGTGTAGATGTA | TAGAAATG |
| arenae (Car) | EKVRLFKKKHPWFRRRGYRHFDNPIGFA | GAGGAATATTACAGGTTG | CTTCATACT |
| | TARKIVTSPHRVETHSFYPLIDFNATSIKV | TCAAGTCCACTGAGCCAG | ATTAATATC |
| | KREASTNQLKKIPKIRPICYAAHIDSHIYS | AAAGCAAGTTTGGCAAAG | ACTATGGA |
| | YYAYMLAKRYEQAVHENGLDTCVIAFRS | CAGATTGATAACAGAACT | GGCTTCCTT |
| | LKKSNIEFAADVFDEIKRRDNCHVICLDIK | GTGCCTGGATTCCGCAAG | GCGGAATC |
| | GFFDNLDHETLKNSWANILGQEKLPGDH | GAAGCCTCCATAGTGATA | CAGGCACA |
| | YAVFRSLTNHAKVGKTELYALLDISIHNP | TTAATAGTATGAAGCATT | GTTCTGTTA |
| | GGNSKRLCSVEDFREKVRKAKIIKRQTGT | TCTAACATATGCGGCTTG | TCAATCTGC |
| | KGIPQGSPISALLSNIYMLAFDIVINSYVQ | GCTCAACTTGTTGAGTGA | TTTGCCAAA |
| | KCGGRYFRYCDDMLLIVPAEEKNQALNL | GATGGTAGAGAAATACTA | CTTGCTTTC |
| | AEAEIKKLLLIIQSEKTEQRDFAVVAGKL | CTTGGTTAATCCGGGAGA | TGGCTCAGT |
| | QSSKPLQYLGFLFDGQRILLRSASLARYS | ACGGTCAATAGATTGCCG | GGACTTGA |
| | DRMRRGIRLAKATMKKKNEVRTANGDA | CCATCTTCGTTCTCCCGGT | CAACCTGT |
| | AKALFKRKIYKRYTCVGRRNFITYGYRA | TTGCTAG (SEQ ID NO: 831) | (SEQ ID NO: |
| | AQMMQSKSIKKQLRCLWLRVQKEILK | | 841) |
| | (SEQ ID NO: 7) | | |
| Burkholderia | MSEPVEWFKRRRYLHFDLPTSEPAAVAIA | GCTCATCGGATTTGCCGG | TAGAAATG |
| cepacian (Bce) | GNPSIVARHAFYPFIQFTVKSRKVKWDKL | TGAGGAAAGTGACAGGTT | CAGTATCAT |
| | TKRLVTTEKERPVSYASHVDSHIYACYAS | GTCAAGCCACGCGAATCC | GGCAGTAT |
| | ILSQRYEDYLKEVGYQSSVLAFRKLGKSN | GCTCCATGAGCTGAGACG | CATCGTAG |
| | IEFAKDAFDAIKEMGGADVIAADISDFFGS | CAACGCGGACAACACGTA | AGGTCCGT |
| | LNHKILKASWAKILKVNELPNDHYNVFK | GAACAGGACTTCCGTCAT | ATGACGGA |
| | SLTRFTWVDKTNLYKALGISIYNPRGSGA | ACGGACCTCTACGATGAT | AGTCCTGTT |
| | RICTPEIFRSVVRKLGLIQRNAADKGIPQG | ACTGCCATGATACTGCAT | CTACGTGTT |
| | SPISALLSNIYMTDFDQALWTSIASMGGK | TTCTAACATTTAGCGGCC | GTCCGCGTT |
| | YFRYCDDMLIIAPEGKGKEFMRLARELA | AAAGCTCAGTTTTCGGAC | GCGTCTCAG |
| | DLYALPLHPKKTEERFFTSSSGKLLADKP | TGAGTGAGATAATGTGAA | CTCATGGA |
| | LQYLGFVFDGQQVSLRSASLARYSDRMR | GAACATTGTTGTATATCC | GCGGATTC |
| | RGVRLARVTMHARNTLRVERGESPRPLY | GGGGAGGCGGTTATTGGA | GCGTGGCTT |
| | KGKLYRRYSYLGRRNFVSYAIRAANILDE | ATGCCGCCAACCATCGCC | GACAACCT |
| | KAIKRQVKPLWKRLRSQMK (SEQ ID NO: | TCTCCGGTTTGCTAG (SEQ | GT (SEQ ID |
| | 8) | ID NO: 832) | NO: 842) |
| Pseudomonas | MDELPWYKPRKYLHFDRPVGQKAALDY | GCTTGTGGCATTAGGCCA | TAGAGTTG |
| syringae (Psy) | VSSPQKVAAHSFYPFITYTSVILKYRLDND | CGAGGTGGATCACAAGTT | ACGTATAGT |
| | RGVYVKNPKPRPISYSAHLDSHIYAYYCF | GTCAAGCCACCCATGATT | GTCAGTATC |
| | VLSQLYEKELVRRGLHTSVLAFRSIDGKS | ATTTATCAGCGGGACACA | ACGATTAC |
| | NIHFAHDAFCEIKAKGECCVLAFDVEKFF | CATGGCGAACAACAGCAC | ATCTGGAG |
| | DRLDHSRLKAAWAKLLGVGKLPADHFA | GCGGAATCTCTTCATTGA | GTTCTCAAT |
| | VFKSITKSSRVDKVALYDKLGVSIHNPRV | GAACCTCCAGATGTAATC | GAAGAGAT |
| | EDKRLCSAKRFRDDVRGGGLLTTNPESR | GTGATACTGACACTATAC | TCCGCGTGC |
| | GIPQGSPISALLSNIYMLEFDLALISKLSLT | GTCAACTCTAGCAATACG | TGTTGTTCG |
| | ESVFHRYCDDILVICDSEKEDEIYEYVNDE | CGGCCTAGTCTGAAAGGA | CCATGTGTG |
| | IRKLGLNLQEKKTEIRQFAFDAAGKLRSD | TGGATAATGTTTAGAGCA | TCCCGCTGA |
| | KPVHYLGFSFDGQREHIRSSSITRYYKKLR | TTAAGTTGAGCCGGTGGG | TAAATAATC |
| | KRIWVSKKAMDRCNELRAGRGELPRELF | GCGGTTTAGGGATTGCCG | ATGGGTGG |
| | LRTIYRGYSYLGRRNFISYGFRAANIMDS | CCATCCTAGTCCCACCGG | CTTGACAAC |
| | QSIRKQLRSHWKKLNKLLADARSNNQ | TTGGCTTA (SEQ ID NO: | TTGT (SEQ |
| | (SEQ ID NO: 9) | 833) | ID NO: 843) |
| Stutzerimonas | MAEAWYKSRGYLHFDRPVNQATATEIVS | GCTTTGGATGTAGATCCA | TAGGATCG |
| stutzeri (Sst) | SPDAVSRHAFYPFIRYVAQTQKVFFDKGL | AAGGTGCATCACAAGCTG | ACGTATAAT |
| | GKVVKKEPKKRPISYAAHVDSHIYSYYCE | TCAAGGCCCATCGCGGCA | GTCAGCGTC |
| | KLSKAYEDHLGKVPWSSSILAFRSLGKSN | CCACAGGTGAAATAACCC | ACACTTGG |
| | IDFARDAFLDISVRDSSCVIAIDIKGFFENL | AGGGTGACCAGCAGTGCG | AGTACCTAT |
| | NHAHLKKSWQTLLGTSTLPDDHYAVYRS | GCCAGCAGCGCTCGGATA | GAGAATAA |
| | LTKYSFVNRDQVYEVLGLSKNNPKNGRK | CTTATTCTCATAGGTACTC | GCGCTGCTG |
| | RICEPQKFREKVRNGGLIETNKLNKGIPQ | CAAGTGTGACGCTGACAT | GCCGCACT |
| | GSPISAMLSNVYMMGFDEQVHAYVQAC | TATACGTCGATCCTAGCA | GCTGGTCAC |
| | GGSYYRYCDDVLLIVPLAKEAEAKDFVN | ATCGCGGCGAAATCGGAA | CCTGGGTTA |
| | QRVDEIGLEIQEAKTETCKFTRSAKGLRT | CTGGGTGACTATGACCGA | TTTCACCTG |
| | DRPLQYLGFIFDGANIYLRSSSLARYQDR | TTGGATGATGTGAGGAGC | TGGTGCCGC |
| | VNRGIWVAGKCMDKVNAKRISRGQLPRS | ATTTTGGTTTATCCGGCC | GATGGGCC |
| | MFLKKLYKRYSYLGRRNFISYGYRAAKI | AGGCGGCTTATGGGATTG | TTGACAGCT |
| | MDSPSIRKQLKPHWRRLRERISDAQGE | CCGCCATCCTAAGCCTGG | GTATCCGA |
| | (SEQ ID NO: 10) | CCGGTTGGCTAA (SEQ ID | TGT (SEQ ID |
| | | NO: 834) | NO: 844) |

TABLE 2
Strains

| Strain | Description | Genotype | Source |
|---|---|---|---|
| sSL0410 | E. coli | F′ proA + B + lacIq ΔlacZM15/fhuA2 Δ(lac- | New England |
| | NEB Turbo | proAB) glnV galK16 galE15 R(zgb- | BioLabs (C2984H) |
| | | 210::Tn10)TetS endA1 thi-1 Δ(hsdS-mcrB)5 | |
| sSL0810 | E. coli | F- lambda- ilvG- rfb-50 rph-1 | Yale E. coli Genetic |
| | MG1655 | | Stock Center |

TABLE 3
Sequences of DRT2 ncRNAs

| ncRNA | | SEQ |
|---|---|---|
| Description | ncRNA sequence | ID NO |
| WT | GCCCUAAACAAAGGUUUAGGGGUAUUGUACAGGUUGUCAAGCCUCCCACAG | 827 |
| | GUCUUGGUGAAACCAAUCACUGUGACGACGGUAAGCAACACUUGGAUGAUA | |
| | UUCAUAAUUGACUCCACGCUACUGAUUACAUUAUACAGCAUAUCUAACAUU | |
| | UGCGGCGAGGUUCACAAUUUGUAUUUAGGUACUGAUUGUGGAUGAGAAGGU | |
| | UGGAGAAAGACCACUUGGUUAAGCCGGAGGAUGUGUCCUAGAAUUGUCGCU | |
| | AUUCUGUCAUCCUCCGGUUUUGCUAA | |
| scramble_SL6 | GCCCUAAACAAAGGUUUAGGGGUAUUGUACAGGUUGUCAAGCCUCCCACAG | 846 |
| | GUCUUGGUGAAACCAAUCACUGUGACGACGGUAAGCAACACUUGGAUGAUA | |
| | UUCAUAAUUGACUCCACGCUACUGAUUACAUUAUACAGCAUAUCUAACAUU | |
| | UGCGGCGAGUGUGUGUUAUAGUGUUGGGAUACCUAAAUCUUAUGAGAAGGU | |
| | UGGAGAAAGACCACUUGGUUAAGCCGGAGGAUGUGUCCUAGAAUUGUCGCU | |
| | AUUCUGUCAUCCUCCGGUUUUGCUAA | |
| scramble_SL7 | GCCCUAAACAAAGGUUUAGGGGUAUUGUACAGGUUGUCAAGCCUCCCACAG | 847 |
| | GUCUUGGUGAAACCAAUCACUGUGACGACGGUAAGCAACACUUGGAUGAUA | |
| | UUCAUAAUUGACUCCACGCUACUGAUUACAUUAUACAGCAUAUCUAACAUU | |
| | UGCGGCGAGGUUCACAAUUUGUAUUUAGGUACUGAUUGUGGAUGAGAAAGG | |
| | GAACGUAGACUGACUUGGUUAAGCCGGAGGAUGUGUCCUAGAAUUGUCGCU | |
| | AUUCUGUCAUCCUCCGGUUUUGCUAA | |
| scramble_SL8 | GCCCUAAACAAAGGUUUAGGGGUAUUGUACAGGUUGUCAAGCCUCCCACAG | 848 |
| | GUCUUGGUGAAACCAAUCACUGUGACGACGGUAAGCAACACUUGGAUGAUA | |
| | UUCAUAAUUGACUCCACGCUACUGAUUACAUUAUACAGCAUAUCUAACAUU | |
| | UGCGGCGAGGUUCACAAUUUGUAUUUAGGUACUGAUUGUGGAUGAGAAGGU | |
| | UGGAGAAAGACCACUUUAUGGUUGGCGACAUUUUAUCGAGAACGUUAGGAG | |
| | GGCCUUCUUCGGCUCUUCGUACUCUA | |
| 3′ins5 | GCCCUAAACAAAGGUUUAGGGGUAUUGUACAGGUUGUCAAGCCUCCCACAG | 849 |
| | GUCUUGGUGAAACCAAUCACUGUGACGACGGUAAGCAACACUUGGAUGAUA | |
| | UUCAUAAUUGACUCCACGCUACUGAUUACAUUAUACAGCAUAUCUAACAUU | |
| | UGCGGCGAGGUUCACAAUUUGUAUUUAGGUACUGAUUGUGGAUGAGAAGGU | |
| | UGGAGAAAGACCACUUGGUUAAGCCGGAGGAUGUGUCCUAGAAUUGUCGCU | |
| | AUUCUGUCAUCCUCCGGUUUUGCUAAUAUUC | |
| mutate_ACA-2 | GCCCUAAACAAAGGUUUAGGGGUAUUGUACAGGUUGUCAAGCCUCCCACAG | 850 |
| | GUCUUGGUGAAACCAAUCACUGUGACGACGGUAAGCAACACUUGGAUGAUA | |
| | UUCAUAAUUGACUCCACGCUACUGAUUACAUUAUACAGCAUAUCUACGCUU | |
| | UGCGGCGAGGUUCACAAUUUGUAUUUAGGUACUGAUUGUGGAUGAGAAGGU | |
| | UGGAGAAAGACCACUUGGUUAAGCCGGAGGAUGUGUCCUAGAAUUGUCGCU | |
| | AUUCUGUCAUCCUCCGGUUUUGCUAA | |
| rescueSL2_ | GCCCUAAACAAAGGUUUAGGGGUAUUCCGGAGGUUGUCAAGCCUCCCACAG | 851 |
| GGAhomology | GUCUUGGUGAAACCAAUCACUGUGACGACGGUAAGCAACACUUGGAUGAUA | |
| | UUCAUAAUUGACUCCACGCUACUGAUUACAUUAUACAGCAUAUCUAGGAUU | |
| | UGCGGCGAGGUUCACAAUUUGUAUUUAGGUACUGAUUGUGGAUGAGAAGGU | |
| | UGGAGAAAGACCACUUGGUUAAGCCGGAGGAUGUGUCCUAGAAUUGUCGCU | |
| | AUUCUGUCAUCCUCCGGUUUUGCUAA | |
| rand20 | GCCCUAAACAAAGGUUUAGGGGUAUUGUACAGGUUGUCAAGCCUCCCACAG | 852 |
| | GUCUUGGUGAAACCAAUCACUGUGACGUGAAUCUUUACUCCCAAGCGGAUA | |
| | UUCAUAAUUGACUCCACGCUACUGAUUACAUUAUACAGCAUAUCUAACAUU | |
| | UGCGGCGAGGUUCACAAUUUGUAUUUAGGUACUGAUUGUGGAUGAGAAGGU | |
| | UGGAGAAAGACCACUUGGUUAAGCCGGAGGAUGUGUCCUAGAAUUGUCGCU | |
| | AUUCUGUCAUCCUCCGGUUUUGCUAA | |
| rand40 | GCCCUAAACAAAGGUUUAGGGGUAUUGUACAGGUUGUCAAGCCUCCCACAG | 853 |
| | GUCUUGGUGAAACCAAUUGGACUAAUAUGAAUCUUUACUCCCAAGCGAAAA | |
| | CGGGUGAUUGACUCCACGCUACUGAUUACAUUAUACAGCAUAUCUAACAUU | |
| | UGCGGCGAGGUUCACAAUUUGUAUUUAGGUACUGAUUGUGGAUGAGAAGGU | |
| | UGGAGAAAGACCACUUGGUUAAGCCGGAGGAUGUGUCCUAGAAUUGUCGCU | |
| | AUUCUGUCAUCCUCCGGUUUUGCUAA | |
| rand60 | GCCCUAAACAAAGGUUUAGGGGUAUUGUACAGGUUGUCAAGCCUCCCACAG | 854 |
| | GUCUUGGUAGCAUCAGGUGGACUAAUAUGAAUCUUUACUCCCAAGCGAAAA | |
| | CGGGUGCGUGGACUAGCGCUACUGAUUACAUUAUACAGCAUAUCUAACAUU | |
| | UGCGGCGAGGUUCACAAUUUGUAUUUAGGUACUGAUUGUGGAUGAGAAGGU | |
| | UGGAGAAAGACCACUUGGUUAAGCCGGAGGAUGUGUCCUAGAAUUGUCGCU | |
| | AUUCUGUCAUCCUCCGGUUUUGCUAA | |
| rand80 | GCCCUAAACAAAGGUUUAGGGGUAUUGUACAGGUUGUCAAGCCUCCCAUAU | 855 |
| | UUUACGUUAGCAUCAGGUGGACUAAUAUGAAUCUUUACUCCCAAGCGAAAA | |
| | CGGGUGCGUGGACUAGCGAGGAGCAAUACAUUAUACAGCAUAUCUAACAUU | |
| | UGCGGCGAGGUUCACAAUUUGUAUUUAGGUACUGAUUGUGGAUGAGAAGGU | |
| | UGGAGAAAGACCACUUGGUUAAGCCGGAGGAUGUGUCCUAGAAUUGUCGCU | |
| | AUUCUGUCAUCCUCCGGUUUUGCUAA | |
| rand100 | GCCCUAAACAAAGGUUUAGGGGUAUUGUACAGGUUGUCGUCCCCAGGGUAU | 856 |
| | UUUACGUUAGCAUCAGGUGGACUAAUAUGAAUCUUUACUCCCAAGCGAAAA | |
| | CGGGUGCGUGGACUAGCGAGGAGCAAACGAAAAUUCAGCAUAUCUAACAUU | |
| | UGCGGCGAGGUUCACAAUUUGUAUUUAGGUACUGAUUGUGGAUGAGAAGGU | |
| | UGGAGAAAGACCACUUGGUUAAGCCGGAGGAUGUGUCCUAGAAUUGUCGCU | |
| | AUUCUGUCAUCCUCCGGUUUUGCUAA | |
| rand117 | GCCCUAAACAAAGGUUUAGGGGUAUUGUACACGUCGGCGUCCCCAGGGUAU | 857 |
| | UUUACGUUAGCAUCAGGUGGACUAAUAUGAAUCUUUACUCCCAAGCGAAAA | |
| | CGGGUGCGUGGACUAGCGAGGAGCAAACGAAAAUUCUUGGCCUGCUACAUU | |
| | UGCGGCGAGGUUCACAAUUUGUAUUUAGGUACUGAUUGUGGAUGAGAAGGU | |
| | UGGAGAAAGACCACUUGGUUAAGCCGGAGGAUGUGUCCUAGAAUUGUCGCU | |
| | AUUCUGUCAUCCUCCGGUUUUGCUAA | |
| rand120 | GCCCUAAACAAAGGUUUAGGGGUAUUGUUACCGUCGGCGUCCCCAGGGUAU | 858 |
| | UUUACGUUAGCAUCAGGUGGACUAAUAUGAAUCUUUACUCCCAAGCGAAAA | |
| | CGGGUGCGUGGACUAGCGAGGAGCAAACGAAAAUUCUUGGCCUGCUACAUU | |
| | UGCGGCGAGGUUCACAAUUUGUAUUUAGGUACUGAUUGUGGAUGAGAAGGU | |
| | UGGAGAAAGACCACUUGGUUAAGCCGGAGGAUGUGUCCUAGAAUUGUCGCU | |
| | AUUCUGUCAUCCUCCGGUUUUGCUAA | |
| scramble_SL3 | GCCCUAAACAAAGGUUUAGGGGUAUUGUACACAUUAUGCAGCCUGGUUGGA | 859 |
| | UCAAUACCCAAGAGAACCGCGAACCUACCGCUGAUUAUGGGCUGGAUGAUA | |
| | UUCAUAAUUGACUCCACGCUACUGAUUACAUUAUACAGCAUAUCUAACAUU | |
| | UGCGGCGAGGUUCACAAUUUGUAUUUAGGUACUGAUUGUGGAUGAGAAGGU | |
| | UGGAGAAAGACCACUUGGUUAAGCCGGAGGAUGUGUCCUAGAAUUGUCGCU | |
| | AUUCUGUCAUCCUCCGGUUUUGCUAA | |
| scramble_SL4 | GCCCUAAACAAAGGUUUAGGGGUAUUGUACAGGUUGUCAAGCCUCCCACAG | 860 |
| | GUCUUGGUGAAACCAAUCACUGUGACGACGGUAAGCAACACUAGCAUUCUU | |
| | UUGAAUUAGCUACAGACGCUACUGAUUACAUUAUACAGCAUAUCUAACAUU | |
| | UGCGGCGAGGUUCACAAUUUGUAUUUAGGUACUGAUUGUGGAUGAGAAGGU | |
| | UGGAGAAAGACCACUUGGUUAAGCCGGAGGAUGUGUCCUAGAAUUGUCGCU | |
| | AUUCUGUCAUCCUCCGGUUUUGCUAA | |
| comp_U147- | GCCCUAAACAAAGGUUUAGGGGUAUUGUACACCAACAGUUCGGAGGGUGUC | 861 |
| A148_mutated | CAGAACCACUUUGGUUAGUGACACUGCUGCCAUUCGUUGUGAACCUACUAU | |
| | AAGUAUUAACUGAGGUGCGAUGACUAAUGUAAUAUGUCGUAUAGAUACAUU | |
| | UGCGGCGAGGUUCACAAUUUGUAUUUAGGUACUGAUUGUGGAUGAGAAGGU | |
| | UGGAGAAAGACCACUUGGUUAAGCCGGAGGAUGUGUCCUAGAAUUGUCGCU | |
| | AUUCUGUCAUCCUCCGGUUUUGCUAA | |
| comp_U147- | GCCCUAAACAAAGGUUUAGGGGUAUUGUACACCAACAGUUCGGAGGGUGUC | 862 |
| A148_retained | CAGAACCACUUUGGUUAGUGACACUGCUGCCAUUCGUUGUGAACCUACUAU | |
| | AAGUAUUAACUGAGGUGCGAUGACUAAUGUAAUAUGUCGUAUAGUAACAUU | |
| | UGCGGCGAGGUUCACAAUUUGUAUUUAGGUACUGAUUGUGGAUGAGAAGGU | |
| | UGGAGAAAGACCACUUGGUUAAGCCGGAGGAUGUGUCCUAGAAUUGUCGCU | |
| | AUUCUGUCAUCCUCCGGUUUUGCUAA | |
| Kpn > Vch | GCCCUAAACAAAGGUUUAGGGGUAUUGUACAGGUUGUCAAGUCCAAUCGGC | 863 |
| template | ACGCCACCAAGGCAAGCUGAGCAGAUAGACAGCAUGAACUGGAACAGGGUC | |
| | UUCAUAGAUACCUCCACGGUUAUAGUUAUAGCAUAAUCCAUUUCUAACAUU | |
| | UGCGGCGAGGUUCACAAUUUGUAUUUAGGUACUGAUUGUGGAUGAGAAGGU | |
| | UGGAGAAAGACCACUUGGUUAAGCCGGAGGAUGUGUCCUAGAAUUGUCGCU | |
| | AUUCUGUCAUCCUCCGGUUUUGCUAA | |
| ins250_SL4 | GCCCUAAACAAAGGUUUAGGGGUAUUGUACAGGUUGUCAAGCCUCCCACAG | 864 |
| | GUCUUGGUGAAACCAAUCACUGUGACGACGGUAAGCAACACUUGGAUGAUU | |
| | GCUAGAGGAUGGAAUGACCUUAAAUCAGGGACGAUAUUAAACGGAACGUAU | |
| | AUUCAACGCAAUGAAGCCGGAGGAUUGGCGUGGGAAUCGUGCUUCUGUCUA | |
| | AGCAAGUAAGGGUAUGAGGUCGCAACCGUCCCCCAAGCGUACAGGGUGCAC | |
| | UUUGUAACGAUUUGGGAGUCCAGAGACUCGCUGUUUUCGAAAUUUGCCCUC | |
| | AAGCGCGAGUAUUGAACCAGGCUUACGCCCAAGAACGUAGCAAGCAUUCAU | |
| | AAUUGACUCCACGCUACUGAUUACAUUAUACAGCAUAUCUAACAUUUGCGG | |
| | CGAGGUUCACAAUUUGUAUUUAGGUACUGAUUGUGGAUGAGAAGGUUGGAG | |
| | AAAGACCACUUGGUUAAGCCGGAGGAUGUGUCCUAGAAUUGUCGCUAUUCU | |
| | GUCAUCCUCCGGUUUUGCUAA | |
| ins100_SL4 | GCCCUAAACAAAGGUUUAGGGGUAUUGUACAGGUUGUCAAGCCUCCCACAG | 865 |
| | GUCUUGGUGAAACCAAUCACUGUGACGACGGUAAGCAACACUUGGAUGAUU | |
| | GCUAGAGGAUGGAAUGACCUUAAAUCAGGGACGAUAUUAAACGGAACGUCC | |
| | CUCAAGCGCGAGUAUUGAACCAGGCUUACGCCCAAGAACGUAGCAAGCAUU | |
| | CAUAAUUGACUCCACGCUACUGAUUACAUUAUACAGCAUAUCUAACAUUUG | |
| | CGGCGAGGUUCACAAUUUGUAUUUAGGUACUGAUUGUGGAUGAGAAGGUUG | |
| | GAGAAAGACCACUUGGUUAAGCCGGAGGAUGUGUCCUAGAAUUGUCGCUAU | |
| | UCUGUCAUCCUCCGGUUUUGCUAA | |
| sub_miniGFP1 | GCCCUAAACAAAGGUUUAGGGGUAUUGUACAUUAAACGUGGUCUGACCCCA | 866 |
| | CCAGCUGGACACCAAUAAAAUAUUGAAGUCCACCUUUGCCGUCAAAGACCG | |
| | GCUGCAAGUGCAGUAAGUUCCAAAACUUUUUCCCGGAUUUCGUGUAGUUGA | |
| | UAAGCUGAACGGUCGUCGGGCGACGGUCGCGGAUAGCGUCACGAAUUUUUU | |
| | GCACCGUGGCUUGGUCAGUUUCUGGCCCCUGCAAAAAACGCGCGUUGCGUCC | |
| | CAUAAUCUCUUCACGGGAAUACUCCGUCAAUUCCAAGAAACCAUCUGAAGC | |
| | UGAAAUGAUCGGGUAAUCAGGCAGCCAGGGGUCAGUAAUCACGAACGAUUU | |
| | UUCCAUACAUUUGCGGCGAGGUUCACAAUUUGUAUUUAGGUACUGAUUGUG | |
| | GAUGAGAAGGUUGGAGAAAGACCACUUGGUUAAGCCGGAGGAUGUGUCCUA | |
| | GAAUUGUCGCUAUUCUGUCAUCCUCCGGUUUUGCUAA | |
| 3′de15 | GCCCUAAACAAAGGUUUAGGGGUAUUGUACAGGUUGUCAAGCCUCCCACAG | 3436 |
| | GUCUUGGUGAAACCAAUCACUGUGACGACGGUAAGCAACACUUGGAUGAUA | |
| | UUCAUAAUUGACUCCACGCUACUGAUUACAUUAUACAGCAUAUCUAACAUU | |
| | UGCGGCGAGGUUCACAAUUUGUAUUUAGGUACUGAUUGUGGAUGAGAAGGU | |
| | UGGAGAAAGACCACUUGGUUAAGCCGGAGGAUGUGUCCUAGAAUUGUCGCU | |
| | AUUCUGUCAUCCUCCGGUUUU | |
| mutSL2_UGU > | GCCCUAAACAAAGGUUUAGGGGUAUUCCACAGGUUGUCAAGCCUCCCACAG | 3437 |
| UCC | GUCUUGGUGAAACCAAUCACUGUGACGACGGUAAGCAACACUUGGAUGAUA | |
| | UUCAUAAUUGACUCCACGCUACUGAUUACAUUAUACAGCAUAUCUAACAUU | |
| | UGCGGCGAGGUUCACAAUUUGUAUUUAGGUACUGAUUGUGGAUGAGAAGGU | |
| | UGGAGAAAGACCACUUGGUUAAGCCGGAGGAUGUGUCCUAGAAUUGUCGCU | |
| | AUUCUGUCAUCCUCCGGUUUUGCUAA | |
| mutACA_ACA- | GCCCUAAACAAAGGUUUAGGGGUAUUGUGGAGGUUGUCAAGCCUCCCACAG | 3438 |
| 1 > GGA | GUCUUGGUGAAACCAAUCACUGUGACGACGGUAAGCAACACUUGGAUGAUA | |
| | UUCAUAAUUGACUCCACGCUACUGAUUACAUUAUACAGCAUAUCUAACAUU | |
| | UGCGGCGAGGUUCACAAUUUGUAUUUAGGUACUGAUUGUGGAUGAGAAGGU | |
| | UGGAGAAAGACCACUUGGUUAAGCCGGAGGAUGUGUCCUAGAAUUGUCGCU | |
| | AUUCUGUCAUCCUCCGGUUUUGCUAA | |
| mutSL2_mutACA- | GCCCUAAACAAAGGUUUAGGGGUAUUGUACAGGUUGUCAAGCCUCCCACAG | 3439 |
| ACA- | GUCUUGGUGAAACCAAUCACUGUGACGACGGUAAGCAACACUUGGAUGAUA | |
| 2 > GGA | UUCAUAAUUGACUCCACGCUACUGAUUACAUUAUACAGCAUAUCUAGGAUU | |
| | UGCGGCGAGGUUCACAAUUUGUAUUUAGGUACUGAUUGUGGAUGAGAAGGU | |
| | UGGAGAAAGACCACUUGGUUAAGCCGGAGGAUGUGUCCUAGAAUUGUCGCU | |
| | AUUCUGUCAUCCUCCGGUUUUGCUAA | |
| rescueSL2_ | GCCCUAAACAAAGGUUUAGGGGUAUUCCGGAGGUUGUCAAGCCUCCCACAG | 3440 |
| GGAhomology | GUCUUGGUGAAACCAAUCACUGUGACGACGGUAAGCAACACUUGGAUGAUA | |
| | UUCAUAAUUGACUCCACGCUACUGAUUACAUUAUACAGCAUAUCUAGGAUU | |
| | UGCGGCGAGGUUCACAAUUUGUAUUUAGGUACUGAUUGUGGAUGAGAAGGU | |
| | UGGAGAAAGACCACUUGGUUAAGCCGGAGGAUGUGUCCUAGAAUUGUCGCU | |
| | AUUCUGUCAUCCUCCGGUUUUGCUAA | |
| ins100_SL3 | GCCCUAAACAAAGGUUUAGGGGUAUUGUACAGGUUGUCAAGCCUCCCACAG | 3441 |
| | GUCUUGGUUGCUAGAGGAUGGAAUGACCUUAAAUCAGGGACGAUAUUAAAC | |
| | GGAACGUCCCUCAAGCGCGAGUAUUGAACCAGGCUUACGCCCAAGAACGUA | |
| | GCAAGCGAAACCAAUCACUGUGACGACGGUAAGCAACACUUGGAUGAUAUU | |
| | CAUAAUUGACUCCACGCUACUGAUUACAUUAUACAGCAUAUCUAACAUUUG | |
| | CGGCGAGGUUCACAAUUUGUAUUUAGGUACUGAUUGUGGAUGAGAAGGUUG | |
| | GAGAAAGACCACUUGGUUAAGCCGGAGGAUGUGUCCUAGAAUUGUCGCUAU | |
| | UCUGUCAUCCUCCGGUUUUGCUAA | |
| ins100_SL5 | GCCCUAAACAAAGGUUUAGGGGUAUUGUACAGGUUGUCAAGCCUCCCACAG | 3442 |
| | GUCUUGGUGAAACCAAUCACUGUGACGACGGUAAGCAACACUUGGAUGAUA | |
| | UUCAUAAUUGACUCCACGCUACUGAUUACUGCUAGAGGAUGGAAUGACCUU | |
| | AAAUCAGGGACGAUAUUAAACGGAACGUCCCUCAAGCGCGAGUAUUGAACC | |
| | AGGCUUACGCCCAAGAACGUAGCAAGCAUUAUACAGCAUAUCUAACAUUUG | |
| | CGGCGAGGUUCACAAUUUGUAUUUAGGUACUGAUUGUGGAUGAGAAGGUUG | |
| | GAGAAAGACCACUUGGUUAAGCCGGAGGAUGUGUCCUAGAAUUGUCGCUAU | |
| | UCUGUCAUCCUCCGGUUUUGCUAA | |
| ins250_SL3 | GCCCUAAACAAAGGUUUAGGGGUAUUGUACAGGUUGUCAAGCCUCCCACAG | 3443 |
| | GUCUUGGUUGCUAGAGGAUGGAAUGACCUUAAAUCAGGGACGAUAUUAAAC | |
| | GGAACGUAUAUUCAACGCAAUGAAGCCGGAGGAUUGGCGUGGGAAUCGUGC | |
| | UUCUGUCUAAGCAAGUAAGGGUAUGAGGUCGCAACCGUCCCCCAAGCGUAC | |
| | AGGGUGCACUUUGUAACGAUUUGGGAGUCCAGAGACUCGCUGUUUUCGAAA | |
| | UUUGCCCUCAAGCGCGAGUAUUGAACCAGGCUUACGCCCAAGAACGUAGCA | |
| | AGCGAAACCAAUCACUGUGACGACGGUAAGCAACACUUGGAUGAUAUUCAU | |
| | AAUUGACUCCACGCUACUGAUUACAUUAUACAGCAUAUCUAACAUUUGCGG | |
| | CGAGGUUCACAAUUUGUAUUUAGGUACUGAUUGUGGAUGAGAAGGUUGGAG | |
| | AAAGACCACUUGGUUAAGCCGGAGGAUGUGUCCUAGAAUUGUCGCUAUUCU | |
| | GUCAUCCUCCGGUUUUGCUAA | |
| ins250_SL5 | GCCCUAAACAAAGGUUUAGGGGUAUUGUACAGGUUGUCAAGCCUCCCACAG | 3444 |
| | GUCUUGGUGAAACCAAUCACUGUGACGACGGUAAGCAACACUUGGAUGAUA | |
| | UUCAUAAUUGACUCCACGCUACUGAUUACUGCUAGAGGAUGGAAUGACCUU | |
| | AAAUCAGGGACGAUAUUAAACGGAACGUAUAUUCAACGCAAUGAAGCCGGA | |
| | GGAUUGGCGUGGGAAUCGUGCUUCUGUCUAAGCAAGUAAGGGUAUGAGGUC | |
| | GCAACCGUCCCCCAAGCGUACAGGGUGCACUUUGUAACGAUUUGGGAGUCC | |
| | AGAGACUCGCUGUUUUCGAAAUUUGCCCUCAAGCGCGAGUAUUGAACCAGG | |
| | CUUACGCCCAAGAACGUAGCAAGCAUUAUACAGCAUAUCUAACAUUUGCGG | |
| | CGAGGUUCACAAUUUGUAUUUAGGUACUGAUUGUGGAUGAGAAGGUUGGAG | |
| | AAAGACCACUUGGUUAAGCCGGAGGAUGUGUCCUAGAAUUGUCGCUAUUCU | |
| | GUCAUCCUCCGGUUUUGCUAA | |
| de120 | GCCCUAAACAAAGGUUUAGGGGUAUUGUACAGGUUGUCAAGCCUCCCACAG | 3445 |
| | GUCUUGGUGAAACCAAUCACUGUGACGGAUAUUCAUAAUUGACUCCACGCU | |
| | ACUGAUUACAUUAUACAGCAUAUCUAACAUUUGCGGCGAGGUUCACAAUUU | |
| | GUAUUUAGGUACUGAUUGUGGAUGAGAAGGUUGGAGAAAGACCACUUGGUU | |
| | AAGCCGGAGGAUGUGUCCUAGAAUUGUCGCUAUUCUGUCAUCCUCCGGUUU | |
| | UGCUAA | |
| de140 | GCCCUAAACAAAGGUUUAGGGGUAUUGUACAGGUUGUCAAGCCUCCCACAG | 3446 |
| | GUCUUGGUGAAACCAAUAUUGACUCCACGCUACUGAUUACAUUAUACAGCA | |
| | UAUCUAACAUUUGCGGCGAGGUUCACAAUUUGUAUUUAGGUACUGAUUGUG | |
| | GAUGAGAAGGUUGGAGAAAGACCACUUGGUUAAGCCGGAGGAUGUGUCCUA | |
| | GAAUUGUCGCUAUUCUGUCAUCCUCCGGUUUUGCUAA | |
| de160 | GCCCUAAACAAAGGUUUAGGGGUAUUGUACAGGUUGUCAAGCCUCCCACAG | 3447 |
| | GUCUUGGCGCUACUGAUUACAUUAUACAGCAUAUCUAACAUUUGCGGCGAG | |
| | GUUCACAAUUUGUAUUUAGGUACUGAUUGUGGAUGAGAAGGUUGGAGAAAG | |
| | ACCACUUGGUUAAGCCGGAGGAUGUGUCCUAGAAUUGUCGCUAUUCUGUCA | |
| | UCCUCCGGUUUUGCUAA | |
| de180 | GCCCUAAACAAAGGUUUAGGGGUAUUGUACAGGUUGUCAAGCCUCCCAUAC | 3448 |
| | AUUAUACAGCAUAUCUAACAUUUGCGGCGAGGUUCACAAUUUGUAUUUAGG | |
| | UACUGAUUGUGGAUGAGAAGGUUGGAGAAAGACCACUUGGUUAAGCCGGAG | |
| | GAUGUGUCCUAGAAUUGUCGCUAUUCUGUCAUCCUCCGGUUUUGCUAA | |
| del100 | GCCCUAAACAAAGGUUUAGGGGUAUUGUACAGGUUGUCAGCAUAUCUAACA | 3449 |
| | UUUGCGGCGAGGUUCACAAUUUGUAUUUAGGUACUGAUUGUGGAUGAGAAG | |
| | GUUGGAGAAAGACCACUUGGUUAAGCCGGAGGAUGUGUCCUAGAAUUGUCG | |
| | CUAUUCUGUCAUCCUCCGGUUUUGCUAA | |
| SL4_ scrambled | GCCCUAAACAAAGGUUUAGGGGUAUUGUACAGGUUGUCAAGCCUCCCACAG | 3450 |
| | GUCUUGGUGAAACCAAUCACUGUGACGACGGUAAGCAACACUAGCAUUCUU | |
| | UUGAAUUAGCUACAGACGCUACUGAUUACAUUAUACAGCAUAUCUAACAUU | |
| | UGCGGCGAGGUUCACAAUUUGUAUUUAGGUACUGAUUGUGGAUGAGAAGGU | |
| | UGGAGAAAGACCACUUGGUUAAGCCGGAGGAUGUGUCCUAGAAUUGUCGCU | |
| | AUUCUGUCAUCCUCCGGUUUUGCUAA | |
| SL5_scrambled | GCCCUAAACAAAGGUUUAGGGGUAUUGUACAGGUUGUCAAGCCUCCCACAG | 3451 |
| | GUCUUGGUGAAACCAAUCACUGUGACGACGGUAAGCAACACUUGGAUGAUA | |
| | UUCAUAAUUGACUCCACGCUACCUAAUUUUUAGCGAAACAUAUCUAACAUU | |
| | UGCGGCGAGGUUCACAAUUUGUAUUUAGGUACUGAUUGUGGAUGAGAAGGU | |
| | UGGAGAAAGACCACUUGGUUAAGCCGGAGGAUGUGUCCUAGAAUUGUCGCU | |
| | AUUCUGUCAUCCUCCGGUUUUGCUAA | |

TABLE 4
Sequences and description of Plasmids

| | | Plasmid DNA |
|---|---|---|
| Plasmid ID | Plasmid name | SEQ ID NO |
| pSL5201 | pACYC184_EV | 867 |
| pSL4878 | pACYC184_KpnDRT2_WT (pLG010) | 868 |
| pSL6978 | pACYC184_KpnDRT2_YCDD > YCAA | 869 |
| pSL4876 | pACYC184_Retron-Ecol_WT (pLG007) | 870 |
| pSL5173 | pACYC184_3xFLAG-KpnDRT2 | 871 |
| pSL5216 | pACYC184_3xFLAG-KpnDRT2_YCDD > YCAA | 872 |
| pSL5147 | pACYC184_Retron-Ecol_WT_3xFLAG | 873 |
| pSL7260 | pACYC184_3xFLAG-KpnDRT2_ncRNA_SL1_scrambled | 874 |
| pSL7268 | pACYC184_3xFLAG-KpnDRT2_ncRNA_SL2_ACA > CGC | 875 |
| pSL6829 | pACYC184_KpnDRT2_ncRNA_SL2_UGU > UCC | 876 |
| pSL6830 | pACYC184_KpnDRT2_ncRNA_ACA-1 > GGA | 877 |
| pSL6831 | pACYC184_KpnDRT2_ncRNA_ACA-2 > GGA | 878 |
| pSL7265 | pACYC184_3xFLAG-KpnDRT2_ncRNA_SL5_scrambled | 879 |
| pSL7269 | pACYC184_3xFLAG-KpnDRT2_ncRNA_SL6_scrambled | 880 |
| pSL7266 | pACYC184_3xFLAG-KpnDRT2_ncRNA_start_scrambled | 881 |
| pSL6547 | pACYC184_KpnDRT2_ncRNA_A48G | 882 |
| pSL6548 | pACYC184_KpnDRT2_ncRNA_A48C | 883 |
| pSL6549 | pACYC184_KpnDRT2_ncRNA_A48T | 884 |
| pSL7276 | pACYC184_KpnDRT2_ncRNA_G112A | 885 |
| pSL7277 | pACYC184_KpnDRT2_ncRNA_G112T | 886 |
| pSL7278 | pACYC184_KpnDRT2_ncRNA_G112C | 887 |
| pSL6777 | pACYC184_KpnDRT2_ncRNA_59insG | 888 |
| pSL6778 | pACYC184_KpnDRT2_ncRNA_59insAG | 889 |
| pSL6779 | pACYC184_KpnDRT2_ncRNA_59insAAG | 890 |
| pSL6780 | pACYC184_KpnDRT2_ncRNA_59insCAAG | 891 |
| pSL6781 | pACYC184_KpnDRT2_ncRNA_59insGCAAG | 892 |
| pSL6782 | pACYC184_KpnDRT2_ncRNA_59insAGCAAG | 893 |
| pSL6783 | pACYC184_KpnDRT2_ncRNA_59insCAGCAAG | 894 |
| pSL6784 | pACYC184_KpnDRT2_ncRNA_59insGCAGCAAG | 895 |
| pSL6785 | pACYC184_KpnDRT2_ncRNA_59insTGCAGCAAG | 896 |
| pSL6787 | pACYC184_KpnDRT2_ncRNA_101insT | 897 |
| pSL6788 | pACYC184_KpnDRT2_ncRNA_101insTA | 898 |
| pSL6789 | pACYC184_KpnDRT2_ncRNA_101insTAA | 899 |
| pSL6790 | pACYC184_KpnDRT2_ncRNA_101insTAAT | 900 |
| pSL6791 | pACYC184_KpnDRT2_ncRNA_101insTAATA | 901 |
| pSL6792 | pACYC184_KpnDRT2_ncRNA_101insTAATAT | 902 |
| pSL6793 | pACYC184_KpnDRT2_ncRNA_101insTAAGTAT | 903 |
| pSL6794 | pACYC184_KpnDRT2_ncRNA_101insTAAGGTAT | 904 |
| pSL6795 | pACYC184_KpnDRT2_ncRNA_101insTAAGGATAT | 905 |
| pSL6590 | pACYC184_KpnDRT2_ncRNA_131insT | 906 |
| pSL6797 | pACYC184_KpnDRT2_ncRNA_131insTA | 907 |
| pSL6798 | pACYC184_KpnDRT2_ncRNA_131insTAC | 908 |
| pSL6799 | pACYC184_KpnDRT2_ncRNA_131insTTAC | 909 |
| pSL6800 | pACYC184_KpnDRT2_ncRNA_131insATTAC | 910 |
| pSL6801 | pACYC184_KpnDRT2_ncRNA_131insAATTAC | 911 |
| pSL6802 | pACYC184_KpnDRT2_ncRNA_131insAAATTAC | 912 |
| pSL6803 | pACYC184_KpnDRT2_ncRNA_131insGAAATTAC | 913 |
| pSL6804 | pACYC184_KpnDRT2_ncRNA_131insTGAAATTAC | 914 |
| pSL6823 | pACYC184_KpnDRT2_ncRNA_T107C | 915 |
| pSL6824 | pACYC184_KpnDRT2_ncRNA_T107A | 916 |
| pSL6825 | pACYC184_KpnDRT2_ncRNA_T107G | 917 |
| pSL7176 | pSC101_EV | 918 |
| pSL7212 | pSC101_KpnDRT2_Neo_WT_1_rep | 919 |
| pSL7211 | pSC101_KpnDRT2_Neo_WT_2_rep | 920 |
| pSL7195 | pSC101_KpnDRT2_Neo_WT_3_rep | 921 |
| pSL7210 | pSC101_KpnDRT2_Neo_scrambled_1_rep | 922 |
| pSL7209 | pSC101_KpnDRT2_Neo_scrambled_2_rep | 923 |
| pSL7197 | pSC101_KpnDRT2_Neo_scrambled_3_rep | 924 |
| pSL7292 | pSC101_KpnDRT2_Neo_N2insP | 925 |
| pSL7291 | pSC101_KpnDRT2_Neo_F16insP | 926 |
| pSL7293 | pSC101_KpnDRT2_Neo_N32insP | 927 |
| pSL6356 | pACYC184_3xFLAG_BceDRT2_WT | 928 |
| pSL6358 | pACYC184_3xFLAG_SstDRT2_WT | 929 |
| pSL6353 | pACYC184_3xFLAG_VcrDRT2_WT | 930 |
| pSL6348 | pACYC184_3xFLAG_VchDRT2_WT | 931 |
| pSL6352 | pACYC184_3xFLAG_YruDRT2_WT | 932 |
| pSL7325 | pSC101_BceDRT2_Neo_WT_1_rep | 933 |
| pSL7324 | pSC101_SstDRT2_Neo_WT_1_rep | 934 |
| pSL7327 | pSC101_VcrDRT2_Neo_WT_1_rep | 935 |
| pSL7323 | pSC101_VchDRT2_Neo_WT_1_rep | 936 |
| pSL7326 | pSC101_YruDRT2_Neo_WT_1_rep | 937 |
| pSL7332 | pSC101_BceDRT2_Neo_WT_2_rep | 938 |
| pSL7331 | pSC101_SstDRT2_Neo_WT_2_rep | 939 |
| pSL7334 | pSC101_VcrDRT2_Neo_WT_2_rep | 940 |
| pSL7330 | pSC101_VchDRT2_Neo_WT_2_rep | 941 |
| pSL7333 | pSC101_YruDRT2_Neo_WT_2_rep | 942 |
| pSL7339 | pSC101_BceDRT2_Neo_WT_3_rep | 943 |
| pSL7338 | pSC101_SstDRT2_Neo_WT_3_rep | 944 |
| pSL7341 | pSC101_VcrDRT2_Neo_WT_3_rep | 945 |
| pSL7337 | pSC101_VchDRT2_Neo_WT_3_rep | 946 |
| pSL7346 | pSC101_BceDRT2_Neo_WT_4_rep | 947 |
| pSL7345 | pSC101_SstDRT2_Neo_WT_4_rep | 948 |
| pSL7348 | pSC101_VcrDRT2_Neo_WT_4_rep | 949 |
| pSL7344 | pSC101_VchDRT2_Neo_WT_4_rep | 950 |
| pSL7642 | pACYC184_3xFLAG_KpnDRT2_scramble_SL7 | 951 |
| pSL7643 | pACYC184_3xFLAG_KpnDRT2_scramble_SL8 | 952 |
| pSL7640 | pACYC184_3xFLAG_KpnDRT2_3′ins5 | 953 |
| pSL6832 | pACYC184_KpnDRT2_rescueSL2_GGAhomology | 954 |
| pSL7608 | pACYC184_3xFLAG_KpnDRT2_rand20 | 955 |
| pSL7609 | pACYC184_3xFLAG_KpnDRT2_rand40 | 956 |
| pSL7610 | pACYC184_3xFLAG_KpnDRT2_rand60 | 957 |
| pSL7611 | pACYC184_3xFLAG_KpnDRT2_rand80 | 958 |
| pSL7612 | pACYC184_3xFLAG_KpnDRT2_rand100 | 959 |
| pSL7613 | pACYC184_3xFLAG_KpnDRT2_rand117 | 960 |
| pSL7615 | pACYC184_3xFLAG_KpnDRT2_rand120 | 961 |
| pSL7617 | pACYC184_3xFLAG_KpnDRT2_scramble_SL3 | 962 |
| pSL7264 | pACYC184_3xFLAG_KpnDRT2_scramble_SL4 | 963 |
| pSL7618 | pACYC184_3xFLAG_KpnDRT2_comp_U147- | 964 |
| | A148_mutated | |
| pSL7619 | pACYC184_3xFLAG_KpnDRT2_comp_U147- | 965 |
| | A148_retained | |
| pSL6914 | pACYC184_KpnDRT2_Kpn > Vch template | 966 |
| pSL7624 | pACYC184_3xFLAG_KpnDRT2_ins250_SL4 | 967 |
| pSL7621 | pACYC184_3xFLAG_KpnDRT2_ins100_SL4 | 968 |
| pSL7626 | pACYC184_3xFLAG_KpnDRT2_sub_miniGFP1 | 969 |
| pSL7503 | pcDNA3.1_BPNLS_3xFLAG_HCO-KpnDRT2 (pRT) | 970 |
| pSL2268 | pSelection | 971 |
| pSL7516 | U6-Kpn_ncRNA_ribozyme_polyT (pncRNA-1) | 972 |
| pSL7517 | U6-Kpn_ncRNA_polyT (pncRNA-2) | 973 |
| pSL7518 | U6-Kpn_ncRNA_toDRT_polyT (pncRNA-3) | 974 |
| pSL7641 | pACYC184_3xFLAG_KpnDRT2_3′del5 | 3452 |
| pSL7620 | pACYC184_3xFLAG_KpnDRT2_ins100_SL3 | 3453 |
| pSL7622 | pACYC184_3xFLAG_KpnDRT2_ins100_SL5 | 3454 |
| pSL7623 | pACYC184_3xFLAG_KpnDRT2_ins250_SL3 | 3455 |
| pSL7625 | pACYC184_3xFLAG_KpnDRT2_ins250_SL5 | 3456 |
| pSL7635 | pACYC184_3xFLAG_KpnDRT2_del20 | 3457 |
| pSL7636 | pACYC184_3xFLAG_KpnDRT2_del40 | 3458 |
| pSL7637 | pACYC184_3xFLAG_KpnDRT2_del60 | 3459 |
| pSL7638 | pACYC184_3xFLAG_KpnDRT2_del80 | 3460 |
| pSL7639 | pACYC184_3xFLAG_KpnDRT2_del100 | 3461 |
| pSL5328 | pACYC184_J23119_RBS_KpnDRT2_RT | 3462 |
| pSL7872 | pCDF_J23119_KpnDRT2- | 3463 |
| | ncRNA(SL4_scrambled)_KpnDRT2- | |
| | ncRNA(SL5_scrambled) | |
| pSL7873 | pCDF_J23119_KpnDRT2-ncRNA(SL4_scrambled) | 3464 |
| pSL7691 | pCDF_J23119_KpnDRT2-ncRNA(SL5_scrambled) | 3465 |
| pSL7504 | pcDNA3.1_BPNLS_3xFLAG_HCO-BceDRT2 | 3466 |
| pSL7505 | pcDNA3.1_BPNLS_3xFLAG_HCO-SstDRT2 | 3467 |
| pSL7521 | U6-Bce_ncRNA_toDRT_polyT | 3468 |
| pSL7676 | U6-Sst_ncRNA_toDRT_polyT | 3469 |

TABLE 5
DRT2 Homologs

| | | | | Neo coding |
|---|---|---|---|---|
| | | | Neo coding | sequence in |
| | | | sequence in | cDNA repeat |
| | DRT2 | ncRNA | cDNA repeat 1 | 1 + n |
| Organism | SEQ ID NO | SEQ ID NO | SEQ ID NO | SEQ ID NO |
| Vibrio cholerae | 11 | 975 | 1789 | 2594 |
| Vibrio splendidus | 12 | 976 | 1790 | 2595 |
| Vibrio tasmaniensis 1F-267 | 13 | 977 | 1791 | 2596 |
| Stutzerimonas stutzeri XLDN-R | 14 | 978 | 1792 | 2597 |
| Vibrio metoecus | 15 | 979 | 1793 | 2598 |
| Collimonas arenae | 16 | 980 | 1794 | 2599 |
| Vibrio harveyi | 17 | 981 | 1795 | 2600 |
| [Pseudomonas] sp. BICA1-14 | 18 | 982 | 1796 | 2601 |
| Aureimonas sp. Leaf324 | 19 | 983 | 1797 | 2602 |
| Vibrio atlanticus | 20 | 984 | 1798 | 2603 |
| Albimonas donghaensis | 21 | 985 | 1799 | 2604 |
| Pseudomonas resinovorans | 22 | 986 | 1800 | 2605 |
| Stutzerimonas stutzeri | 23 | 987 | 1801 | 2606 |
| Vibrio sp. Vb0932 | 24 | 988 | 1802 | 2607 |
| Inquilinus limosus | 25 | 989 | 1803 | 2608 |
| Bacillus thuringiensis | 26 | 990 | 1804 | 2609 |
| Bacillus anthracis | 27 | 991 | 1805 | 2610 |
| Parvibaculum sp. | 28 | 992 | 1806 | 2611 |
| Vibrio chagasii | 29 | 993 | 1807 | 2612 |
| Photobacterium leiognathi subsp. | 30 | 994 | 1808 | 2613 |
| mandapamensis | | | | |
| Photobacterium leiognathi subsp. | 31 | 995 | 1809 | 2614 |
| mandapamensis | | | | |
| Photobacterium damselae | 32 | 996 | 1810 | 2615 |
| Vibrio pectenicida | 33 | 997 | 1811 | 2616 |
| Klebsiella pneumoniae | 34 | 998 | 1812 | 2617 |
| Pseudoxanthomonas composti | 35 | 999 | 1813 | 2618 |
| Brevundimonas sp. | 36 | 1000 | 1814 | 2619 |
| Vibrio parahaemolyticus | 37 | 1001 | 1815 | 2620 |
| Roseobacter sp. TSBP12 | 38 | 1002 | 1816 | 2621 |
| Pseudovibrio sp. Tun.PSC04-5.I4 | 39 | 1003 | 1817 | 2622 |
| Priestia taiwanensis | 40 | 1004 | 1818 | 2623 |
| Burkholderia cepacia | 41 | 1005 | 1819 | 2624 |
| Dyella sp. ASV21 | 42 | 1006 | 1820 | 2625 |
| Fundidesulfovibrio soli | 43 | 1007 | 1821 | 2626 |
| Vibrio parahaemolyticus | 44 | 1008 | 1822 | 2627 |
| Pseudomonas guariconensis | 45 | 1009 | 1823 | 2628 |
| Vibrio diabolicus | 46 | 1010 | 1824 | 2629 |
| Escherichia coli | 47 | 1011 | 1825 | 2630 |
| Alloalcanivorax xenomutans strain: AGSA2-2 | 48 | 1012 | 1826 | 2631 |
| Lederbergia citrea | 49 | 1013 | 1827 | 2632 |
| Paenalkalicoccus suaedae | 50 | 1014 | 1828 | 2633 |
| Shewanella algae | 51 | 1015 | 1829 | 2634 |
| Shewanella algae | 52 | 1016 | 1830 | 2635 |
| Lysinibacillus capsici | 53 | 1017 | 1831 | 2636 |
| Enterobacter hormaechei | 54 | 1018 | 1832 | 2637 |
| Burkholderia pseudomallei | 55 | 1019 | 1833 | 2638 |
| Vibrio cholerae | 56 | 1020 | 1834 | 2639 |
| Thalassobium sp. | 57 | 1021 | 1835 | 2640 |
| Vibrio parahaemolyticus | 58 | 1022 | 1836 | 2641 |
| Pseudomonas sp. WS 5411 | 59 | 1023 | 1837 | 2642 |
| Vibrio sp. 708 | 60 | 1024 | 1838 | 2643 |
| Pantoea sp. | 61 | 1025 | 1839 | 2644 |
| Pectobacterium versatile | 62 | 1026 | 1840 | 2645 |
| Arsenophonus apicola | 63 | 1027 | 1841 | 2646 |
| Enterobacter asburiae | 64 | 1028 | 1842 | 2647 |
| Vibrio cincinnatiensis | 65 | 1029 | 1843 | 2648 |
| Enterobacter kobei | 66 | 1030 | 1844 | 2649 |
| Providencia alcalifaciens R90-1475 | 67 | 1031 | 1845 | 2650 |
| Burkholderia ubonensis | 68 | 1032 | 1846 | 2651 |
| Pantoea sp. CCBC3-3-1 | 69 | 1033 | 1847 | 2652 |
| Pectobacterium aroidearum | 70 | 1034 | 1848 | 2653 |
| Burkholderia pseudomultivorans | 71 | 1035 | 1849 | 2654 |
| Vibrio sp. SCSIO 43153 | 72 | 1036 | 1850 | 2655 |
| Shewanella sp. SM95 | 73 | 1037 | 1851 | 2656 |
| Enterobacter roggenkampii | 74 | 1038 | 1852 | 2657 |
| Hafnia paralvei | 75 | 1039 | 1853 | 2658 |
| Rhizobium paranaense | 76 | 1040 | 1854 | 2659 |
| Undibacterium seohonense | 77 | 1041 | 1855 | 2660 |
| Thalassospira xiamenensis | 78 | 1042 | 1856 | 2661 |
| Neisseria elongata subsp. glycolytica | 79 | 1043 | 1857 | 2662 |
| ATCC 29315 | | | | |
| Cupriavidus taiwanensis | 80 | 1044 | 1858 | 2663 |
| Xanthomonas campestris pv. | 81 | 1045 | 1859 | 2664 |
| zingibericola | | | | |
| Stenotrophomonas maltophilia | 82 | 1046 | 1860 | 2665 |
| Xanthomonadales bacterium 14-68-21 | 83 | 1047 | 1861 | 2666 |
| Rhizobium sp. CCGE 510 | 84 | 1048 | 1862 | 2667 |
| Pseudomonas aeruginosa | 85 | 1049 | 1863 | 2668 |
| Massilia brevitalea | 86 | 1050 | 1864 | 2669 |
| Pelagibacterium sp. SCN 63-126 | 87 | 1051 | 1865 | 2670 |
| Litoribrevibacter albus | 88 | 1052 | 1866 | 2671 |
| Alteromonas macleodii | 89 | 1053 | 1867 | 2672 |
| Methyloversatilis discipulorum | 90 | 1054 | 1868 | 2673 |
| Conchiformibius kuhniae DSM 17694 | 91 | 1055 | 1869 | 2674 |
| Conchiformibius kuhniae | 92 | 1056 | 1870 | 2675 |
| Agrobacterium sp. MAFF310724 | 93 | 1057 | 1871 | 2676 |
| Agrobacterium tumefaciens | 94 | 1058 | 1872 | 2677 |
| Brucella endophytica | 95 | 1059 | 1873 | 2678 |
| Rhizobium lemnae | 96 | 1060 | 1874 | 2679 |
| Allorhizobium taibaishanense | 97 | 1061 | 1875 | 2680 |
| Rhizobium sp. SSM4.3 | 98 | 1062 | 1876 | 2681 |
| Janthinobacterium sp. HH107 | 99 | 1063 | 1877 | 2682 |
| Pseudomonas sp. S3E12 | 100 | 1064 | 1878 | 2683 |
| Bacillus alkalisoli | 101 | 1065 | 1879 | 2684 |
| Bacillus cereus biovar anthracis str. CI | 102 | 1066 | 1880 | 2685 |
| Photobacterium leiognathi | 103 | 1067 | 1881 | 2686 |
| Photobacterium leiognathi subsp. | 104 | 1068 | 1882 | 2687 |
| mandapamensis | | | | |
| Photobacterium leiognathi subsp. | 105 | 1069 | 1883 | 2688 |
| mandapamensis | | | | |
| Zoogloea oryzae | 106 | 1070 | 1884 | 2689 |
| Janthinobacterium sp. FT14W | 107 | 1071 | 1885 | 2690 |
| Pandoraea iniqua | 108 | 1072 | 1886 | 2691 |
| Moritella sp. | 109 | 1073 | 1887 | 2692 |
| Moritella dasanensis ArB 0140 | 110 | 1074 | 1888 | 2693 |
| Lysinibacillus sp. Al | 111 | 1075 | 1889 | 2694 |
| Aeromonas hydrophila | 112 | 1076 | 1890 | 2695 |
| Aeromonas aquatica | 113 | 1077 | 1891 | 2696 |
| Aeromonas bestiarum | 114 | 1078 | 1892 | 2697 |
| Aeromonas caviae | 115 | 1079 | 1893 | 2698 |
| Rhizobium leguminosarum | 116 | 1080 | 1894 | 2699 |
| Rhizobium sp. SEMIA4064 | 117 | 1081 | 1895 | 2700 |
| Brucella tritici | 118 | 1082 | 1896 | 2701 |
| Rhizobium lentis | 119 | 1083 | 1897 | 2702 |
| Pseudomonas sp. GD03721 | 120 | 1084 | 1898 | 2703 |
| Pseudomonas sp. | 121 | 1085 | 1899 | 2704 |
| Pseudomonas sp. | 122 | 1086 | 1900 | 2705 |
| Pseudomonas corrugata | 123 | 1087 | 1901 | 2706 |
| Pseudomonas sp. GD03985 | 124 | 1088 | 1902 | 2707 |
| Stutzerimonas stutzeri | 125 | 1089 | 1903 | 2708 |
| Stutzerimonas stutzeri A1501 | 126 | 1090 | 1904 | 2709 |
| Stutzerimonas nitrititolerans | 127 | 1091 | 1905 | 2710 |
| Pseudomonas migulae | 128 | 1092 | 1906 | 2711 |
| Pseudomonas campi | 129 | 1093 | 1907 | 2712 |
| Paracoccaceae bacterium | 130 | 1094 | 1908 | 2713 |
| Brucella intermedia | 131 | 1095 | 1909 | 2714 |
| Diaphorobacter sp. | 132 | 1096 | 1910 | 2715 |
| Rhodobacter capsulatus | 133 | 1097 | 1911 | 2716 |
| Rhodobacteraceae bacterium S2214 | 134 | 1098 | 1912 | 2717 |
| Janthinobacterium sp. 67 | 135 | 1099 | 1913 | 2718 |
| Aeromonas dhakensis | 136 | 1100 | 1914 | 2719 |
| Duganella sacchari | 137 | 1101 | 1915 | 2720 |
| Halomonas maura | 138 | 1102 | 1916 | 2721 |
| Desulfurivibrio alkaliphilus AHT 2 | 139 | 1103 | 1917 | 2722 |
| Vibrio nereis | 140 | 1104 | 1918 | 2723 |
| Azospirillum brasilense | 141 | 1105 | 1919 | 2724 |
| Burkholderia sp. B21-005 | 142 | 1106 | 1920 | 2725 |
| Burkholderia cepacia | 143 | 1107 | 1921 | 2726 |
| Paraglaciecola sp. T6c | 144 | 1108 | 1922 | 2727 |
| Burkholderia sp. 9120 | 145 | 1109 | 1923 | 2728 |
| Idiomarina ramblicola | 146 | 1110 | 1924 | 2729 |
| Paraburkholderia fungorum | 147 | 1111 | 1925 | 2730 |
| Pseudomonas savastanoi | 148 | 1112 | 1926 | 2731 |
| Pseudomonas amygdali pv. loropetali | 149 | 1113 | 1927 | 2732 |
| Photobacterium leiognathi subsp. | 150 | 1114 | 1928 | 2733 |
| mandapamensis | | | | |
| Neisseria subflava | 151 | 1115 | 1929 | 2734 |
| Sulfitobacter sabulilitoris | 152 | 1116 | 1930 | 2735 |
| Brevundimonas huaxiensis | 153 | 1117 | 1931 | 2736 |
| Vibrio toranzoniae | 154 | 1118 | 1932 | 2737 |
| Rhizobium glycinendophyticum | 155 | 1119 | 1933 | 2738 |
| Rhizobium ruizarguesonis | 156 | 1120 | 1934 | 2739 |
| Sinorhizobium saheli | 157 | 1121 | 1935 | 2740 |
| Novacetimonas cocois | 158 | 1122 | 1936 | 2741 |
| Flavonifractor sp. An100 | 159 | 1123 | 1937 | 2742 |
| Ignatzschineria cameli | 160 | 1124 | 1938 | 2743 |
| Ignatzschineria indica | 161 | 1125 | 1939 | 2744 |
| Bartonella sp. M0283 | 162 | 1126 | 1940 | 2745 |
| Neisseria dumasiana | 163 | 1127 | 1941 | 2746 |
| Spongiibacter pelagi | 164 | 1128 | 1942 | 2747 |
| uncultured Zhongshania sp. | 165 | 1129 | 1943 | 2748 |
| Hansschlegelia plantiphila | 166 | 1130 | 1944 | 2749 |
| Alcanivorax sp. | 167 | 1131 | 1945 | 2750 |
| Rheinheimera sp. | 168 | 1132 | 1946 | 2751 |
| Sphingomonas pollutisoli | 169 | 1133 | 1947 | 2752 |
| Yersinia ruckeri | 170 | 1134 | 1948 | 2753 |
| Yersinia pseudotuberculosis | 171 | 1135 | 1949 | 2754 |
| Yersinia aleksiciae | 172 | 1136 | 1950 | 2755 |
| Bacillus inaquosorum | 173 | 1137 | 1951 | 2756 |
| Klebsiella sp. ZYC-1 | 174 | 1138 | 1952 | 2757 |
| Aeromonas media | 175 | 1139 | 1953 | 2758 |
| Mitsuokella multacida | 176 | 1140 | 1954 | 2759 |
| Mitsuokella multacida | 177 | 1141 | 1955 | 2760 |
| Yersinia ruckeri | 178 | 1142 | 1956 | 2761 |
| Pseudomonas sp. BLCC-B13 | 179 | 1143 | 1957 | 2762 |
| Pseudomonas sp. Gutcm_11s | 180 | 1144 | 1958 | 2763 |
| Shewanella sp. SM68 | 181 | 1145 | 1959 | 2764 |
| Shewanella baltica OS183 | 182 | 1146 | 1960 | 2765 |
| Pseudomonas mendocina DLHK | 183 | 1147 | 1961 | 2766 |
| Bacillus velezensis | 184 | 1148 | 1962 | 2767 |
| Lysinibacillus capsici | 185 | 1149 | 1963 | 2768 |
| Pseudomonas aeruginosa DQ8 | 186 | 1150 | 1964 | 2769 |
| Pseudomonas sp. BAY1663 | 187 | 1151 | 1965 | 2770 |
| Bacillus mojavensis | 188 | 1152 | 1966 | 2771 |
| Alkalihalobacillus pseudalcaliphilus | 189 | 1153 | 1967 | 2772 |
| Lysinibacillus sp. LK3 | 190 | 1154 | 1968 | 2773 |
| Stutzerimonas stutzeri | 191 | 1155 | 1969 | 2774 |
| Bacillus gobiensis | 192 | 1156 | 1970 | 2775 |
| Agarivorans gilvus | 193 | 1157 | 1971 | 2776 |
| Legionella quinlivanii DSM 21216 | 194 | 1158 | 1972 | 2777 |
| Marinomonas sp. SBI8L | 195 | 1159 | 1973 | 2778 |
| Clostridiales bacterium KLE1615 | 196 | 1160 | 1974 | 2779 |
| Devosia elaeis | 197 | 1161 | 1975 | 2780 |
| Aliivibrio fischeri | 198 | 1162 | 1976 | 2781 |
| Lysinibacillus sp. AR18-8 | 199 | 1163 | 1977 | 2782 |
| Sutcliffiella halmapala | 200 | 1164 | 1978 | 2783 |
| Paracoccus sediminis | 201 | 1165 | 1979 | 2784 |
| Pseudomonas sp. FEMGT703P | 202 | 1166 | 1980 | 2785 |
| Minwuia thermotolerans | 203 | 1167 | 1981 | 2786 |
| Vibrio splendidus | 204 | 1168 | 1982 | 2787 |
| Vibrio splendidus | 205 | 1169 | 1983 | 2788 |
| Stutzerimonas kunmingensis | 206 | 1170 | 1984 | 2789 |
| Vibrio splendidus | 207 | 1171 | 1985 | 2790 |
| Shewanella algae | 208 | 1172 | 1986 | 2791 |
| Ruegeria sp. A3M17 | 209 | 1173 | 1987 | 2792 |
| Blautia sp. TF12-12AT | 210 | 1174 | 1988 | 2793 |
| Bacillus velezensis | 211 | 1175 | 1989 | 2794 |
| Bacillus velezensis | 212 | 1176 | 1990 | 2795 |
| Aliivibrio sp. EL58 | 213 | 1177 | 1991 | 2796 |
| Vibrio crassostreae | 214 | 1178 | 1992 | 2797 |
| Stutzerimonas stutzeri | 215 | 1179 | 1993 | 2798 |
| Colwellia sp. Arc7-635 | 216 | 1180 | 1994 | 2799 |
| Oxalobacteraceae bacterium | 217 | 1181 | 1995 | 2800 |
| Pseudoalteromonas sp. S4741 | 218 | 1182 | ATGT | 2801 |
| Pseudoalteromonas aurantia | 219 | 1183 | 1996 | 2802 |
| Cytobacillus solani | 220 | 1184 | 1997 | 2803 |
| Bradyrhizobium daqingense | 221 | 1185 | 1998 | 2804 |
| Pseudomonas profundi | 222 | 1186 | 1999 | 2805 |
| Pseudooceanicola albus | 223 | 1187 | 2000 | 2806 |
| Pseudomonas nitroreducens | 224 | 1188 | 2001 | 2807 |
| Shewanella sp. ISTPL2 | 225 | 1189 | 2002 | 2808 |
| Pseudomonas ceruminis | 226 | 1190 | 2003 | 2809 |
| Pseudoalteromonas sp. MT33b | 227 | 1191 | ATGT | 2810 |
| Pseudomonas vanderleydeniana | 228 | 1192 | 2004 | 2811 |
| Pseudomonas taiwanensis | 229 | 1193 | 2005 | 2812 |
| Pseudoalteromonas sp. APC 3215 | 230 | 1194 | ATGT | 2813 |
| Zobellella iuensis | 231 | 1195 | 2006 | 2814 |
| Parahaliea mediterranea | 232 | 1196 | 2007 | 2815 |
| Pseudomonas sp. PNP | 233 | 1197 | 2008 | 2816 |
| Bacillus subtilis | 234 | 1198 | 2009 | 2817 |
| Oceanimonas baumannii | 235 | 1199 | 2010 | 2818 |
| Vibrio kanaloae | 236 | 1200 | 2011 | 2819 |
| Klebsiella aerogenes | 237 | 1201 | 2012 | 2820 |
| Aeromonas jandaei | 238 | 1202 | 2013 | 2821 |
| Enterobacter hormaechei | 239 | 1203 | 2014 | 2822 |
| Pantoea ananatis | 240 | 1204 | 2015 | 2823 |
| Klebsiella pneumoniae | 241 | 1205 | 2016 | 2824 |
| Collimonas humicola | 242 | 1206 | 2017 | 2825 |
| Rheinheimera oceanensis | 243 | 1207 | 2018 | 2826 |
| Polymorphobacter sp. PAMC 29334 | 244 | 1208 | 2019 | 2827 |
| Bacillus atrophaeus | 245 | 1209 | 2020 | 2828 |
| Phycisphaeraceae bacterium | 246 | 1210 | 2021 | 2829 |
| Enterobacter roggenkampii | 247 | 1211 | 2022 | 2830 |
| Klebsiella michiganensis E718 | 248 | 1212 | 2023 | 2831 |
| Paraburkholderia tagetis | 249 | 1213 | 2024 | 2832 |
| Vibrio furnissii | 250 | 1214 | 2025 | 2833 |
| Vibrio parahaemolyticus | 251 | 1215 | 2026 | 2834 |
| Ruminococcus sp. AF17-11 | 252 | 1216 | 2027 | 2835 |
| Klebsiella pneumoniae | 253 | 1217 | 2028 | 2836 |
| Cupriavidus basilensis OR16 | 254 | 1218 | 2029 | 2837 |
| Acinetobacter baumannii | 255 | 1219 | 2030 | 2838 |
| Pseudomonas syringae pv. actinidiae | 256 | 1220 | 2031 | 2839 |
| ICMP 19072 | | | | |
| Pectobacterium brasiliense | 257 | 1221 | 2032 | 2840 |
| Photobacterium leiognathi | 258 | 1222 | 2033 | 2841 |
| Flavonifractor plautii | 259 | 1223 | 2034 | 2842 |
| Allomuricauda eckloniae | 260 | 1224 | 2035 | 2843 |
| Duganella sp. CF517 | 261 | 1225 | 2036 | 2844 |
| Azospirillum brasilense | 262 | 1226 | 2037 | 2845 |
| Vibrio sp. | 263 | 1227 | 2038 | 2846 |
| Aeromonas veronii | 264 | 1228 | 2039 | 2847 |
| Acinetobacter pittii | 265 | 1229 | 2040 | 2848 |
| Vibrio splendidus | 266 | 1230 | 2041 | 2849 |
| Tsuneonella flava | 267 | 1231 | 2042 | 2850 |
| Photobacterium phosphoreum | 268 | 1232 | 2043 | 2851 |
| Photobacterium phosphoreum | 269 | 1233 | 2044 | 2852 |
| Photobacterium kishitanii | 270 | 1234 | 2045 | 2853 |
| Photobacterium sp. GB-50 | 271 | 1235 | 2046 | 2854 |
| Acinetobacter haemolyticus | 272 | 1236 | 2047 | 2855 |
| Paraburkholderia sp. BL6669N2 | 273 | 1237 | 2048 | 2856 |
| Chromatocurvus halotolerans | 274 | 1238 | 2049 | 2857 |
| Altericroceibacterium spongiae | 275 | 1239 | 2050 | 2858 |
| Paraburkholderia sp. RAU2J | 276 | 1240 | 2051 | 2859 |
| Burkholderia stabilis | 277 | 1241 | 2052 | 2860 |
| Azospirillum brasilense | 278 | 1242 | 2053 | 2861 |
| Nitrincola iocasae | 279 | 1243 | 2054 | 2862 |
| Klebsiella michiganensis | 280 | 1244 | 2055 | 2863 |
| Escherichia coli | 281 | 1245 | 2056 | 2864 |
| Marinobacter antarcticus | 282 | 1246 | 2057 | 2865 |
| Rugamonas fusca | 283 | 1247 | 2058 | 2866 |
| Aeromonas caviae | 284 | 1248 | 2059 | 2867 |
| Acinetobacter pittii | 285 | 1249 | 2060 | 2868 |
| Aeromonas dhakensis | 286 | 1250 | 2061 | 2869 |
| Oceanobacillus sp. J11TS1 | 287 | 1251 | 2062 | 2870 |
| Burkholderia sp. CpTa8-5 | 288 | 1252 | 2063 | 2871 |
| Aeromonas dhakensis | 289 | 1253 | 2064 | 2872 |
| Sphingomonas ursincola | 290 | 1254 | 2065 | 2873 |
| Paraburkholderia caribensis | 291 | 1255 | 2066 | 2874 |
| Shewanella algae | 292 | 1256 | 2067 | 2875 |
| Rhizobium anhuiense bv. trifolii | 293 | 1257 | 2068 | 2876 |
| Gluconobacter morbifer G707 | 294 | 1258 | 2069 | 2877 |
| Cronobacter sakazakii | 295 | 1259 | 2070 | 2878 |
| Lactobacillus intestinalis | 296 | 1260 | 2071 | 2879 |
| Vibrio cyclitrophicus FF160 | 297 | 1261 | 2072 | 2880 |
| Vibrio fluvialis | 298 | 1262 | 2073 | 2881 |
| Enterobacter hormaechei | 299 | 1263 | 2074 | 2882 |
| Vibrio sp. JCM 18905 | 300 | 1264 | 2075 | 2883 |
| Chrysiogenes arsenatis DSM 11915 | 301 | 1265 | 2076 | 2884 |
| Pseudomonas rhodesiae | 302 | 1266 | 2077 | 2885 |
| Photobacterium angustum | 303 | 1267 | 2078 | 2886 |
| Yersinia similis | 304 | 1268 | 2079 | 2887 |
| Vibrio sp. MEBiC08052 | 305 | 1269 | 2080 | 2888 |
| Photobacterium aquimaris | 306 | 1270 | 2081 | 2889 |
| Vibrio natriegens | 307 | 1271 | 2082 | 2890 |
| Ignavibacteria bacterium RBG_16_35_7 | 308 | 1272 | 2083 | 2891 |
| Bradyrhizobium sp. NFR13 | 309 | 1273 | 2084 | 2892 |
| Cellvibrio sp. PSBB023 | 310 | 1274 | 2085 | 2893 |
| Verrucomicrobia bacterium ADurb.Bin118 | 311 | 1275 | 2086 | 2894 |
| Burkholderia ubonensis | 312 | 1276 | 2087 | 2895 |
| Serratia marcescens | 313 | 1277 | 2088 | 2896 |
| Psychromonas sp. Urea-02u-13 | 314 | 1278 | 2089 | 2897 |
| Vibrio cyclitrophicus | 315 | 1279 | 2090 | 2898 |
| Lysinibacillus sphaericus | 316 | 1280 | 2091 | 2899 |
| Cronobacter sakazakii | 317 | 1281 | 2092 | 2900 |
| Photobacterium angustum | 318 | 1282 | 2093 | 2901 |
| Sphingomonas sp. UV9 | 319 | 1283 | 2094 | 2902 |
| Mesorhizobium composti | 320 | 1284 | 2095 | 2903 |
| Vibrio genomosp. F6 | 321 | 1285 | 2096 | 2904 |
| Rheinheimera tangshanensis | 322 | 1286 | 2097 | 2905 |
| Leclercia adecarboxylata | 323 | 1287 | 2098 | 2906 |
| Devosia sp. Leaf420 | 324 | 1288 | 2099 | 2907 |
| Duganella qianjiadongensis | 325 | 1289 | 2100 | 2908 |
| Caenispirillum salinarum AK4 | 326 | 1290 | 2101 | 2909 |
| Nitrogeniibacter mangrovi | 327 | 1291 | 2102 | 2910 |
| Pantoea multigeneris | 328 | 1292 | 2103 | 2911 |
| Rhizobium leguminosarum bv. viciae | 329 | 1293 | 2104 | 2912 |
| Burkholderia cenocepacia | 330 | 1294 | 2105 | 2913 |
| Klebsiella grimontii | 331 | 1295 | 2106 | 2914 |
| Gluconobacter sp. R75690 | 332 | 1296 | 2107 | 2915 |
| Aeromonas veronii | 333 | 1297 | 2108 | 2916 |
| Pantoea sp. SM3640 | 334 | 1298 | 2109 | 2917 |
| Stutzerimonas stutzeri | 335 | 1299 | 2110 | 2918 |
| Pantoea ananatis LMG 5342 | 336 | 1300 | 2111 | 2919 |
| Sulfitobacter sp. M74 | 337 | 1301 | 2112 | 2920 |
| Acinetobacter baylyi TG19579 | 338 | 1302 | 2113 | 2921 |
| Hyphomonadaceae bacterium UKL13-1 | 339 | 1303 | 2114 | 2922 |
| Acinetobacter soli | 340 | 1304 | 2115 | 2923 |
| Sphaerotilus montanus | 341 | 1305 | 2116 | 2924 |
| Proteus sp. G2669 | 342 | 1306 | 2117 | 2925 |
| Morganella morganii | 343 | 1307 | 2118 | 2926 |
| Pseudomonas otitidis | 344 | 1308 | 2119 | 2927 |
| Actinobacillus pleuropneumoniae | 345 | 1309 | 2120 | 2928 |
| Pseudidiomarina donghaiensis | 346 | 1310 | 2121 | 2929 |
| Idiomarina loihiensis | 347 | 1311 | 2122 | 2930 |
| Rhizobium leguminosarum | 348 | 1312 | 2123 | 2931 |
| Albimonas pacifica | 349 | 1313 | 2124 | 2932 |
| Pseudomonas otitidis | 350 | 1314 | 2125 | 2933 |
| Mannheimia haemolytica D171 | 351 | 1315 | 2126 | 2934 |
| Mannheimia haemolytica | 352 | 1316 | 2127 | 2935 |
| Mannheimia haemolytica | 353 | 1317 | 2128 | 2936 |
| Pectobacterium colocasium | 354 | 1318 | 2129 | 2937 |
| Pectobacterium sp. PL152 | 355 | 1319 | 2130 | 2938 |
| Bacillus cereus | 356 | 1320 | 2131 | 2939 |
| Neisseria weaveri | 357 | 1321 | 2132 | 2940 |
| Aeromonas sp. QDB63 | 358 | 1322 | 2133 | 2941 |
| Aeromonas hydrophila | 359 | 1323 | 2134 | 2942 |
| Aeromonas caviae | 360 | 1324 | 2135 | 2943 |
| Neisseria sp. HMSC70E02 | 361 | 1325 | 2136 | 2944 |
| Agrobacterium larrymoorei | 362 | 1326 | 2137 | 2945 |
| Paracoccus sediminicola | 363 | 1327 | 2138 | 2946 |
| Pseudooceanicola sp. HF7 | 364 | 1328 | 2139 | 2947 |
| Paracoccus alkenifer | 365 | 1329 | 2140 | 2948 |
| Paracoccus pantotrophus J46 | 366 | 1330 | 2141 | 2949 |
| Teredinibacter turnerae T8402 | 367 | 1331 | 2142 | 2950 |
| Eubacterium sp. | 368 | 1332 | 2143 | 2951 |
| 1001713B170207 170306 E7 | | | | |
| Lichenibacterium sp. 6Y81 | 369 | 1333 | 2144 | 2952 |
| Alishewanella jeotgali KCTC 22429 | 370 | 1334 | 2145 | 2953 |
| Vibrio paucivorans | 371 | 1335 | 2146 | 2954 |
| Acinetobacter ursingii | 372 | 1336 | 2147 | 2955 |
| Acinetobacter sp. WCHA39 | 373 | 1337 | 2148 | 2956 |
| Pantoea sp. PSNIH1 | 374 | 1338 | 2149 | 2957 |
| Aeromonas hydrophila | 375 | 1339 | 2150 | 2958 |
| Verrucomicrobiota bacterium | 376 | 1340 | 2151 | 2959 |
| Wohlfahrtiimonas chitiniclastica | 377 | 1341 | 2152 | 2960 |
| Photobacterium damselae subsp. damselae | 378 | 1342 | 2153 | 2961 |
| Exiguobacterium sp. ERU653 | 379 | 1343 | 2154 | 2962 |
| Rhizobium azibense | 380 | 1344 | 2155 | 2963 |
| Salinicoccus cyprini | 381 | 1345 | 2156 | 2964 |
| Psychrobacillus soli | 382 | 1346 | 2157 | 2965 |
| Chromobacterium amazonense | 383 | 1347 | 2158 | 2966 |
| Agrobacterium tumefaciens | 384 | 1348 | 2159 | 2967 |
| Agrobacterium tumefaciens | 385 | 1349 | 2160 | 2968 |
| Agrobacterium tumefaciens | 386 | 1350 | 2161 | 2969 |
| Agrobacterium sp. YIC 4121 | 387 | 1351 | 2162 | 2970 |
| Lysinibacillus sphaericus | 388 | 1352 | 2163 | 2971 |
| Photorhabdus namnaonensis | 389 | 1353 | 2164 | 2972 |
| Providencia rustigianii | 390 | 1354 | 2165 | 2973 |
| Chitinimonas sp. BJYL2 | 391 | 1355 | 2166 | 2974 |
| Plesiomonas shigelloides | 392 | 1356 | 2167 | 2975 |
| Acinetobacter modestus | 393 | 1357 | 2168 | 2976 |
| Acinetobacter modestus | 394 | 1358 | 2169 | 2977 |
| Larkinella punicea | 395 | 1359 | 2170 | 2978 |
| Aeromonas hydrophila | 396 | 1360 | 2171 | 2979 |
| Alphaproteobacteria bacterium HGW- | 397 | 1361 | 2172 | 2980 |
| Alphaproteobacteria-2 | | | | |
| Telluria aromaticivorans | 398 | 1362 | 2173 | 2981 |
| Neisseria zalophi | 399 | 1363 | 2174 | 2982 |
| Bacillus subtilis subsp. subtilis | 400 | 1364 | 2175 | 2983 |
| Acinetobacter johnsonii | 401 | 1365 | 2176 | 2984 |
| Vibrio paracholerae HE-16 | 402 | 1366 | 2177 | 2985 |
| Vibrio paracholerae HE-16 | 403 | 1367 | 2178 | 2986 |
| Vibrio tasmaniensis 1F-267 | 404 | 1368 | 2179 | 2987 |
| Vibrio metoecus | 405 | 1369 | 2180 | 2988 |
| Vibrio harveyi NBRC 15634 = ATCC | 406 | 1370 | 2181 | 2989 |
| 14126 = KCTC 12724 | | | | |
| [Pseudomonas] sp. BICA1-14 | 407 | 1371 | 2182 | 2990 |
| Vibrio atlanticus | 408 | 1372 | 2183 | 2991 |
| Pseudomonas resinovorans | 409 | 1373 | 2184 | 2992 |
| Stutzerimonas stutzeri | 410 | 1374 | 2185 | 2993 |
| Vibrio alginolyticus | 411 | 1375 | 2186 | 2994 |
| Vibrio sp. T3Y01 | 412 | 1376 | 2187 | 2995 |
| Vibrio splendidus | 413 | 1377 | 2188 | 2996 |
| Vibrio splendidus | 414 | 1378 | 2189 | 2997 |
| Photobacterium damselae | 415 | 1379 | 2190 | 2998 |
| Vibrio pectenicida | 416 | 1380 | 2191 | 2999 |
| Klebsiella pneumoniae | 417 | 1381 | 2192 | 3000 |
| Vibrio parahaemolyticus | 418 | 1382 | 2193 | 3001 |
| Vibrio parahaemolyticus | 419 | 1383 | 2194 | 3002 |
| Vibrio parahaemolyticus | 420 | 1384 | 2195 | 3003 |
| Vibrio cholerae | 421 | 1385 | 2196 | 3004 |
| Vibrio cholerae | 422 | 1386 | 2197 | 3005 |
| Vibrio parahaemolyticus | 423 | 1387 | 2198 | 3006 |
| Vibrio cholerae | 424 | 1388 | 2199 | 3007 |
| Burkholderia cepacia | 425 | 1389 | 2200 | 3008 |
| Klebsiella pneumoniae subsp. | 426 | 1390 | 2201 | 3009 |
| pneumoniae 1084 | | | | |
| Klebsiella pneumoniae | 427 | 1391 | 2202 | 3010 |
| Klebsiella pneumoniae | 428 | 1392 | 2203 | 3011 |
| Vibrio alginolyticus | 429 | 1393 | 2204 | 3012 |
| Vibrio parahaemolyticus | 430 | 1394 | 2205 | 3013 |
| Vibrio chagasii | 431 | 1395 | 2206 | 3014 |
| Vibrio chagasii | 432 | 1396 | 2207 | 3015 |
| Pseudomonas putida | 433 | 1397 | 2208 | 3016 |
| Klebsiella pneumoniae subsp. | 434 | 1398 | 2209 | 3017 |
| pneumoniae 1084 | | | | |
| Klebsiella pneumoniae | 435 | 1399 | 2210 | 3018 |
| Klebsiella pneumoniae subsp. | 436 | 1400 | 2211 | 3019 |
| pneumoniae NTUH-K2044 | | | | |
| Vibrio parahaemolyticus | 437 | 1401 | 2212 | 3020 |
| Vibrio parahaemolyticus | 438 | 1402 | 2213 | 3021 |
| Arsenophonus endosymbiont of | 439 | 1403 | 2214 | 3022 |
| Apis mellifera | | | | |
| Vibrio parahaemolyticus | 440 | 1404 | 2215 | 3023 |
| Enterobacter kobei | 441 | 1405 | 2216 | 3024 |
| Enterobacter kobei | 442 | 1406 | 2217 | 3025 |
| Arsenophonus apicola | 443 | 1407 | 2218 | 3026 |
| Vibrio sp. SCSIO 43153 | 444 | 1408 | 2219 | 3027 |
| Vibrio campbellii HY01 | 445 | 1409 | 2220 | 3028 |
| Vibrio owensii | 446 | 1410 | 2221 | 3029 |
| Vibrio jasicida | 447 | 1411 | 2222 | 3030 |
| Enterobacter roggenkampii | 448 | 1412 | 2223 | 3031 |
| Shewanella sp. SM95 | 449 | 1413 | 2224 | 3032 |
| Cupriavidus taiwanensis | 450 | 1414 | 2225 | 3033 |
| Pseudomonas aeruginosa | 451 | 1415 | 2226 | 3034 |
| Pseudomonas aeruginosa | 452 | 1416 | 2227 | 3035 |
| Photobacterium leiognathi | 453 | 1417 | 2228 | 3036 |
| Zoogloea oryzae | 454 | 1418 | 2229 | 3037 |
| Janthinobacterium sp. FT14W | 455 | 1419 | 2230 | 3038 |
| Pandoraea iniqua | 456 | 1420 | 2231 | 3039 |
| Klebsiella pneumoniae | 457 | 1421 | 2232 | 3040 |
| Klebsiella pneumoniae subsp. | 458 | 1422 | 2233 | 3041 |
| pneumoniae | | | | |
| Aeromonas hydrophila | 459 | 1423 | 2234 | 3042 |
| Aeromonas hydrophila | 460 | 1424 | 2235 | 3043 |
| Aeromonas sp. QDB39 | 461 | 1425 | 2236 | 3044 |
| Aeromonas aquatica | 462 | 1426 | 2237 | 3045 |
| Pseudomonas sp. GD03919 | 463 | 1427 | 2238 | 3046 |
| Pseudomonas aeruginosa | 464 | 1428 | 2239 | 3047 |
| Pseudomonas aeruginosa | 465 | 1429 | 2240 | 3048 |
| Pseudomonas sp. GD03875 | 466 | 1430 | 2241 | 3049 |
| Stutzerimonas stutzeri A1501 | 467 | 1431 | 2242 | 3050 |
| Pseudomonas migulae | 468 | 1432 | 2243 | 3051 |
| Pseudomonas campi | 469 | 1433 | 2244 | 3052 |
| Litoribrevibacter albus | 470 | 1434 | 2245 | 3053 |
| Neisseria elongata subsp. glycolytica | 471 | 1435 | 2246 | 3054 |
| ATCC 29315 | | | | |
| Neisseria flavescens NRL30031/H210 | 472 | 1436 | 2247 | 3055 |
| Klebsiella pneumoniae subsp. | 473 | 1437 | 2248 | 3056 |
| pneumoniae | | | | |
| Janthinobacterium sp. 67 | 474 | 1438 | 2249 | 3057 |
| Duganella sacchari | 475 | 1439 | 2250 | 3058 |
| Burkholderia sp. B21-005 | 476 | 1440 | 2251 | 3059 |
| Burkholderia cepacia | 477 | 1441 | 2252 | 3060 |
| Paraglaciecola sp. T6c | 478 | 1442 | 2253 | 3061 |
| Idiomarina ramblicola | 479 | 1443 | 2254 | 3062 |
| Pseudomonas syringae pv. castaneae | 480 | 1444 | 2255 | 3063 |
| Pseudomonas syringae pv. castaneae | 481 | 1445 | 2256 | 3064 |
| Pseudomonas amygdali pv. loropetali | 482 | 1446 | 2257 | 3065 |
| Aeromonas dhakensis | 483 | 1447 | 2258 | 3066 |
| Aeromonas dhakensis | 484 | 1448 | 2259 | 3067 |
| Vibrio vulnificus | 485 | 1449 | 2260 | 3068 |
| Vibrio parahaemolyticus | 486 | 1450 | 2261 | 3069 |
| Vibrio parahaemolyticus | 487 | 1451 | 2262 | 3070 |
| Neisseria elongata subsp. glycolytica | 488 | 1452 | 2263 | 3071 |
| Neisseria elongata subsp. glycolytica | 489 | 1453 | 2264 | 3072 |
| ATCC 29315 | | | | |
| Neisseria flavescens | 490 | 1454 | 2265 | 3073 |
| Wohlfahrtiimonas chitiniclastica | 491 | 1455 | 2266 | 3074 |
| Desulfurivibrio alkaliphilus AHT 2 | 492 | 1456 | 2267 | 3075 |
| uncultured Alcanivorax sp. | 493 | 1457 | 2268 | 3076 |
| Collimonas arenae | 494 | 1458 | 2269 | 3077 |
| Yersinia ruckeri | 495 | 1459 | 2270 | 3078 |
| Yersinia pseudotuberculosis | 496 | 1460 | 2271 | 3079 |
| Yersinia pseudotuberculosis | 497 | 1461 | 2272 | 3080 |
| Yersinia aleksiciae | 498 | 1462 | 2273 | 3081 |
| Pseudomonas aeruginosa | 499 | 1463 | 2274 | 3082 |
| Pseudomonas aeruginosa | 500 | 1464 | 2275 | 3083 |
| Klebsiella sp. ZYC-1 | 501 | 1465 | 2276 | 3084 |
| Aeromonas media | 502 | 1466 | 2277 | 3085 |
| Vibrio harveyi | 503 | 1467 | 2278 | 3086 |
| Vibrio harveyi | 504 | 1468 | 2279 | 3087 |
| Alloalcanivorax xenomutans | 505 | 1469 | 2280 | 3088 |
| Nitratidesulfovibrio vulgaris DP4 | 506 | 1470 | 2281 | 3089 |
| Vibrio paracholerae HE-16 | 507 | 1471 | 2282 | 3090 |
| Vibrio cholerae | 508 | 1472 | 2283 | 3091 |
| Pseudomonas aeruginosa | 509 | 1473 | 2284 | 3092 |
| Pseudomonas sp. GD04042 | 510 | 1474 | 2285 | 3093 |
| Pseudomonas sp. BAY1663 | 511 | 1475 | 2286 | 3094 |
| Pseudomonas aeruginosa | 512 | 1476 | 2287 | 3095 |
| Pseudomonas aeruginosa | 513 | 1477 | 2288 | 3096 |
| Agarivorans gilvus | 514 | 1478 | 2289 | 3097 |
| Pseudomonas aeruginosa | 515 | 1479 | 2290 | 3098 |
| Marinomonas sp. SBI8L | 516 | 1480 | 2291 | 3099 |
| Aliivibrio fischeri | 517 | 1481 | 2292 | 3100 |
| Pseudomonas kuykendallii | 518 | 1482 | 2293 | 3101 |
| Pseudomonas aeruginosa | 519 | 1483 | 2294 | 3102 |
| Pseudomonas sp. FEMGT703P | 520 | 1484 | 2295 | 3103 |
| Vibrio sp. G41H | 521 | 1485 | 2296 | 3104 |
| Vibrio splendidus | 522 | 1486 | 2297 | 3105 |
| Stutzerimonas kunmingensis | 523 | 1487 | 2298 | 3106 |
| Vibrio splendidus | 524 | 1488 | 2299 | 3107 |
| Vibrio sp. ZF 223 | 525 | 1489 | 2300 | 3108 |
| Vibrio sp. ZF 223 | 526 | 1490 | 2301 | 3109 |
| Shewanella algae | 527 | 1491 | 2302 | 3110 |
| Vibrio crassostreae | 528 | 1492 | 2303 | 3111 |
| Pseudomonas aeruginosa | 529 | 1493 | 2304 | 3112 |
| Stutzerimonas stutzeri | 530 | 1494 | 2305 | 3113 |
| Colwellia sp. Arc7-635 | 531 | 1495 | 2306 | 3114 |
| Pseudomonas aeruginosa | 532 | 1496 | 2307 | 3115 |
| Pseudomonas aeruginosa | 533 | 1497 | 2308 | 3116 |
| Pseudomonas aeruginosa | 534 | 1498 | 2309 | 3117 |
| Pseudomonas aeruginosa | 535 | 1499 | 2310 | 3118 |
| Pseudomonas aeruginosa | 536 | 1500 | 2311 | 3119 |
| Pseudomonas aeruginosa | 537 | 1501 | 2312 | 3120 |
| Pseudomonas aeruginosa | 538 | 1502 | 2313 | 3121 |
| Pseudoalteromonas sp. 2CM28B | 539 | 1503 | ATGT | 3122 |
| Pseudoalteromonas aurantia | 540 | 1504 | 2314 | 3123 |
| Pseudoalteromonas sp. S3260 | 541 | 1505 | ATGT | 3124 |
| Pseudoalteromonas sp. S3260 | 542 | 1506 | ATGT | 3125 |
| Pseudoalteromonas sp. MT33b | 543 | 1507 | ATGT | 3126 |
| Pseudomonas vanderleydeniana | 544 | 1508 | 2315 | 3127 |
| Pseudoalteromonas sp. bablab_jr011 | 545 | 1509 | ATGT | 3128 |
| Shewanella algae | 546 | 1510 | 2316 | 3129 |
| Stutzerimonas stutzeri | 547 | 1511 | 2317 | 3130 |
| Pseudomonas aeruginosa | 548 | 1512 | 2318 | 3131 |
| Pseudomonas aeruginosa | 549 | 1513 | 2319 | 3132 |
| Pantoea ananatis | 550 | 1514 | 2320 | 3133 |
| Klebsiella pneumoniae | 551 | 1515 | 2321 | 3134 |
| Escherichia coli | 552 | 1516 | 2322 | 3135 |
| Vibrio crassostreae | 553 | 1517 | 2323 | 3136 |
| Rheinheimera oceanensis | 554 | 1518 | 2324 | 3137 |
| Klebsiella variicola | 555 | 1519 | 2325 | 3138 |
| Vibrio parahaemolyticus | 556 | 1520 | 2326 | 3139 |
| Vibrio parahaemolyticus | 557 | 1521 | 2327 | 3140 |
| Klebsiella michiganensis E718 | 558 | 1522 | 2328 | 3141 |
| Vibrio parahaemolyticus | 559 | 1523 | 2329 | 3142 |
| Vibrio parahaemolyticus | 560 | 1524 | 2330 | 3143 |
| Vibrio parahaemolyticus | 561 | 1525 | 2331 | 3144 |
| Vibrio parahaemolyticus S152 | 562 | 1526 | 2332 | 3145 |
| Enterobacter roggenkampii | 563 | 1527 | 2333 | 3146 |
| Vibrio antiquarius | 564 | 1528 | 2334 | 3147 |
| Vibrio alginolyticus | 565 | 1529 | 2335 | 3148 |
| Vibrio parahaemolyticus | 566 | 1530 | 2336 | 3149 |
| Vibrio parahaemolyticus | 567 | 1531 | 2337 | 3150 |
| Klebsiella variicola | 568 | 1532 | 2338 | 3151 |
| Vibrio fluvialis | 569 | 1533 | 2339 | 3152 |
| Vibrio fluvialis | 570 | 1534 | 2340 | 3153 |
| Vibrio cholerae | 571 | 1535 | 2341 | 3154 |
| Vibrio parahaemolyticus | 572 | 1536 | 2342 | 3155 |
| Klebsiella pneumoniae | 573 | 1537 | 2343 | 3156 |
| Klebsiella pneumoniae | 574 | 1538 | 2344 | 3157 |
| Vibrio alginolyticus | 575 | 1539 | 2345 | 3158 |
| Vibrio alginolyticus | 576 | 1540 | 2346 | 3159 |
| Vibrio alginolyticus | 577 | 1541 | 2347 | 3160 |
| Klebsiella pneumoniae subsp. | 578 | 1542 | 2348 | 3161 |
| pneumoniae | | | | |
| Klebsiella pneumoniae subsp. | 579 | 1543 | 2349 | 3162 |
| pneumoniae | | | | |
| Klebsiella pneumoniae | 580 | 1544 | 2350 | 3163 |
| Klebsiella pneumoniae | 581 | 1545 | 2351 | 3164 |
| Klebsiella pneumoniae | 582 | 1546 | 2352 | 3165 |
| Cupriavidus basilensis OR16 | 583 | 1547 | 2353 | 3166 |
| Pseudomonas syringae pv. actinidiae | 584 | 1548 | 2354 | 3167 |
| ICMP 19073 | | | | |
| Pseudomonas syringae pv. actinidiae | 585 | 1549 | 2355 | 3168 |
| ICMP 19071 | | | | |
| Photobacterium leiognathi lrivu.4.1 | 586 | 1550 | 2356 | 3169 |
| Photobacterium leiognathi lrivu.4.1 | 587 | 1551 | 2357 | 3170 |
| Pseudomonas parafulva NBRC 16636 = | 588 | 1552 | 2358 | 3171 |
| DSM 17004 | | | | |
| Pseudomonas parafulva NBRC 16636 = | 589 | 1553 | 2359 | 3172 |
| DSM 17004 | | | | |
| Aeromonas caviae | 590 | 1554 | 2360 | 3173 |
| Pseudomonas syringae pv. actinidiae | 591 | 1555 | 2361 | 3174 |
| ICMP 19073 | | | | |
| Pseudomonas syringae pv. actinidiae | 592 | 1556 | 2362 | 3175 |
| Pseudomonas syringae pv. actinidiae | 593 | 1557 | 2363 | 3176 |
| Burkholderia pseudomallei MSHR7527 | 594 | 1558 | 2364 | 3177 |
| Burkholderia pseudomallei MSHR7527 | 595 | 1559 | 2365 | 3178 |
| Aeromonas caviae | 596 | 1560 | 2366 | 3179 |
| Aeromonas caviae | 597 | 1561 | 2367 | 3180 |
| Pectobacterium brasiliense | 598 | 1562 | 2368 | 3181 |
| Photobacterium leiognathi | 599 | 1563 | 2369 | 3182 |
| Janthinobacterium sp. HH107 | 600 | 1564 | 2370 | 3183 |
| Duganella sp. CF517 | 601 | 1565 | 2371 | 3184 |
| Aeromonas hydrophila | 602 | 1566 | 2372 | 3185 |
| Aeromonas hydrophila | 603 | 1567 | 2373 | 3186 |
| Vibrio casei | 604 | 1568 | 2374 | 3187 |
| Aeromonas veronii | 605 | 1569 | 2375 | 3188 |
| Vibrio splendidus | 606 | 1570 | 2376 | 3189 |
| Pseudomonas syringae pv. cerasicola | 607 | 1571 | 2377 | 3190 |
| Pseudomonas syringae pv. cerasicola | 608 | 1572 | 2378 | 3191 |
| Pseudomonas amygdali pv. | 609 | 1573 | 2379 | 3192 |
| morsprunorum | | | | |
| Photobacterium phosphoreum | 610 | 1574 | 2380 | 3193 |
| Photobacterium phosphoreum | 611 | 1575 | 2381 | 3194 |
| Photobacterium leiognathi subsp. | 612 | 1576 | 2382 | 3195 |
| mandapamensis | | | | |
| Photobacterium leiognathi subsp. | 613 | 1577 | 2383 | 3196 |
| mandapamensis | | | | |
| Photobacterium kishitanii | 614 | 1578 | 2384 | 3197 |
| Photobacterium sp. GB-50 | 615 | 1579 | 2385 | 3198 |
| Paraburkholderia sp. BL6669N2 | 616 | 1580 | 2386 | 3199 |
| Chromatocurvus halotolerans | 617 | 1581 | 2387 | 3200 |
| Paraburkholderia sp. RAU2J | 618 | 1582 | 2388 | 3201 |
| Burkholderia stabilis | 619 | 1583 | 2389 | 3202 |
| Nitrincola iocasae | 620 | 1584 | 2390 | 3203 |
| Klebsiella michiganensis | 621 | 1585 | 2391 | 3204 |
| Aeromonas media | 622 | 1586 | 2392 | 3205 |
| Aeromonas media | 623 | 1587 | 2393 | 3206 |
| Aeromonas media | 624 | 1588 | 2394 | 3207 |
| Aeromonas hydrophila | 625 | 1589 | 2395 | 3208 |
| Aeromonas hydrophila | 626 | 1590 | 2396 | 3209 |
| Aeromonas caviae | 627 | 1591 | 2397 | 3210 |
| Aeromonas dhakensis | 628 | 1592 | 2398 | 3211 |
| Aeromonas dhakensis | 629 | 1593 | 2399 | 3212 |
| Aeromonas sp. FDAARGOS 1408 | 630 | 1594 | 2400 | 3213 |
| Aeromonas sp. FDAARGOS 1408 | 631 | 1595 | 2401 | 3214 |
| Paraburkholderia sp. F2 | 632 | 1596 | 2402 | 3215 |
| Shewanella sp. SM32 | 633 | 1597 | 2403 | 3216 |
| Pseudomonas chlororaphis | 634 | 1598 | 2404 | 3217 |
| Pseudomonas chlororaphis | 635 | 1599 | 2405 | 3218 |
| Cronobacter sakazakii NCIMB 8272 | 636 | 1600 | 2406 | 3219 |
| Vibrio cyclitrophicus | 637 | 1601 | 2407 | 3220 |
| Escherichia coli | 638 | 1602 | 2408 | 3221 |
| Klebsiella pneumoniae | 639 | 1603 | 2409 | 3222 |
| Enterobacter hormaechei | 640 | 1604 | 2410 | 3223 |
| Vibrio parahaemolyticus | 641 | 1605 | 2411 | 3224 |
| Vibrio parahaemolyticus | 642 | 1606 | 2412 | 3225 |
| Photobacterium angustum | 643 | 1607 | 2413 | 3226 |
| Klebsiella pneumoniae | 644 | 1608 | 2414 | 3227 |
| Yersinia similis | 645 | 1609 | 2415 | 3228 |
| Enterobacter kobei | 646 | 1610 | 2416 | 3229 |
| Klebsiella aerogenes | 647 | 1611 | 2417 | 3230 |
| Pantoea stewartii | 648 | 1612 | 2418 | 3231 |
| Pantoea stewartii | 649 | 1613 | 2419 | 3232 |
| Pseudomonas amygdali pv. photiniae | 650 | 1614 | 2420 | 3233 |
| Pseudomonas amygdali pv. photiniae | 651 | 1615 | 2421 | 3234 |
| Pseudomonas amygdali pv. photiniae | 652 | 1616 | 2422 | 3235 |
| Vibrio sp. MEBiC08052 | 653 | 1617 | 2423 | 3236 |
| Photobacterium aquimaris | 654 | 1618 | 2424 | 3237 |
| Vibrio parahaemolyticus | 655 | 1619 | 2425 | 3238 |
| Klebsiella pneumoniae | 656 | 1620 | 2426 | 3239 |
| Klebsiella pneumoniae | 657 | 1621 | 2427 | 3240 |
| Klebsiella quasipneumoniae | 658 | 1622 | 2428 | 3241 |
| Klebsiella quasipneumoniae | 659 | 1623 | 2429 | 3242 |
| Klebsiella quasipneumoniae | 660 | 1624 | 2430 | 3243 |
| Klebsiella quasipneumoniae | 661 | 1625 | 2431 | 3244 |
| Burkholderia ubonensis | 662 | 1626 | 2432 | 3245 |
| Klebsiella pneumoniae | 663 | 1627 | 2433 | 3246 |
| Psychromonas sp. Urea-02u-13 | 664 | 1628 | 2434 | 3247 |
| Vibrio cyclitrophicus | 665 | 1629 | 2435 | 3248 |
| Cronobacter sakazakii | 666 | 1630 | 2436 | 3249 |
| Photobacterium angustum | 667 | 1631 | 2437 | 3250 |
| Cronobacter sakazakii | 668 | 1632 | 2438 | 3251 |
| Klebsiella pneumoniae | 669 | 1633 | 2439 | 3252 |
| Vibrio genomosp. F6 | 670 | 1634 | 2440 | 3253 |
| Aeromonas veronii | 671 | 1635 | 2441 | 3254 |
| Aeromonas veronii | 672 | 1636 | 2442 | 3255 |
| Raoultella terrigena | 673 | 1637 | 2443 | 3256 |
| Raoultella terrigena | 674 | 1638 | 2444 | 3257 |
| Aeromonas veronii | 675 | 1639 | 2445 | 3258 |
| Rheinheimera tangshanensis | 676 | 1640 | 2446 | 3259 |
| Enterobacter asburiae | 677 | 1641 | 2447 | 3260 |
| Enterobacter asburiae | 678 | 1642 | 2448 | 3261 |
| Leclercia adecarboxylata | 679 | 1643 | 2449 | 3262 |
| Escherichia coli | 680 | 1644 | 2450 | 3263 |
| Escherichia coli | 681 | 1645 | 2451 | 3264 |
| Nitrogeniibacter mangrovi | 682 | 1646 | 2452 | 3265 |
| Burkholderia contaminans | 683 | 1647 | 2453 | 3266 |
| Klebsiella grimontii | 684 | 1648 | 2454 | 3267 |
| Pantoea sp. SM3640 | 685 | 1649 | 2455 | 3268 |
| Pseudoalteromonas sp. TB41 | 686 | 1650 | ATGT | 3269 |
| Arsenophonus apicola | 687 | 1651 | 2456 | 3270 |
| Arsenophonus apicola | 688 | 1652 | 2457 | 3271 |
| Vibrio cholerae | 689 | 1653 | 2458 | 3272 |
| Serratia marcescens | 690 | 1654 | 2459 | 3273 |
| Aeromonas dhakensis | 691 | 1655 | 2460 | 3274 |
| Aeromonas veronii | 692 | 1656 | 2461 | 3275 |
| Proteus sp. FME41 | 693 | 1657 | 2462 | 3276 |
| Proteus hauseri | 694 | 1658 | 2463 | 3277 |
| Pseudidiomarina donghaiensis | 695 | 1659 | 2464 | 3278 |
| Idiomarina loihiensis | 696 | 1660 | 2465 | 3279 |
| Aeromonas sp. QDB68 | 697 | 1661 | 2466 | 3280 |
| Pseudomonas otitidis | 698 | 1662 | 2467 | 3281 |
| Serratia marcescens | 699 | 1663 | 2468 | 3282 |
| Serratia marcescens | 700 | 1664 | 2469 | 3283 |
| Neisseria dumasiana | 701 | 1665 | 2470 | 3284 |
| Neisseria dumasiana | 702 | 1666 | 2471 | 3285 |
| Neisseria dumasiana | 703 | 1667 | 2472 | 3286 |
| Neisseria sp. HMSC70E02 | 704 | 1668 | 2473 | 3287 |
| Pectobacterium colocasium | 705 | 1669 | 2474 | 3288 |
| Aeromonas jandaei | 706 | 1670 | 2475 | 3289 |
| Aeromonas aquatica | 707 | 1671 | 2476 | 3290 |
| Providencia alcalifaciens R90-1475 | 708 | 1672 | 2477 | 3291 |
| Aeromonas caviae | 709 | 1673 | 2478 | 3292 |
| Ignatzschineria cameli | 710 | 1674 | 2479 | 3293 |
| Ignatzschineria cameli | 711 | 1675 | 2480 | 3294 |
| Ignatzschineria indica | 712 | 1676 | 2481 | 3295 |
| Ignatzschineria indica | 713 | 1677 | 2482 | 3296 |
| Photobacterium damselae subsp. | 714 | 1678 | 2483 | 3297 |
| damselae | | | | |
| Chromobacterium amazonense | 715 | 1679 | 2484 | 3298 |
| Photorhabdus namnaonensis | 716 | 1680 | 2485 | 3299 |
| Providencia rustigianii | 717 | 1681 | 2486 | 3300 |
| Providencia alcalifaciens Dmel2 | 718 | 1682 | 2487 | 3301 |
| Providencia alcalifaciens Dmel2 | 719 | 1683 | 2488 | 3302 |
| Plesiomonas shigelloides | 720 | 1684 | 2489 | 3303 |
| Aeromonas hydrophila | 721 | 1685 | 2490 | 3304 |
| Aeromonas hydrophila | 722 | 1686 | 2491 | 3305 |
| Morganella morganii | 723 | 1687 | 2492 | 3306 |
| Aeromonas caviae | 724 | 1688 | 2493 | 3307 |
| Aeromonas dhakensis | 725 | 1689 | 2494 | 3308 |
| Aeromonas dhakensis | 726 | 1690 | 2495 | 3309 |
| Aeromonas veronii | 727 | 1691 | 2496 | 3310 |
| Neisseria zalophi | 728 | 1692 | 2497 | 3311 |
| Aeromonas hydrophila | 729 | 1693 | 2498 | 3312 |
| Aureimonas sp. Leaf324 | 730 | 1694 | 2499 | 3313 |
| Albimonas donghaensis | 731 | 1695 | 2500 | 3314 |
| Inquilinus limosus | 732 | 1696 | 2501 | 3315 |
| Brevundimonas sp. | 733 | 1697 | 2502 | 3316 |
| Roseobacter sp. TSBP12 | 734 | 1698 | 2503 | 3317 |
| Pseudoxanthomonas composti | 735 | 1699 | 2504 | 3318 |
| Priestia taiwanensis | 736 | 1700 | 2505 | 3319 |
| Bacillus thuringiensis | 737 | 1701 | 2506 | 3320 |
| Rhizobium sp. BR 318 | 738 | 1702 | 2507 | 3321 |
| Stenotrophomonas maltophilia | 739 | 1703 | 2508 | 3322 |
| Devosia sp. 63-57 | 740 | 1704 | 2509 | 3323 |
| Rhizobium sp. CCGE 510 | 741 | 1705 | 2510 | 3324 |
| Bacillus pacificus | 742 | 1706 | 2511 | 3325 |
| Bacillus cereus group sp. BcHK28 | 743 | 1707 | 2512 | 3326 |
| Lysinibacillus fusiformis | 744 | 1708 | 2513 | 3327 |
| Brucella tritici | 745 | 1709 | 2514 | 3328 |
| Rhizobium lentis | 746 | 1710 | 2515 | 3329 |
| Brucella intermedia | 747 | 1711 | 2516 | 3330 |
| Rhodobacter capsulatus Y262 | 748 | 1712 | 2517 | 3331 |
| Agrobacterium tumefaciens | 749 | 1713 | 2518 | 3332 |
| Agrobacterium tumefaciens | 750 | 1714 | 2519 | 3333 |
| Sulfitobacter sabulilitoris | 751 | 1715 | 2520 | 3334 |
| Brucella endophytica | 752 | 1716 | 2521 | 3335 |
| Rhizobium glycinendophyticum | 753 | 1717 | 2522 | 3336 |
| Rhizobium ruizarguesonis | 754 | 1718 | 2523 | 3337 |
| Flavonifractor sp. An100 | 755 | 1719 | 2524 | 3338 |
| Hansschlegelia plantiphila | 756 | 1720 | 2525 | 3339 |
| Mitsuokella multacida | 757 | 1721 | 2526 | 3340 |
| Lysinibacillus fusiformis ZB2 | 758 | 1722 | 2527 | 3341 |
| Bacillus inaquosorum | 759 | 1723 | 2528 | 3342 |
| Lysinibacillus sp. LK3 | 760 | 1724 | 2529 | 3343 |
| Clostridiales bacterium KLE1615 | 761 | 1725 | 2530 | 3344 |
| Devosia elaeis | 762 | 1726 | 2531 | 3345 |
| Lysinibacillus sp. AR18-8 | 763 | 1727 | 2532 | 3346 |
| Paracoccus sediminis | 764 | 1728 | 2533 | 3347 |
| Paracoccus sediminis | 765 | 1729 | 2534 | 3348 |
| Minwuia thermotolerans | 766 | 1730 | 2535 | 3349 |
| Blautia sp. TF12-12AT | 767 | 1731 | 2536 | 3350 |
| Cytobacillus solani | 768 | 1732 | 2537 | 3351 |
| Bradyrhizobium daqingense | 769 | 1733 | 2538 | 3352 |
| Ruegeria sp. A3M17 | 770 | 1734 | 2539 | 3353 |
| Paenalkalicoccus suaedae | 771 | 1735 | 2540 | 3354 |
| Polymorphobacter sp. PAMC 29334 | 772 | 1736 | 2541 | 3355 |
| Bacillus atrophaeus | 773 | 1737 | 2542 | 3356 |
| Acinetobacter baumannii | 774 | 1738 | 2543 | 3357 |
| Acinetobacter baumannii | 775 | 1739 | 2544 | 3358 |
| Acinetobacter baumannii | 776 | 1740 | 2545 | 3359 |
| Allomuricauda eckloniae | 777 | 1741 | 2546 | 3360 |
| Acinetobacter pittii | 778 | 1742 | 2547 | 3361 |
| Altererythrobacter sp. FM1 | 779 | 1743 | 2548 | 3362 |
| Acinetobacter haemolyticus | 780 | 1744 | 2549 | 3363 |
| Altericroceibacterium spongiae | 781 | 1745 | 2550 | 3364 |
| Azospirillum brasilense | 782 | 1746 | 2551 | 3365 |
| Oceanobacillus sp. J11TS1 | 783 | 1747 | 2552 | 3366 |
| Rhizobium anhuiense bv. trifolii | 784 | 1748 | 2553 | 3367 |
| Bacillus inaquosorum | 785 | 1749 | 2554 | 3368 |
| Bacillus inaquosorum | 786 | 1750 | 2555 | 3369 |
| Alkalihalobacillus pseudalcaliphilus | 787 | 1751 | 2556 | 3370 |
| Gluconobacter morbifer G707 | 788 | 1752 | 2557 | 3371 |
| Lactobacillus intestinalis | 789 | 1753 | 2558 | 3372 |
| Pseudovibrio sp. Tun.PSC04-5.I4 | 790 | 1754 | 2559 | 3373 |
| Bradyrhizobium sp. NFR13 | 791 | 1755 | 2560 | 3374 |
| Lysinibacillus sphaericus | 792 | 1756 | 2561 | 3375 |
| Sphingomonas sp. UV9 | 793 | 1757 | 2562 | 3376 |
| Rhizobium ruizarguesonis | 794 | 1758 | 2563 | 3377 |
| Rhizobium ruizarguesonis | 795 | 1759 | 2564 | 3378 |
| Mesorhizobium composti | 796 | 1760 | 2565 | 3379 |
| Rhizobium laguerreae | 797 | 1761 | 2566 | 3380 |
| Sinorhizobium saheli | 798 | 1762 | 2567 | 3381 |
| Sinorhizobium saheli | 799 | 1763 | 2568 | 3382 |
| Albimonas pacifica | 800 | 1764 | 2569 | 3383 |
| Novacetimonas cocois | 801 | 1765 | 2570 | 3384 |
| Bacillus cereus | 802 | 1766 | 2571 | 3385 |
| Bacillus cereus biovar anthracis str. CI | 803 | 1767 | 2572 | 3386 |
| Bacillus cereus | 804 | 1768 | 2573 | 3387 |
| Paracoccus sediminicola | 805 | 1769 | 2574 | 3388 |
| Paracoccus alkenifer | 806 | 1770 | 2575 | 3389 |
| Paracoccus pantotrophus | 807 | 1771 | 2576 | 3390 |
| Exiguobacterium marinum DSM 16307 | 808 | 1772 | 2577 | 3391 |
| Rhizobium leguminosarum | 809 | 1773 | 2578 | 3392 |
| Rhizobium leguminosarum | 810 | 1774 | 2579 | 3393 |
| Salinicoccus cyprini | 811 | 1775 | 2580 | 3394 |
| Psychrobacillus soli | 812 | 1776 | 2581 | 3395 |
| Agrobacterium tumefaciens LBA4213 (Ach5) | 813 | 1777 | 2582 | 3396 |
| Agrobacterium deltaense RV3 | 814 | 1778 | 2583 | 3397 |
| Lysinibacillus sp. A1 | 815 | 1779 | 2584 | 3398 |
| Lysinibacillus fusiformis | 816 | 1780 | 2585 | 3399 |
| Lysinibacillus sphaericus | 817 | 1781 | 2586 | 3400 |
| Larkinella punicea | 818 | 1782 | 2587 | 3401 |
| Rhodobacter capsulatus DE442 | 819 | 1783 | 2588 | 3402 |
| Rhodobacter capsulatus SB 1003 | 820 | 1784 | 2589 | 3403 |
| Acinetobacter nosocomialis | 821 | 1785 | 2590 | 3404 |
| Acinetobacter baumannii | 822 | 1786 | 2591 | 3405 |
| Acinetobacter baumannii | 823 | 1787 | 2592 | 3406 |
| Bacillus subtilis | 824 | 1788 | 2593 | 3407 |

It is understood that the foregoing detailed description and accompanying examples are merely illustrative and are not to be taken as limitations upon the scope of the disclosure, which is defined solely by the appended claims and their equivalents.

All publications and patents mentioned in the above specification are herein incorporated by reference as if expressly set forth herein. Various changes and modifications to the disclosed embodiments will be apparent to those skilled in the art and may be made without departing from the spirit and scope thereof.

1. A system for guided DNA synthesis comprising:
a defense-associated reverse transcriptase (DRT), or a nucleic acid encoding thereof, and
one or more engineered target nucleic acids each configured for recognition by the DRT and comprising one or more sequences of interest, or a nucleic acid encoding thereof.

2. The system of claim 1, wherein the DRT comprises an amino acid sequence having at least 70% identity with any of SEQ ID NOs: 1-824.

3. The system of claim 1, wherein the DRT comprises an amino acid sequence of any of SEQ ID NOs: 1-824.

4. The system of claim 1, wherein each of the one or more engineered target nucleic acids comprise:
a template region comprising the one or more sequences of interest;
a 3′ region configured to recruit the DRT to the engineered target nucleic acid; and
a 5′ region.

5. The system of claim 4, wherein
the template region abuts a short basal stem;
the 5′ end of the template sequence is homologous to the sequence adjacent to the 3′ of the template sequence in the short basal stem;
the 3′ region comprises one or more stem-loops; and/or
the 5′ region comprises a single stem-loop.

6. The system of claim 1, wherein the system further comprises one or more genomic engineering reagents selected from: single-stranded annealing proteins (SSAP), guide RNAs, DNA endonucleases, ribonucleases, transcriptional activators, transcriptional repressors, histone-modifying proteins, integrases, recombinases, DNA polymerases, and combinations thereof.

7. A method for guided DNA synthesis comprising:
contacting a defense-associated reverse transcriptase (DRT) with one or more engineered target nucleic acids configured for recognition by the DRT, each comprising one or more sequences of interest.

8. The method of claim 7, wherein the method produces a concatemeric DNA product.

9. The method of claim 7, wherein each of the one or more engineered target nucleic acids comprise:
a template region comprising the one or more sequences of interest;
a 3′ region configured to recruit the DRT to the engineered target nucleic acid; and
a 5′ region.

10. The method of claim 9, wherein
the template region abuts a short basal stem;
the 5′ end of the template sequence is homologous to the sequence adjacent to the 3′ of the template sequence in the short basal stem;
the 3′ region comprises one or more stem-loops; and/or
the 5′ region comprises a single stem-loop

11. The method of claim 9, wherein the DNA product comprises at least one sequence complementary to the one or more sequences of interest.

12. The method of claim 7, wherein the DRT comprises an amino acid sequence having at least 70% identity with any of SEQ ID NOs: 1-824.

13. The method of claim 7, wherein the DRT comprises an amino acid sequence of any of SEQ ID NOs: 1-824.

14. The method of claim 7, wherein the contacting is in a cell and the method comprises introducing into the cell the DRT and the engineered nucleic acid, or one or more nucleic acids encoding thereof.

15. The method of claim 14, wherein the cell is in vivo or in vitro.

16. The system of claim 1, wherein the DRT is from or derived from a DRT2-family phage defense system.

17. The method of claim 7, wherein the DRT is from or derived from a DRT2-family phage defense system.
